(19) United States
(12) Patent Application Publication
(10) Pub. No.: US 2024/0174625 A1
(43) Pub. Date: May 30, 2024

(54) COMPOSITIONS AND METHODS FOR TREATING TAUOPATHIES

(71) Applicant: The Regents of the University of California, Oakland, CA (US)

(72) Inventors: Daniel H. Geschwind, Los Angeles, CA (US); Michael E. Jung, Los Angeles, CA (US); Liting Deng, Los Angeles, CA (US); Flora Hinz, Los Angeles, CA (US); Jennifer M. Murphy, Los Angeles, CA (US); Gaoyuan Ma, Los Angeles, CA (US); Robert Damoiseaux, Los Angeles, CA (US)

(21) Appl. No.: 18/277,522

(22) PCT Filed: Feb. 18, 2022
(86) PCT No.: PCT/US22/17032

§ 371 Date: Aug. 16, 2023

Related U.S. Application Data
(60) Provisional application No. 63/151,453, filed on Feb. 19, 2021.

(51) Int. Cl. C07D 275/04; A61P 25/28; C07D 213/74; C07D 217/22; C07D 239/42; C07D 239/94; C07D 261/20; C07D 417/04; C07D 417/12; C07D 513/04

(52) U.S. Cl. CPC C07D 275/04; A61P 25/28; C07D 213/74; C07D 217/22; C07D 239/42; C07D 239/94; C07D 261/20; C07D 417/04; C07D 417/12; C07D 513/04

(57) ABSTRACT

Disclosed are compounds and methods for treating tauopathy.

### RELATED APPLICATIONS

This application claims the benefit of U.S. Provisional Application No. 63/141,453, filed Feb. 19, 2021, the contents of which are fully incorporated by reference herein.

### BACKGROUND

Protein abnormalities, misfolding, or pathological aggregates are key features of various diseases or disorders, collectively named as proteopathies, such as tau-associated neurodegenerative diseases (collectively named tauopathies, a specific form of proteopathy involving the microtubule associated protein, tau; gene name MAPT), Alzheimer's Disease and other dementias, Parkinson's Disease, Huntington's Disease, Amyotrophic Lateral Sclerosis, and Spinocerebral Ataxia, all of which are major unmet public health need facing humanity.

Puromycin-sensitive aminopeptidase (also known as PSA, AAP-S, MP100; gene name NPEPPS) was originally identified through an unbiased, cross-species genome-wide screen for endogenous modulators that attenuate tau-induced neurodegeneration (Karsten et al., Neuron 2006). It was demonstrated that loss of NPEPPS promotes neurodegeneration, whereas upregulation of NPEPPS safely reduces tau levels and mitigates neurodegenerative phenotypes in animal models of tauopathies. Analyses of human brain from dementia patients show that NPEPPS protein levels are reduced in prefrontal cortex (a brain region that is more vulnerable to neurodegeneration) as compared to cerebellum (a brain region that is more resistant to neurodegeneration), indicating NPEPPS is neuroprotective (Karsten et al., Neuron 2006). Therefore, targeting reduction of neuropathological proteins via activating or enhancing the activity of NPEPPS is a promising approach to tauopathy.

### SUMMARY OF THE INVENTION

The present disclosure provides compounds of formula I:

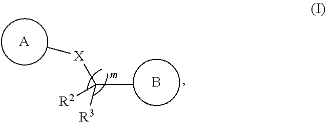

and pharmaceutically acceptable salts thereof, wherein:
X is C(R⁰)₂, NR¹, O or S;
ring A is C₆₋₁₀ aryl or 5- to 10-membered heteroaryl;
ring B is C₆₋₁₄ aryl or 5- to 10-membered heteroaryl;
R⁰ is halogen, amino, hydroxyl, alkoxy, cyano, nitro, alkyl, alkenyl, alkynyl, carbocyclyl, heterocyclyl, aryl, or heteroaryl;
R¹ is hydrogen, sulfonyl, alkyl, aralkyl, alkenyl, alkynyl, carbocyclyl, heterocyclyl, aryl, or heteroaryl;
R² and R³ independently are hydrogen, alkyl, alkenyl, alkynyl, carbocyclyl, heterocyclyl, aryl, or heteroaryl; or
R², R³, and the carbon atom to which they are connected, complete an oxo group (C═O); or
R³, ring B, and the intervening atoms, complete a carbocyclyl, heterocyclyl, aryl, or heteroaryl;
m is 0, 1, or 2, preferably 1, and
provided the compound is not

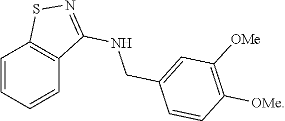 [SMILES: COc1ccc(CNc2nsc3ccccc23)cc1OC]

In certain aspects, the present disclosure provides methods of treating a proteopathy, comprising administering to a subject in need thereof a compound disclosed herein.

### BRIEF DESCRIPTION OF THE DRAWINGS

FIGS. 1A-E. Puromycin-sensitive aminopeptidase (NPEPPS; PSA) is an inhibitor of tau-induced neuro-degeneration (Karsten et al., Neuron 2006). (FIG. 1A) Altered levels of NPEPPS in Tau^{P301L} mouse brain is identified by a large-scale microarray gene expression experiment and validated by northern blot. n=6±SD. (FIG. 1B) The gl-Tau^{P301L} (left) phenotype was attenuated when co-expressed with gl-dNPEPPS (right) in drosophila. Scale bars, 100 mm. (FIG. 1C) NPEPPS degraded human recombinant tau protein in a cell-free system. (FIG. 1D) Immunoblots showed reduced levels of both Tau^{WT} and Tau^{P301L} in drosophila co-expressing dNPEPPS. (FIG. 1E) Immunoblots demonstrated NPEPPS levels are elevated in cerebellum as compared to frontal cortex in dementia patient brain (P<0.01). Each bar represents the n=6±SD.

FIGS. 2A-F. Development of a cell-based NPEPPS-specific activity assay. (FIG. 2A) Schematic of NPEPPS activity assay. (FIG. 2B) Overexpression of hNPEPPS increases fluorescence. n=12±SEM. (FIG. 2C) When NPEPPS is knocked-down using two different shRNA constructs with slightly different efficiencies, fluorescence changes as measured by the Q-AMC assay directly correlate to changes in NPEPPS protein level. n=32±SEM. (FIG. 2D) Inhibition of NPEPPS activity either by the specific NPEPPS inhibitor puromycin or the broader aminopeptidase inhibitor bestatin results in decreases in fluorescence change. n=12±SEM. (FIG. 2E) Numbers of hit compounds by chemical library in the primary screen using Q-AMC-based NPEPPS activity assay. Compounds that produced an increase in fluorescence change as a readout of NPEPPS activity over three standard deviations above the average fluorescence change for the plate (z-score>3) were classified as ‘hits’. (FIG. 2F) Summary of the screen flow to identify three series of NPEPPS enhancers/activators of distinct chemical clusters.

FIGS. 3A, B. JMM001 (Cpd.1) direct engages NPEPPS as its target. (FIG. 3A) Cell-free protein stability assay and quantification of melting temperature of purified human recombinant NPEPPS in the presence of JMM001, DMSO control, or specific NPEPPS inhibitor Puromycin as positive control. (FIG. 3B) NPEPPS melt and shift curve in HEK293T cell lysates treated with JMM001 (Cpd.1) or DMSO control in the cellular thermal shift assay. n=3±SEM.

FIG. 4. Effects of JMM compounds on NPEPPS activity (red) and cell viability (blue). JMM compounds JMM001 to JMM139 are tested in the cellular, fluorescence-based NPEPPS activity assay and assessed on toxicity profiles via the cell viability assay. Red line and left y-axis show NPEPPS activity normalized to DMSO control; Blue line and right y-axis show cell viability; dotted line marks 50% increase in NPEPPS activity (150% NPEPPS activity normalized to DMSO control). n=2-4±SEM.

FIG. 5. Effects of JMM compounds on tau reduction in mouse primary cortical neurons. n=2-4±SEM. Active JMM compounds decrease tau levels (bottom, representative immunoblotting images) and show positive correlation between NPEPPS activity assay and tau degradation (top).

FIGS. 6A-L. Preclinical potency of JMM compounds on tau and phosphorylated tau levels in dementia patient iPSC-derived neurons. Dementia patient iPSC-derived neurons, a human “disease in a dish” model of tauopathy, display tau pathological phenotypes⁵. (FIGS. 6A,E,F,H,I,K) Sample immunoblots and (FIGS. 6B,G,J,L) quantification of concentration-response curves of total tau and phospho-tau (P-Tau) levels in iPSC-derived neurons from FTD patients carrying the tau^{P301L} mutation; neurons are treated with JMM compounds for 24 hrs. (FIG. 6C) Sample immunoblots and (FIG. 6D) quantification of total tau levels in iPSC-derived neurons from age-matched healthy individuals; neurons are treated with JMM001 for 24 hrs. Total tau antibody Tau-5, phospho-Tau antibodies S396 or AT8 are used, and results are normalized to beta-actin levels. Phospho-Tau S396 antibody is used to visualize oligomers and monomers of phospho-tau: the higher molecular weight bands and 50˜75 kD molecular weight bands correspond to oligomers and monomers, respectively. AT8 (Ser202, Thr205) antibody identify aggregation-prone, disease-associated forms of phospho-tau. n=1-3±standard deviation.

FIGS. 7A-D. Preclinical potency of JMM compounds on rescuing neuronal survival in dementia patient iPSC-derived neurons. (FIG. 7A) JMM001 at 1 μM, (FIG. 7B) JMM013 at 5 μM, (FIG. 7C) JMM052 at 1 μM, (FIG. 7D) JMM067 at 5 μM improves neuronal survival in iPSC-derived neurons from FTD patients carrying the tau^{V337M} mutation compared to (FIG. 7A) DMSO or (FIGS. 7B-D) an inactive analogue JMM033 at 5 μM. Kaplan-Meier plots of (FIG. 7A) n=125 neurons or (FIGS. 7B-D) n=100 neurons pooled from 3 independent experiments.

FIGS. 8A-D. In vivo plasma pharmacokinetics profiles of JMM compounds. JMM SAR analogues (e.g., JMM013, JMM015, JMM052, JMM067, and JMM083) at 20 mg/kg i.p. display improved (FIG. 8A) plasma PK parameters, (FIG. 8B) brain levels, and (FIG. 8D) half life compared to JMM001 (20 mg/kg i.p.). Plasmas were collected at 0.5, 1, 2, 4, 8, and 24 hrs post-injection. (FIG. 8B) Brain samples were collected at time point of Cmax. (FIG. 8C) JMM001 shows high oral bioavailability. JMM001 (20 mg/kg i.v. or p.o.) was acutely administered to mice. Samples were analyzed by LC/MS. n=3-5±SEM.

FIGS. 9A-H. In vivo effects of JMM compounds on tau histopathology in a mouse model of tauopathy. Square boxes on the sagittal mouse brain atlas show the position of the immunofluorescence image. (FIGS. 9A,C,E,G) Representative immunofluorescence images from the median sample of each group and (FIGS. 9B,D,F,H) quantification of phosphorylated tau (green) in cortex of hTau^{P301S} homozygous mice or WT littermates treated with either vehicle or JMM SAR analogues (A,B) JMM013, (C,D) JMM052, (E,F) JMM067, or (G,H) JMM083 (20 mg/kg/day s.c. for 28 days starting from 4.2 months onset of tau histopathology and brains are collected at treatment day 28). Pooled gender. *P<0.05, WT-Control vs. Homo-Control; +P<0.05, Treatment vs. Vehicle, One-way ANOVA followed by Bonferroni post hoc tests or one-tailed t-tests. (FIGS. 9A,B) F_{3,21}=5.343, P<0.01; homo-JMM013 vs homo-vehicle (P<0.05, Bonferroni post hoc test). (FIGS. 9C,D) F_{2,21}=3.357, P=0.054, ns; homo-JMM052 vs homo-vehicle (P=0.071, ns, one-tailed t-test). (FIGS. 9E,F) F_{3,22}=3.530, P<0.05; homo-JMM067 vs homo-vehicle (P<0.05, one-tailed t-test). (FIGS. 9G,H) F_{3, 21}=3.576, P<0.05; homo-JMM083 vs. homo-vehicle (P<0.05, one-tailed T=t-test). Scale bar=50 μm. n=2-4 (WT) and n=6-12 (homo)±SEM.

FIGS. 10A,B. In vivo effects of JMM001 on mitigating neurodegeneration in a zebrafish model of tauopathy. JMM001 at 0.01 and 0.03 μM rescue the photoreceptor degeneration in the rhodopsin::EGFP-Tau^{P301L} zebrafish compared to DMSO control. (FIG. 10A) Sample image and (FIG. 10B) quantification. n=20-25±SEM. *P<0.05.

FIG. 11. Potential applications of NPEPPS enhancer JMM series on other neurodegenerative diseases. JMM001 (Cp 1) at 50 μM reduces protein levels of huntingtin (htt), SOD1, ataxin, and alpha-synuclein in human primary cortical neurons. Neurons were treated with 50 μM JMM001 or 10 uM bestatin for 48 hrs. N=3-4±SEM.

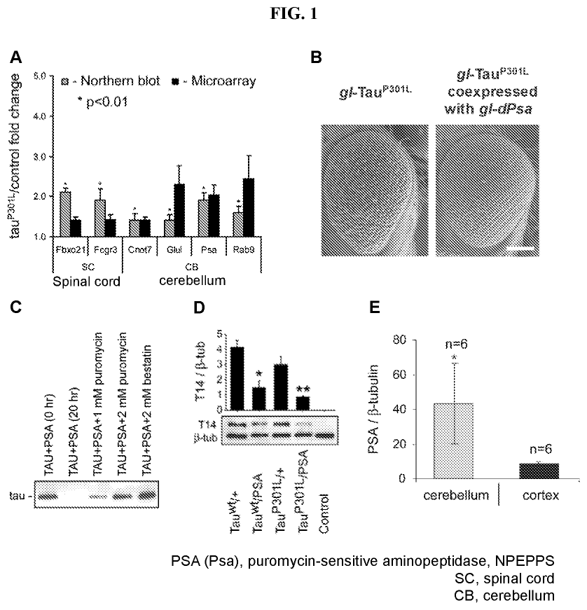

### DETAILED DESCRIPTION OF THE INVENTION

The present disclosure provides compound of formula I:

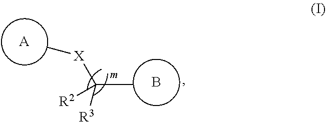

or a pharmaceutically acceptable salt thereof, wherein:
X is C(R⁰)₂, NR¹, O or S;
ring A is C₆₋₁₀ aryl or 5- to 10-membered heteroaryl;
ring B is C₆₋₁₄ aryl or 5- to 10-membered heteroaryl;
R⁰ is halogen, amino, hydroxyl, alkoxy, cyano, nitro, alkyl, alkenyl, alkynyl, carbocyclyl, heterocyclyl, aryl, or heteroaryl;
R¹ is hydrogen, sulfonyl, alkyl, aralkyl, alkenyl, alkynyl, carbocyclyl, heterocyclyl, aryl, or heteroaryl;
R² and R³ independently are hydrogen, alkyl, alkenyl, alkynyl, carbocyclyl, heterocyclyl, aryl, or heteroaryl; or
R², R³, and the carbon atom to which they are connected, complete an oxo group (C═O); or
R³, ring B, and the intervening atoms complete a carbocyclyl, heterocyclyl, aryl, or heteroaryl;
m is 0, 1, or 2, preferably 1, and
provided the compound is not

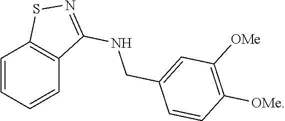 [SMILES: COc1ccc(CNc2nsc3ccccc23)cc1OC]

In certain embodiments, the compound is of formula I:

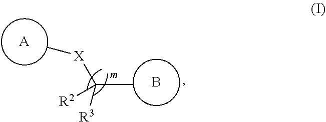

and pharmaceutically acceptable salts thereof, wherein:
X is C(R⁰)₂, NR¹, O or S;
ring A is C₆₋₁₀ aryl or 6- to 10-membered heteroaryl;
ring B is C₆₋₁₄ aryl or 6- to 10-membered heteroaryl;
R⁰ is halogen, amino, hydroxyl, alkoxy, cyano, nitro, alkyl, alkenyl, alkynyl, carbocyclyl, heterocyclyl, aryl, or heteroaryl;
R¹ is hydrogen, sulfonyl, alkyl, aralkyl, alkenyl, alkynyl, carbocyclyl, heterocyclyl, aryl, or heteroaryl;
R² and R³ independently are hydrogen, alkyl, alkenyl, alkynyl, carbocyclyl, heterocyclyl, aryl, or heteroaryl; or
R², R³, and the carbon atom to which they are connected, complete an oxo group (C═O); or
R³, ring B, and the intervening atoms, complete a carbocyclyl, heterocyclyl, aryl, or heteroaryl;
m is 0, 1, or 2, preferably 1, and
provided the compound is not

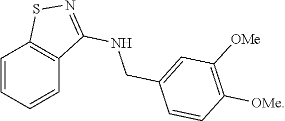 [SMILES: COc1ccc(CNc2nsc3ccccc23)cc1OC]

In certain embodiments, X is NR¹. In certain such embodiments, R¹ is hydrogen, sulfonyl (e.g., methylsulfonyl), alkyl (e.g., C₁-C₆ alkyl such as methyl or ethyl), aralkyl (e.g., optionally substituted benzyl, such as 4-trifluorobenzyl), alkenyl, alkynyl, carbocyclyl, heterocyclyl, aryl, or heteroaryl. In further embodiments, R¹ is hydrogen, sulfonyl, alkyl, aralkyl, carbocyclyl, heterocyclyl, aryl, or heteroaryl. In yet further embodiments, R¹ is hydrogen, sulfonyl (e.g., methylsulfonyl), alkyl (e.g., C₁-C₆ alkyl such as methyl or ethyl), or aralkyl (e.g., optionally substituted benzyl such as 4-trifluorobenzyl). In certain preferred embodiments, R¹ is H or methyl.

In certain embodiments, ring A is 5- to 10-membered heteroaryl (e.g., a 6- to 10-membered heteroaryl), such as a 6-membered heteroaryl and may be optionally substituted. For example, ring A can be

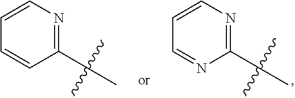 [SMILES: *c1ccccn1 ; *c1ncccn1]

each of which may be optionally substituted.

In certain embodiments, ring A is 9- to 10-membered heteroaryl which can be optionally substituted. For example ring A can be

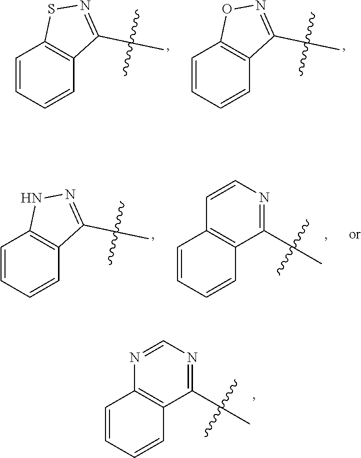 [SMILES: *c1nsc2ccccc12 ; *c1noc2ccccc12 ; *c1n[nH]c2ccccc12 ; *c1nccc2ccccc12 ; *c1ncnc2ccccc12]

preferably

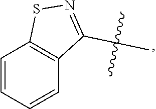 [SMILES: *c1nsc2ccccc12]

any of which can be optionally substituted.

In certain embodiments, ring A is optionally substituted

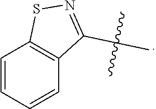 [SMILES: *c1nsc2ccccc12]

Optional substituents include one or more of bromo, chloro, fluoro, ethynyl, cyano, benzyloxy (e.g., trifluoromethyl substituted benzyloxy), trifluoromethyl, and methoxy. In certain embodiments, ring A is preferably unsubstituted or substituted with benzyloxy.

In certain embodiments, ring B is C₆₋₁₄ aryl, which can be optionally substituted. For example, ring B can be

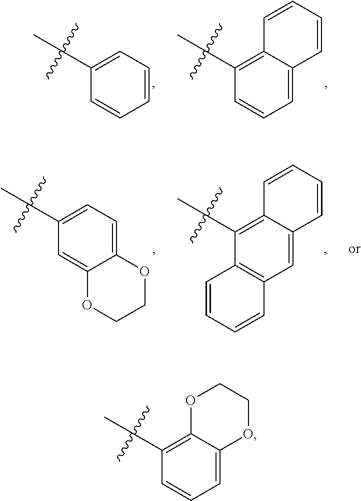 [SMILES: *c1ccccc1 ; *c1cccc2ccccc12 ; *c1ccc2c(c1)OCCO2 ; *c1c2ccccc2cc2ccccc12 ; *c1cccc2c1OCCO2]

preferably

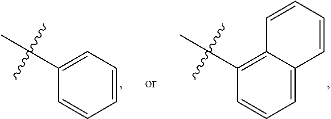 [SMILES: *c1ccccc1 ; *c1cccc2ccccc12]

each of which may be optionally substituted. In certain preferred embodiments, ring B is optionally substituted

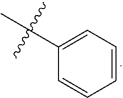 [SMILES: *c1ccccc1]

Optional substituents for ring B include one or more of benzyl, methyl, —CH₂—O-phenyl, trifluoromethyl, fluoro, chloro, bromo, trifluoromethoxy, —CO₂Me, cyano, nitro, difluoromethyl, —SCF₃, —OR⁶, —NHR⁶, or —N(R⁶)₂, wherein each R⁶ is independently hydrogen, alkyl, aryl, or heteroaryl. In certain preferred embodiments, ring B is unsubstituted or when the optional substituent is present, the optional substituent is preferably trifluoromethyl or benzyloxy.

In certain embodiments, ring B is 5- to 10-membered heteroaryl (e.g., 6- to 10-membered heteroaryl) and can be optionally substituted. For example, ring B can be

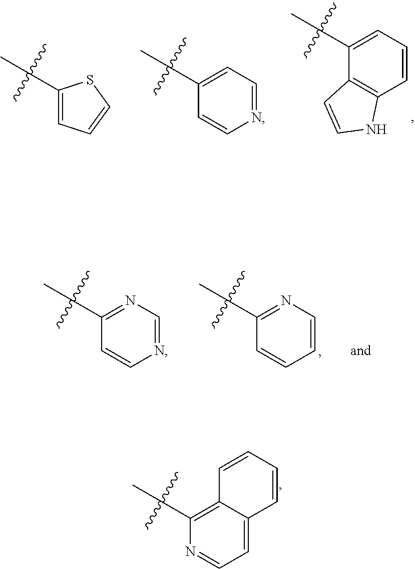 [SMILES: *c1cccs1 ; *c1ccncc1 ; *c1cccc2[nH]ccc12 ; *c1ccncn1 ; *c1ccccn1 ; *c1nccc2ccccc12]

each of which can be optionally substituted. Optional substituents include one or more of benzyl, methyl, —CH₂—O-phenyl, trifluoromethyl, fluoro, chloro, bromo, trifluoromethoxy, —CO₂Me, cyano, nitro, difluoromethyl, —SCF₃, —OR⁶, —NHR⁶, or —N(R⁶)₂, wherein each R⁶ is independently hydrogen, alkyl, aryl, or heteroaryl. In certain embodiments, ring B is unsubstituted.

In certain embodiments, the compound of formula I is a compound of formula I-1

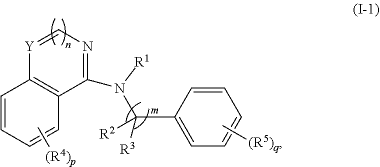

wherein:
Y is O, N, or S;
n is 0 or 1;
R⁴ and R⁵, independently for each occurrence, are halogen, cyano, nitro, alkyl, alkenyl, alkynyl, carbocyclyl, heterocyclyl, aryl, heteroaryl, —OR⁶, —NHR⁶, or —N(R⁶)₂, wherein each R⁶ is independently hydrogen, alkyl, aryl, or heteroaryl; and
   p and q independently are an integer selected from 0 to 5, as valency permits.

In certain embodiments, m is 0 or 1. For example, m can be 1.

In certain embodiments, the compound of formula I is a compound of formula I-1-a:

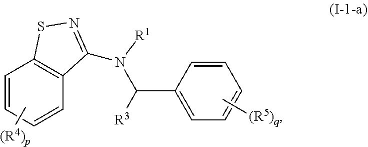

wherein:
R⁴ and R⁵, independently for each occurrence, are halogen, cyano, nitro, alkyl, alkenyl, alkynyl, carbocyclyl, heterocyclyl, aryl, or heteroaryl, —OR⁶, —NHR⁶, or —N(R⁶)₂, wherein each R⁶ is independently hydrogen, alkyl, aryl, or heteroaryl; and
   p and q independently are an integer selected from 0 to 5, as valency permits.

In certain embodiments R² is hydrogen, alkyl, aryl or heteroaryl. For example, R² can be hydrogen. In certain particular embodiments, m is 1 and R² is hydrogen.

In certain embodiments, R³ is hydrogen, alkyl (e.g., methyl), aryl (e.g., phenyl) or heteroaryl. In further embodiments, R³ is phenyl.

In certain embodiments, R², R³, and the carbon atom to which they are connected, are taken together to complete an oxo group (C═O).

In certain embodiments, R³, ring B, and the intervening atoms complete a carbocyclyl, heterocyclyl, aryl, or heteroaryl.

In certain embodiments, each R⁶ is independently aryl or alkyl optionally substituted by aryl, heteroaryl, or cycloalkyl. For example, R⁶ can be methyl,

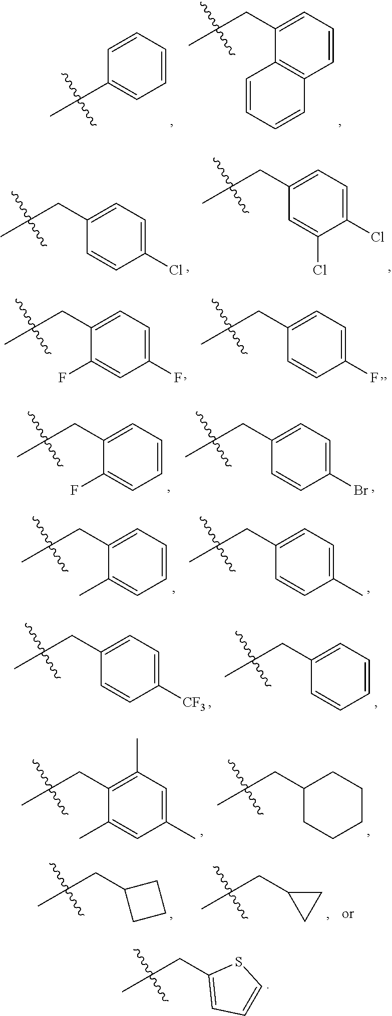

In certain embodiments, the compound of formula I is a compound of formula I-1-b:

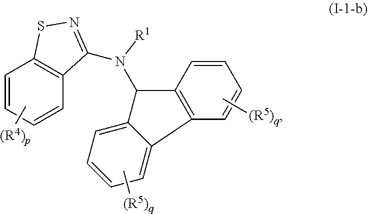

wherein:
Y is O, N, or S;
n is 0 or 1;
R⁴ and R⁵, independently for each occurrence, are halogen, amino, hydroxyl, alkoxy, cyano, nitro, alkyl, alkenyl, alkynyl, carbocyclyl, heterocyclyl, aryl, or heteroaryl; and
   p and q, independently for each occurrence, are an integer selected from 0 to 5, as valency permits.

In certain embodiments, each R⁴ is hydrogen, bromo, fluoro, ethynyl, cyano, benzyloxy, or methoxy. The benzyloxy can be optionally substituted with, for example, trifluoromethyl.

In certain embodiments, each R⁵ is hydrogen, methyl, trifluoromethyl, fluoro, chloro, bromo, methoxy, trifluoromethoxy, benzyloxy, dimethylamino, —CO₂Me, cyano, nitro, difluoromethyl, or —SCF₃.

In certain preferred embodiments, each of p and q is 0.

In certain embodiments, the compound is selected from

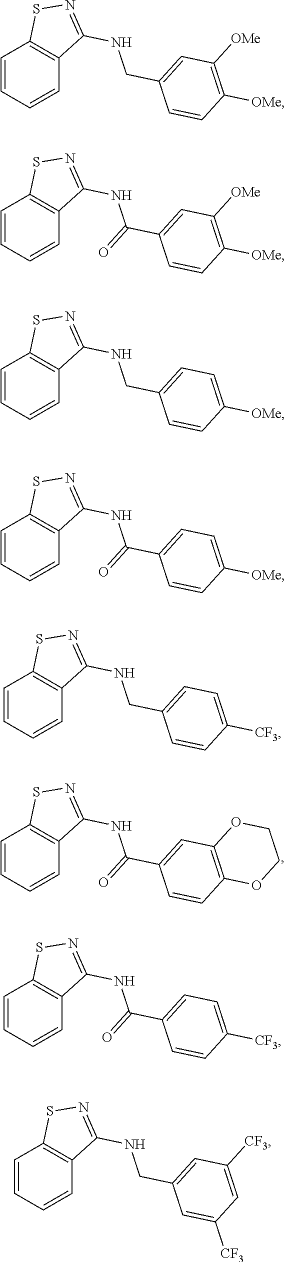

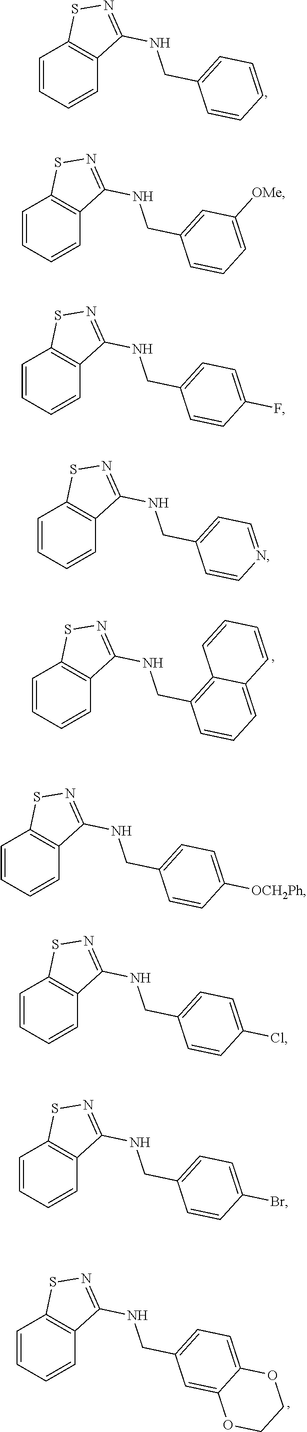

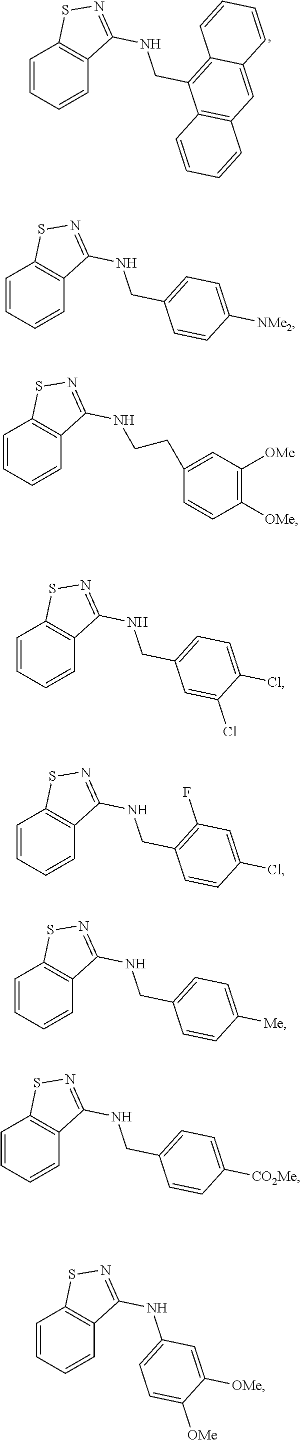

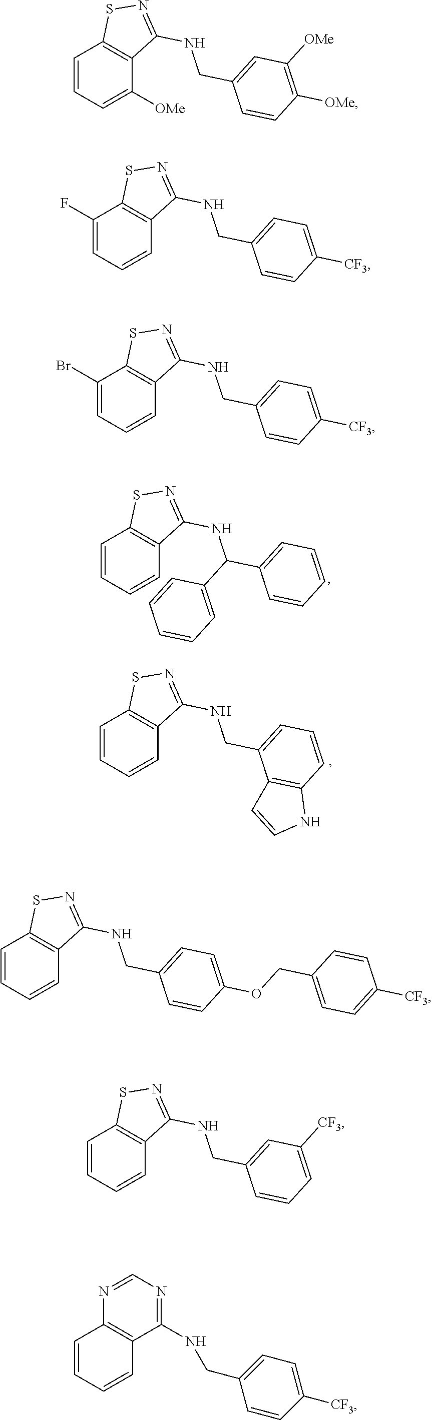

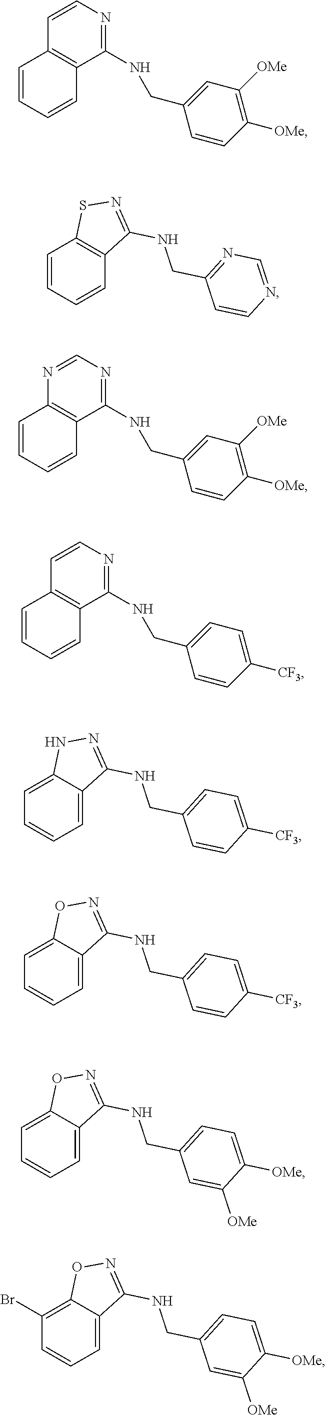

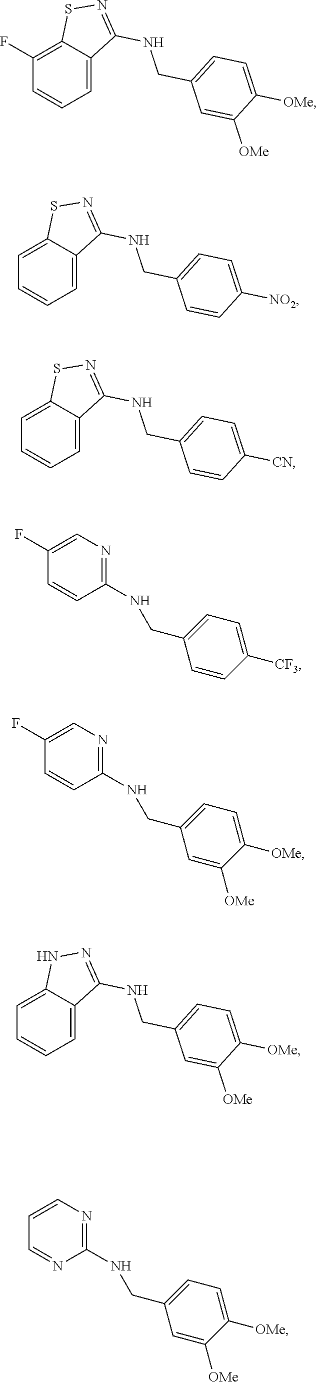

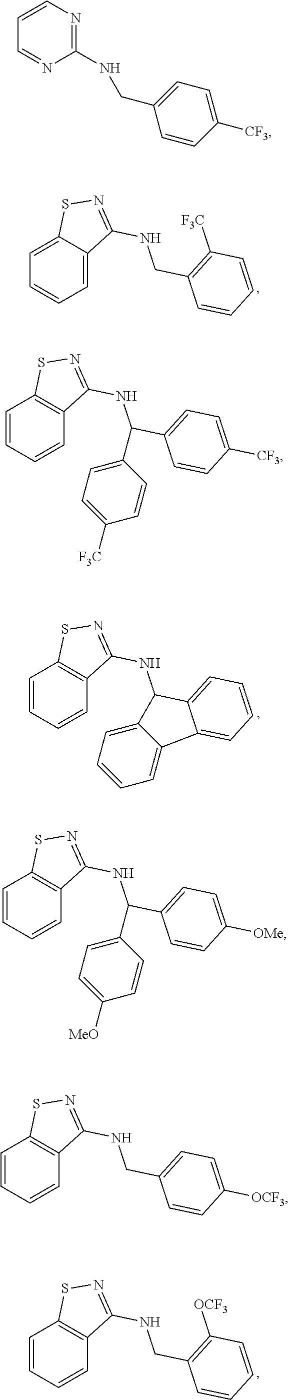

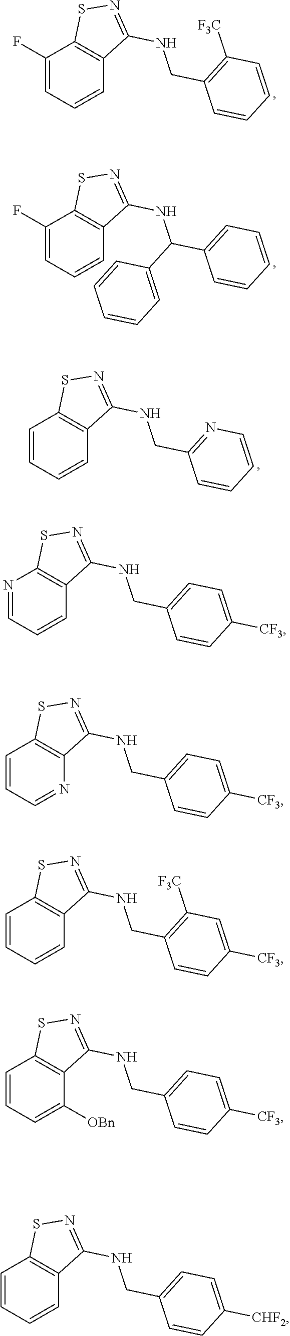

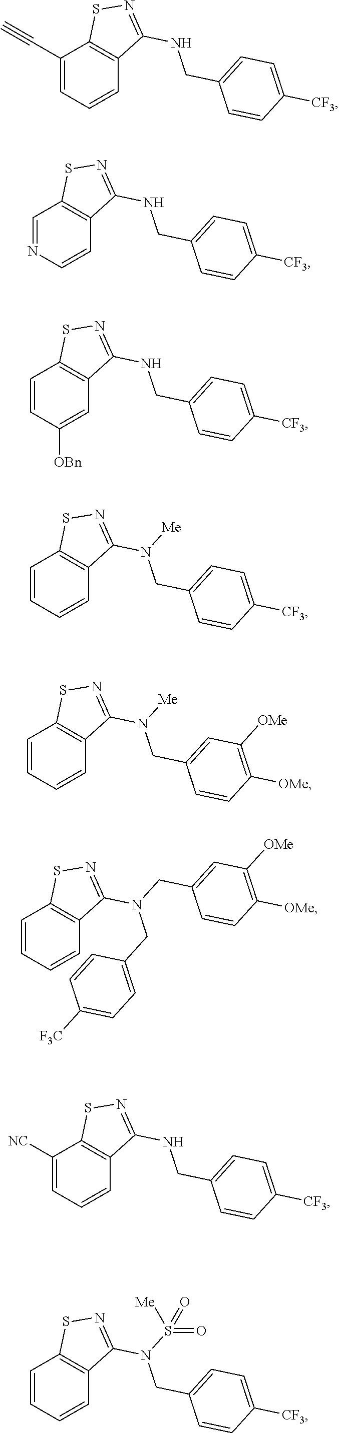

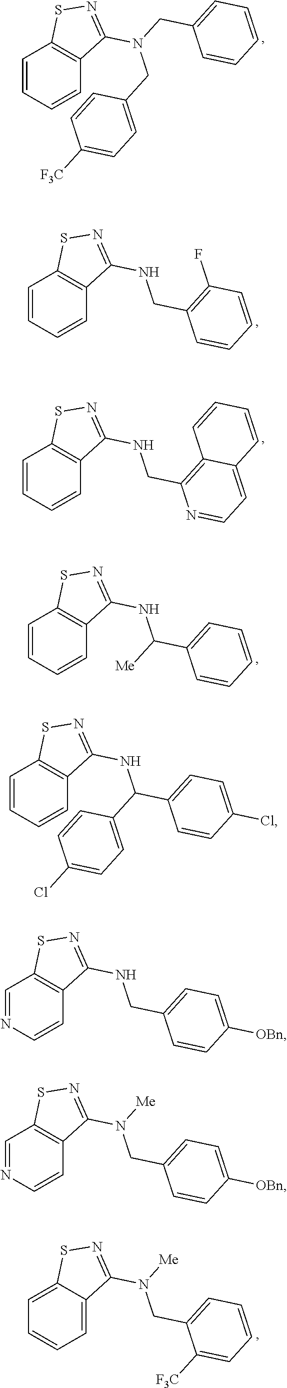

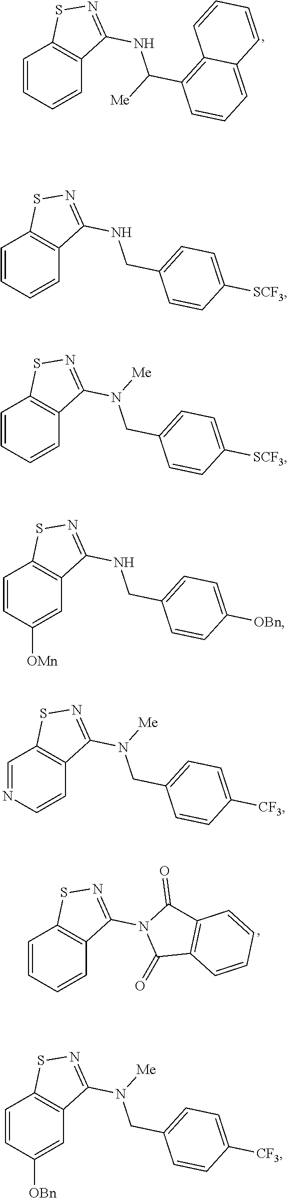

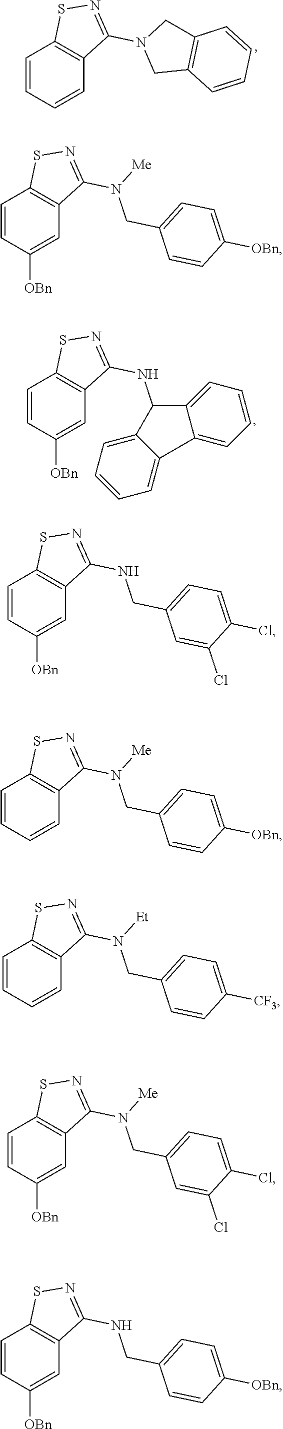

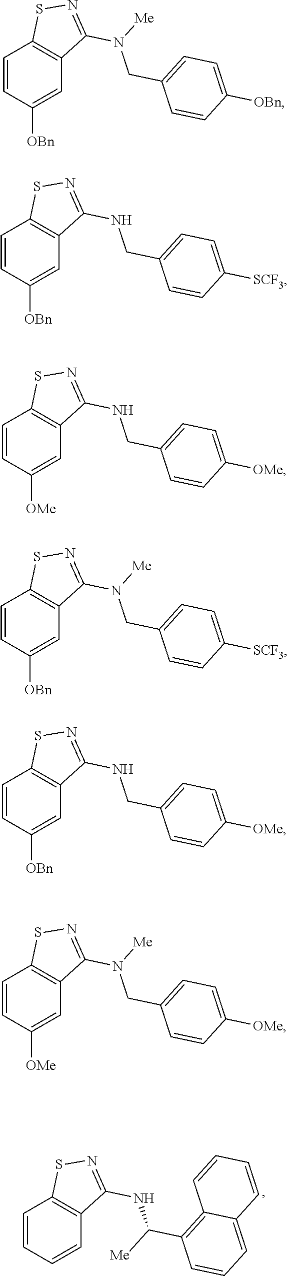

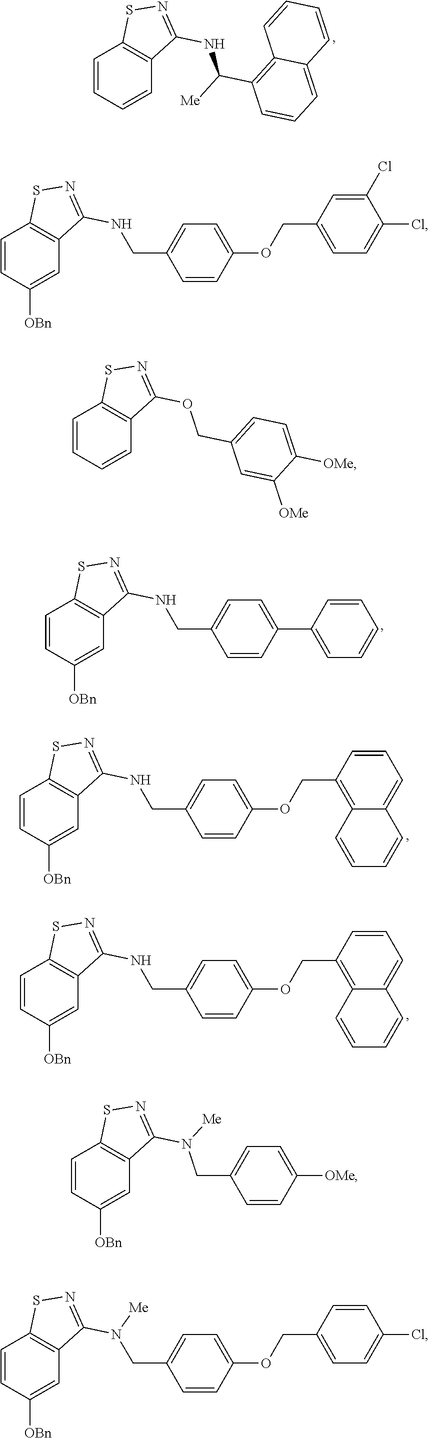

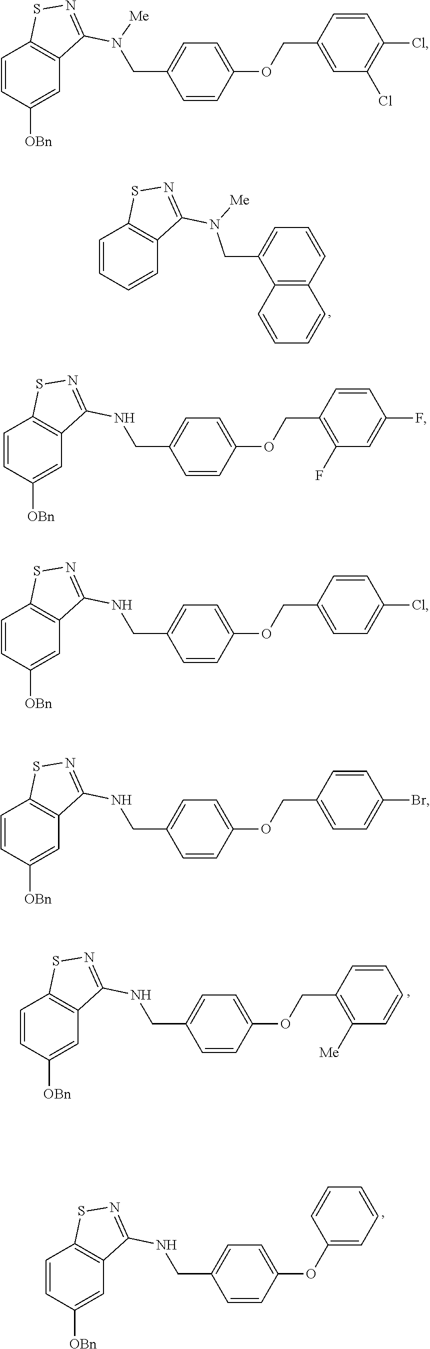

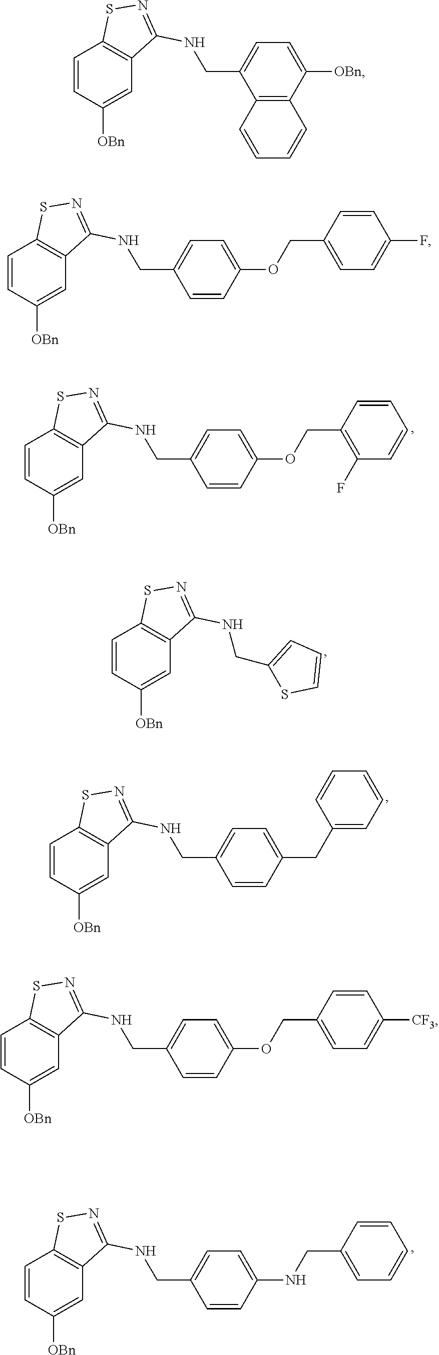

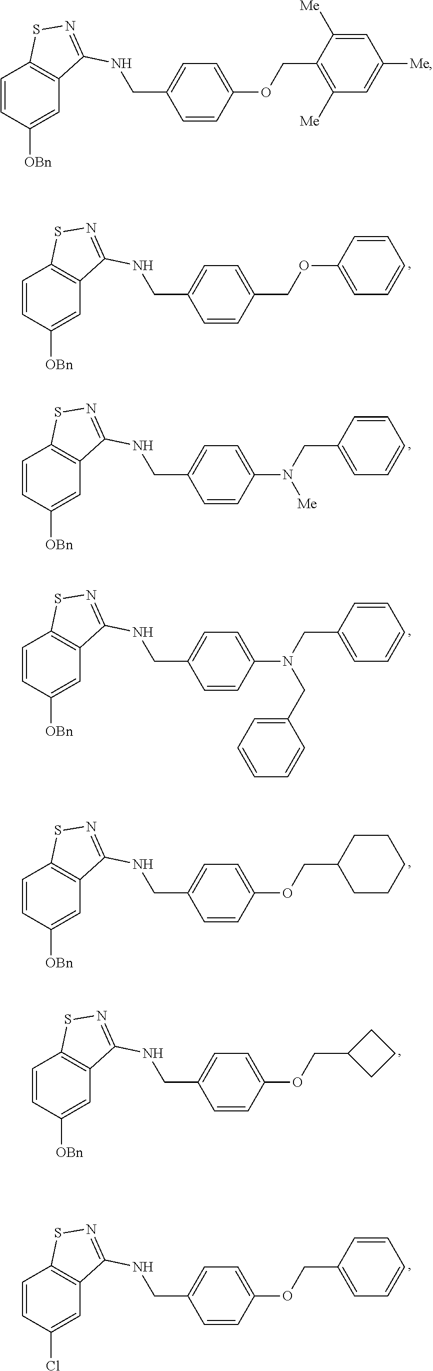

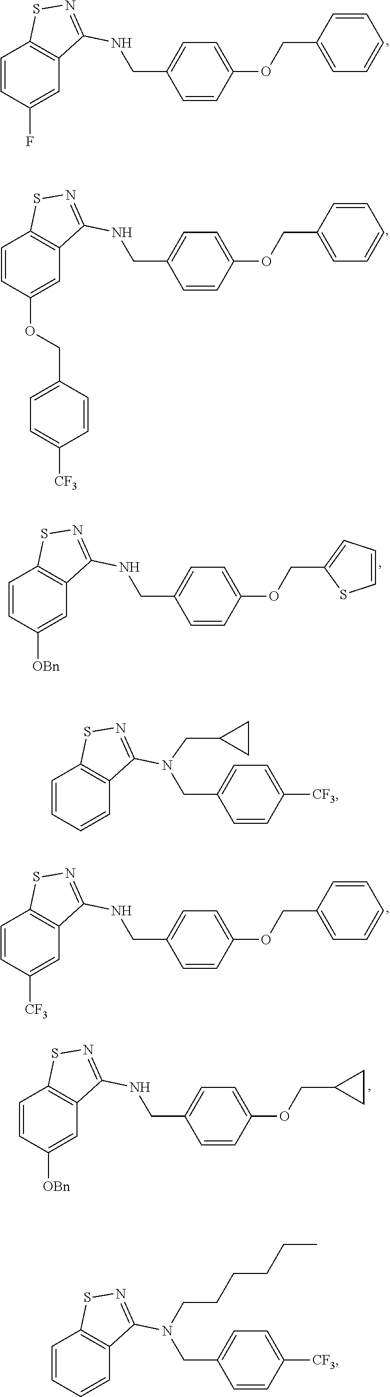

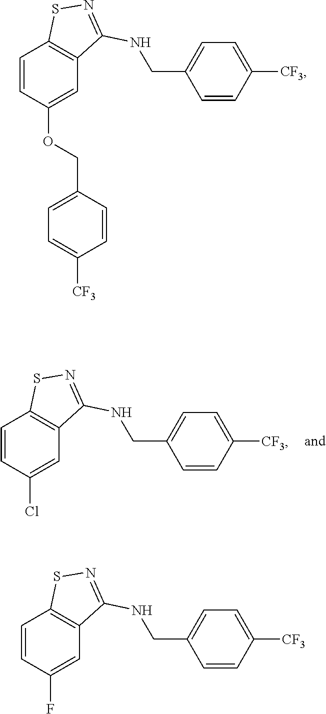 [SMILES: FC(F)(F)c1ccc(CNc2nsc3ccc(OCc4ccc(C(F)(F)F)cc4)cc23)cc1 ; FC(F)(F)c1ccc(CNc2nsc3ccc(Cl)cc23)cc1 ; Fc1ccc2snc(NCc3ccc(C(F)(F)F)cc3)c2c1]

or a pharmaceutically acceptable salt thereof.

In certain aspects, the present disclosure provides pharmaceutical compositions comprising a compound disclosed herein and a pharmaceutically acceptable carrier.

In certain aspects, the present disclosure provides method of treating a proteopathy, comprising administering to a subject in need thereof a compound described herein. The terms “proteopathy”, “proteinopathy” and “protein conformational disorder” refer to a disease or a disorder resulting from the misfolding of one or more proteins. The proteopathy can be a tau-associated neurodegenerative disease selected from Alzheimer's disease, Progressive supranuclear palsy, Corticobasal degeneration, Frontotemporal dementia, Frontotemporal dementia and parkinsonism linked to chromosome 17, Pick's disease, Argyrophilic grain disease, Globular glial tauopathies, Aging-related tau astrogliopathy, Chronic traumatic encephalopathy, Primary age-related tauopathy, Parkinsonism-dementia complex of Guam, Postencephalitic parkinsonism, Atypical Parkinsonism of Guadeloupe, Diffuse neurofilament tangles with calcification, Subacute sclerosing panencephalitis, Lytico-bodig disease, Pantothenate kinase-associated neurodegeneration, and Lipofuscinosis.

In certain embodiments, the proteopathy is a neurodegenerative disease selected from Alzheimer's disease (AD) and AD-related disorders, Parkinson's disease (PD) and PD-related disorders, Huntington's disease and other trinucleotide repeat disorders, Spinocerebellar ataxia (SCA, including SCA 2, SCA 3, SCA 6, SCA 7, SCA 17), Amyotrophic lateral sclerosis, prion disease, Frontotemporal lobar degeneration, Hallervorden-Spatz disease, neuroaxonal dystrophies, familial encephalopathy accompanied by neuroserpin inclusion bodies, Multiple System Atrophy, and Dentatorubralpallidoluysian Atrophy. In further embodiments, the proteopathy is a dementia selected from Alzheimer's disease (AD) and AD-related disorders, Familial Alzheimer's disease, Dementia with Lewy Bodies (dementia accompanied by Lewy bodies), Dementia in Parkinson's disease, Frontotemporal Degeneration, Frontotemporal Dementia, Frontotemporal Dementia with parkinsonism linked to chromosome 17, Primary Progressive Aphasia, Semantic Dementia, Pick's disease, Dementia lacking distinctive histology, Familial British dementia, Familial Danish dementia, dementia pugilistica, and tangle-predominant dementia. In certain embodiments, the proteopathy is Alzheimer's disease. In further embodiments, the proteopathy is Parkinson's Disease.

In certain embodiments, the proteopathy is an amyloidosis or a disease that is caused by or associated with protein aggregation or protein pathology selected from Aβ amyloidosis, AL (light chain) amyloidosis (primary systemic amyloidosis), AH (heavy chain) amyloidosis, AA (secondary) amyloidosis, Aortic medial amyloidosis, apolipoprotein AI amyloidosis (AApoAI), apolipoprotein All amyloidosis (AApoAII), apolipoprotein AIV amyloidosis (AApoAIV), Familial amyloidosis of the Finnish type, Lysozyme amyloidosis, Fibrinogen amyloidosis, Dialysis amyloidosis, Cardiac atrial amyloidosis, Cutaneous lichen amyloidosis, primary cutaneous amyloidosis, Corneal lactoferrin amyloidosis, Lect2 amyloidosis, islet amyloid polypeptide amyloidosis, Hereditary cerebral hemorrhage with amyloidosis, Familial amyloidotic neuropathy, Senile systemic amyloidosis, Mallory bodies, Medullary thyroid carcinoma, Pituitary prolactinoma, Hereditary lattice corneal dystrophy, Odontogenic (Pindborg) tumor amyloid, Seminal vesicle amyloid, Apolipoprotein C2 amyloidosis, Apolipoprotein C3 amyloidosis, Insulin amyloidosis, Galectin-7 amyloidosis (primary localized cutaneous amyloidosis), Corneodesmosin amyloidosis, Enfuvirtide amyloidosis, Cerebral β-amyloid angiopathy, Retinal ganglion cell degeneration in glaucoma, Alexander disease, Pelizaeus-Merzbacher disease, Seipinopathies, Serpinopathies, Inclusion body myositis/myopathy, Cataracts, Retinitis pigmentosa with rhodopsin mutations, Pulmonary alveolar proteinosis, Type II diabetes, Cystic fibrosis, Sickle cell disease, neuronal intranuclear hyaline inclusion disease, and transthyretine-associated cerebral amyloidosis.

In certain embodiments, the proteopathy is a TDP-43 proteinopathy selected from amyotrophic lateral sclerosis, frontotemporal lobar degeneration, limbic-predominant age-related TDP-43 encephalopathy, and Perry syndrome.

In certain embodiments, the proteopathy is a synucleinopathy selected from diseases with Lewy bodies, Parkinson disease, Parkinson-plus syndrome, multiple systemic atrophy, Shy-Drager syndrome, MSA-P (striatonigral degeneration), and olivopontocerebellar atrophy.

In certain embodiments, a compound described herein is administered intravenously. In further embodiments, a compound described herein is administered orally.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

The terms “tauopathy” and “tau-associated neurodegenerative diseases” refer to a disease characterized (clinically, biochemically, and morphologically) by abnormal metabolism, aggregation, or misfolding of microtubule-associated protein tau (MAPT) protein and could lead to intracellular accumulation and formation of neurofibrillary tangles.

The terms “proteopathy”, “proteinopathy” and “protein conformational disorder” refer to a disease or a disorder resulting from the misfolding of one or more proteins.

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen atoms in a given structure with the a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen atoms in a given structure with the substituents mentioned above. More preferably, one to three hydrogen atoms are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

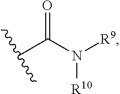

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

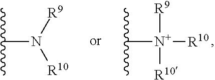

wherein R⁹, R¹⁰, and R¹⁰, each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein includes substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings, wherein at least one of the rings is aromatic, e.g., the other cyclic rings can be cycloalkyls, cycloalkenyls, cycloalkynyls, aryls, heteroaryls, and/or heterocyclyls, and wherein the point of attachment is on the aryl ring; for such polycycles, the ring size indicated for the aryl designate the number of carbon atoms in the ring having the point of attachment.

Aryl groups include benzene, naphthalene, phenanthrene, phenol, aniline, and the like.

The term “carbamate” is art-recognized and refers to a group

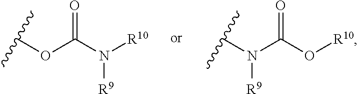

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle.

Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. Heteroaryl groups include, for example, pyrrole, furan, thiophene, imidazole, oxazole, thiazole, pyrazole, pyridine, pyrazine, pyridazine, and pyrimidine, and the like. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls, and wherein the point of attachment is on the heteroaryl ring or an aryl ring fused to it; in such instances, the number of ring members designates the number of ring members in the ring having the point of attachment and any aryl or heteroaryl rings fused to it. That is, for bicyclic heteroaryl groups wherein one ring is an aryl ring (e.g., indolyl, quinolinyl, carbazolyl, and the like), the point of attachment can be on either ring, i.e., either the ring bearing a heteroatom (e.g., 2-indolyl) or the ring that does not contain a heteroatom (e.g., 5-indolyl).

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

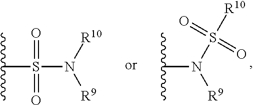

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

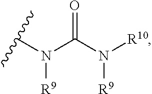

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

“Pharmaceutically acceptable salt” or “salt” is used herein to refer to an acid addition salt or a basic addition salt which is suitable for or compatible with the treatment of patients.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

### EXAMPLES

The invention now being generally described, it will be more readily understood by reference to the following examples which are included merely for purposes of illustration of certain aspects and embodiments of the present invention, and are not intended to limit the invention.

### Example 1. Synthetic Procedures

Procedure A: Benzonitrile (12.4 mmol, 1.5 g) and sodium sulfide (12.4 mmol, 0.97 g) was dissolved in 20 mL DMSO and heated at 70° C. overnight. The mixture was placed in an ice-water bath and treated with concentrated aqueous NH₄OH (16.8 mL) and aqueous NaOCl (16.8 mL). The reaction mixture was allowed to warm to room temperature and stirred for 5 hours. The mixture was diluted with ethyl acetate and washed with water. The organic layer was dried over Na₂SO₄ and concentrated and purified by column chromatography. [1]

Procedure B: To a mixture of Cu(OAc)₂ (10 mol %) and tert-BuOK (1 eq.) in anhydrous toluene, the amine (1 eq.) and alcohol (1.5 eq.) were added successively. After 2 days at 130° C., the resulting mixture was hydrolyzed with Sat. NH₄Cl. The mixture was extracted with EtOAc and washed with brine. The organic phase was dried and the solvent was evaporated. The crude mixture was purified by chromatography (EtOAc/Hexane).[2]

Procedure C: Benzoic acid (1 eq.) was stirred in thionyl chloride (0.1 eq., 3 mL) at 70° C. overnight. Then thionyl chloride was removed under vacuum, and the resulting residue was dissolved in THF, then 3-amino benzoisothiazole (0.9 eq.) and triethylamine (2 eq.) were added at room temperature. When the reaction was complete, the solvent was removed and the residue was purified by column chromatography.

Procedure D: A mixture of 3-chloro benzoisothiazole (0.35 mmol, 60 mg) and amine (10 eq.) in chlorobenzene (20 mL) was heated to 80° C. with stirring for 16 h. Cooled to room temperature, the resulting solution was partitioned between water and EtOAc. The combined extracts were dried over Na₂SO₄ and evaporated in vacuo. The residue was purified using silica gel chromatography (EtOAc/Hexane).[3]

Procedure E: To a THF solution of 3-amino-benzoisothiazole (1 eq.), benzyl bromide (2.2 eq.), Cs₂CO₃ (2.4 eq.) and Et₃N (2.4 eq.) were added. The resulting mixture was heated to 70° C. overnight. The reaction mixture was cooled to room temperature and saturated solution of NH₄Cl was added. The crude product was extracted by EtOAc and purified by column chromatography on silicon gel (EtOAc/Hexane).[4]

Procedure F: To a solution of 3-amino benzoisothiazole (0.333 mmol) in CH₃CN was added LiI (5 mg, cat.), K₂CO₃ (1.31 mmol) and benzhydryl bromide (0.4 mmol). The mixture was stirred at reflux temperature overnight. The solvent was evaporated under reduced pressure, and the residue was added with EtOAc and H₂O. The aqueous layer was extracted with EtOAc and the combined organic layer was dried, filtered and the solvent was removed under reduced pressure. The crude product was purified by flash chromatography (EtOAc/Hexane). [5]

Procedure G: Around bottom flask was charged with indazole (0.333 mmol) and DMF (8 mL). Next AcOH (0.475 mL) was added followed by benzaldehyde (3.33 mmol). The reaction was heated in a 50° C. oil bath for 1 hour. The mixture was cooled to 22° C. over 30 mins, then NaCNBH₃ (3.33 mmol) was added slowly and the mixture was allowed to stir for 16 hours at room temperature. The reaction was diluted in EtOAc and the organic solution was washed with water followed by brine, then dried over Na₂SO₄, concentrated under reduced pressure and purified by column chromatography (EtOAc/Hexane).

Procedure H: To a solution of 9-bromofluorene (1 eq.) in 10 mL propanol was added 3-amino benzoisothiazole (1 eq.) and NaOAc (1.2 eq.). The mixture was refluxed overnight and concentrated. The residue was dissolved in water and extracted with EtOAc. The organic phase was evaporated and purified by silica gel column chromatography (EtOAc).[6]

Procedure I: NaH (2 eq., 60%) and alkyl halide (2 eq.) were added to a solution of JMM005 (1 eq.) in dry THE at room temperature. The reaction was stirred overnight. The reaction mixture was filtered through celite and concentrated in vacuo. The crude residue was purified by column chromatography on silicon gel (EtOAc/Hexane).

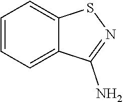

### Procedure a (31% Yield)

¹H NMR (400 MHz, CDCl₃, δ): 7.80 (dt, J=8, 0.8 Hz, 1H), 7.70 (dt, J=8.4, 0.8 Hz, 1H), 7.49 (ddd, J=7, 7.2, 1.2 Hz, 1H), 7.37 (ddd, J=8, 6.8, 1.2 Hz, 1H), 5.03 (brs, 2H); ¹³C NMR (100 MHz, CDCl₃, δ: 158.7, 151.8, 128.1, 126.4, 124.1, 121.7, 120.4;

### JMM001

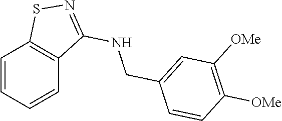 [SMILES: COc1ccc(CNc2nsc3ccccc23)cc1OC]

### Procedure B (41% Yield)

¹H NMR (400 MHz, CDCl₃, δ): 7.80 (d, J=8 Hz, 1H), 7.64 (d, J=8 Hz, 1H), 7.49 (m, 1H), 7.34 (m, 1H), 7.01-6.97 (m, 2H), 6.86 (d, J=8 Hz, 1H), 5.09 (m, 1H), 4.72 (d, J=4.8 Hz, 2H), 3.89 (s, 3H), 3.88 (s, 3H); ¹³C NMR (100 MHz, CDCl₃, δ: 158.7, 151.7, 149.2, 148.6, 131.5, 128.1, 126.3, 123.9, 121.0, 120.5, 120.4, 111.6, 111.3, 56.0, 55.9, 47.3.

### JMM002

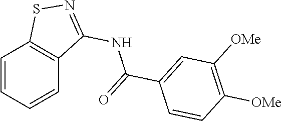 [SMILES: COc1ccc(C(=O)Nc2nsc3ccccc23)cc1OC]

### Procedure C (32% Yield)

¹H NMR (400 MHz, CDCl₃, δ): 8.95 (s, 1H), 8.15 (d, J=8.4 Hz, 1H), 7.87 (d, J=8 Hz, 1H), 7.58-7.52 (m, 3H), 7.43 (ddd, J=8.2, 7.2, 1.2 Hz, 1H), 6.95 (d, J=9.2 Hz, 1H), 3.97 (s, 3H), 3.96 (s, 3H); ¹³C NMR (100 MHz, CDCl₃, □:165.3, 153.2, 152.9, 152.8, 149.3, 128.7, 128.3, 125.8, 125.7, 124.7, 120.8, 119.9, 111.0, 110.5, 56.14, 56.11.

### JMM003

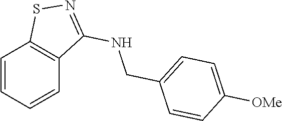 [SMILES: COc1ccc(CNc2nsc3ccccc23)cc1]

### Procedure B (45% Yield)

¹H NMR (400 MHz, CDCl₃, δ): 7.79 (d, J=8 Hz, 1H), 7.62 (d, J=8 Hz, 1H), 7.48 (ddd, J=8.4, 6.8, 0.8 Hz, 1H), 7.37 (d, J=8.8 Hz, 2H), 7.33 (ddd, J=8, 7.2, 1.2 Hz, 1H), 6.90 (d, J=8.4 Hz, 1H), 5.07 (m, 1H), 4.71 (d, J=5.2 Hz, 1H), 3.81 (s, 3H); ¹³C NMR (100 MHz, CDCl₃, δ: 159.1, 158.7, 151.7, 131.0, 129.4, 128.0, 126.4, 123.8, 121.0, 120.4, 114.1, 55.3, 46.8.

### JMM004

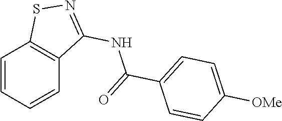 [SMILES: COc1ccc(C(=O)Nc2nsc3ccccc23)cc1]

### Procedure C (32% Yield)

¹H NMR (400 MHz, CDCl₃, δ): 8.89 (s, 1H), 8.15 (d, J=9.2 Hz, 1H), 7.96 (d, J=8.8 Hz, 2H), 7.87 (d, J=8.4 Hz, 1H), 7.54 (ddd, J=8.2, 6.8, 1.2 Hz, 1H), 7.43 (ddd, J=8.2, 6.8, 1.2 Hz, 1H), 7.01 (d, J=8.8 Hz, 2H), 3.90 (s, 3H); ¹³C NMR (100 MHz, CDCl₃, δ: 165.2, 163.2, 153.2, 152.9, 129.7, 128.7, 128.2, 125.7, 125.5, 124.7, 119.9, 114.1, 55.5;

### JMM005

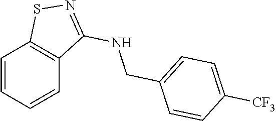 [SMILES: FC(F)(F)c1ccc(CNc2nsc3ccccc23)cc1]

### Procedure B (45% Yield)

¹H NMR (400 MHz, CDCl₃, δ): 7.81 (d, J=8.4 Hz, 1H), 7.67 (d, J=8 Hz, 1H), 7.61 (d, J=8.4 Hz, 2H), 7.55 (d, J=8 Hz, 2H), 7.51 (ddd, J=8, 7.2, 1.2 Hz, 1H), 7.37 (ddd, J=8, 7.2, 0.8 Hz, 1H), 5.26 (m, 1H), 4.86 (d, J=6 Hz, 1H); ¹³C NMR (100 MHz, CDCl₃, δ: 158.4, 151.9, 143.2, 129.7 (q, J=32.2 Hz), 128.2, 128.0, 126.2, 125.6 (q, J=3.8 Hz), 124.2 (q, J=270.3 Hz), 124.0, 120.9, 120.5, 46.7; ¹⁹F NMR (376 MHz, CDCl₃, δ): −62.4;

### JMM006

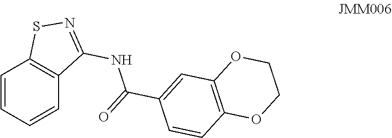 [SMILES: O=C(Nc1nsc2ccccc12)c1ccc2c(c1)OCCO2]

### Procedure C (37% Yield)

¹H NMR (400 MHz, DMSO-d⁶, δ): 10.9 (s, 1H), 8.14 (d, J=8 Hz, 1H), 7.9 (d, J=8 Hz, 1H), 7.60-7.55 (m, 3H), 7.44 (ddd, J=8.2, 6.8, 1.2 Hz, 1H), 6.98 (d, J=8 Hz, 1H), 4.32-4.26 (m, 4H); ¹³C NMR (100 MHz, DMSO-d⁶, δ: 165.6, 154.3, 152.1, 147.4, 143.5, 130.1, 128.6, 126.5, 125.3, 125.2, 122.2, 121.2, 117.7, 117.5, 64.9, 64.5;

### JMM007

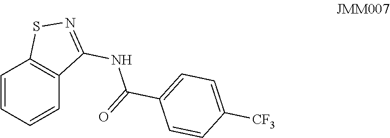 [SMILES: O=C(Nc1nsc2ccccc12)c1ccc(C(F)(F)F)cc1]

### Procedure C (27% Yield)

¹H NMR (400 MHz, CDCl₃, δ): 8.15-8.1 (m, 3H), 7.9 (d, J=8 Hz, 1H), 7.8 (d, J=8.8 Hz, 2H), 7.57 (d, J=7.6 Hz, 1H), 7.46 (d, J=8 Hz, 1H); ¹³C NMR (100 MHz, CDCl₃, δ: 164.5, 157.6, 153.0, 136.6, 134.3 (q, J=32.7 Hz), 128.5, 128.2, 126.0 (q, J=3.9 Hz), 125.3, 124.9, 122.4 (q, J=270.7 Hz), 120.0, 115.1; ¹⁹F NMR (376 MHz, CDCl₃, δ): −63.0;

### JMM008

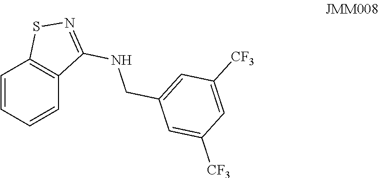 [SMILES: FC(F)(F)c1cc(CNc2nsc3ccccc23)cc(C(F)(F)F)c1]

### Procedure B (17% Yield)

¹H NMR (400 MHz, CDCl₃, δ): 7.9 (s, 2H), 7.82-7.79 (m, 2H), 7.73 (d, J=8 Hz, 1H), 7.52 (ddd, J=8.2, 6.8, 1.2 Hz, 1H), 7.39 (ddd, J=8, 6.8, 1.2 Hz, 1H), 5.48 (s, 1H), 4.92 (s, 2H); ¹³C NMR (100 MHz, CDCl₃, δ: 158.0, 151.9, 141.9, 131.8 (q, J=33 Hz), 128.4, 127.9 (m), 126.0, 123.3 (q, J=271 Hz), 124.2, 121.4 (m), 121.0, 120.6, 46.4; ¹⁹F NMR (376 MHz, CDCl₃, δ): −62.8.

### JMM009

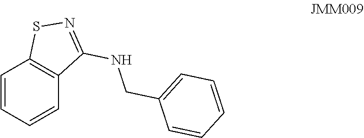 [SMILES: c1ccc(CNc2nsc3ccccc23)cc1]

### Procedure B (63% Yield)

¹H NMR (400 MHz, CDCl₃, δ): 7.79 (d, J=8 Hz, 1H), 7.69 (d, J=8.4 Hz, 1H), 7.51-7.47 (m, 1H), 7.44 (d, J=6.8 Hz, 2H), 7.38-7.28 (m, 4H), 5.42 (s, 1H), 4.79 (s, 2H); ¹³C NMR (100 MHz, CDCl₃, δ: 158.6, 151.4, 138.8, 128.7, 128.3, 128.1, 127.6, 126.1, 124.0, 121.2, 120.5, 47.4; ¹⁹F NMR (376 MHz, CDCl₃, δ): −62.8.

### JMM010

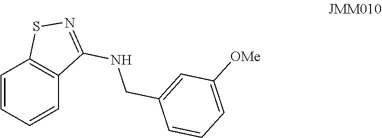 [SMILES: COc1cccc(CNc2nsc3ccccc23)c1]

### Procedure B (30% Yield)

¹H NMR (400 MHz, CDCl₃, δ): 7.79-7.74 (m, 2H), 7.5 (ddd, J=8.4, 6.8, 0.8 Hz, 1H), 7.35 (ddd, J=8.4, 6.8, 0.8 Hz, 1H), 7.28-7.24 (m, 1H), 7.04-7.0 (m, 2H), 6.83 (dd, J=5.6, 2.8 Hz, 1H), 5.65 (s, 1H), 4.77 (s, 2H), 3.79 (s, 3H); ¹³C NMR (100 MHz, CDCl₃, δ: 159.9, 158.5, 151.0, 140.2, 129.7, 128.6, 125.9, 124.2, 121.5, 120.4, 120.3, 113.7, 113.0, 55.3, 47.3;

### JMM011

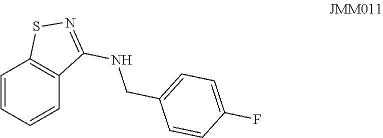 [SMILES: Fc1ccc(CNc2nsc3ccccc23)cc1]

### Procedure B (27%)

¹H NMR (400 MHz, CDCl₃, δ): 7.79 (d, J=8 Hz, 1H), 7.67 (d, J=8 Hz, 1H), 7.50 (ddd, J=8.4, 6.8, 0.8 Hz, 1H), 7.42-7.39 (m, 2H), 7.35 (J=8, 6.8, 0.8 Hz, 1H), 7.06-7.01 (m, 2H), 5.28 (s, 1H), 4.75 (s, 2H); ¹³C NMR (100 MHz, CDCl₃, δ: 162.2 (d, J=244 Hz), 158.5, 151.6, 134.7 (d, J=3.2 Hz), 129.6 (d, J=8.1 Hz), 128.2, 126.2, 124.0, 121.0, 120.5, 115.5 (d, J=21.2 Hz), 46.6; ¹⁹F NMR (376 MHz, CDCl₃, δ): −115.1.

### JMM012

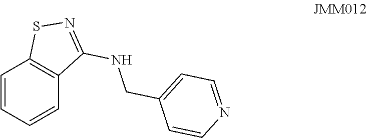 [SMILES: c1ccc2c(NCc3ccncc3)nsc2c1]

### Procedure B (24%)

¹H NMR (400 MHz, CDCl₃, δ): 8.55 (m, 2H), 7.80 (d, J=8.4 Hz, 1H), 7.73 (d, J=8 Hz, 1H), 7.51 (ddd, J=8.2, 6.8, 1.2 Hz, 1H), 7.39-7.35 (m, 3H), 5.53 (s, 1H), 4.82 (d, J=5.6, 2H); ¹³C NMR (100 MHz, CDCl₃, δ: 158.3, 151.9, 149.4, 149.1, 128.2, 126.1, 124.1, 122.6, 121.0, 120.5; 45.9;

### JMM013

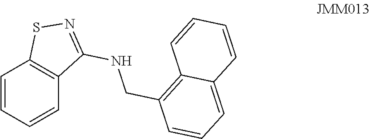 [SMILES: c1ccc2c(CNc3nsc4ccccc34)cccc2c1]

### Procedure B (15%)

¹H NMR (400 MHz, CDCl₃, δ): 8.17-8.14 (m, 1H), 7.92-7.88 (m, 1H), 7.86 (d, J=8 Hz, 1H), 7.8 (d, J=8.4 Hz, 1H), 7.6-7.57 (m, 2H), 7.53-7.45 (m, 4H), 7.30 (ddd, J=8, 6.8, 1.2 Hz, 1H), 5.23 (s, 2H); ¹³C NMR (100 MHz, CDCl₃, δ: 158.6, 151.5, 134.0, 133.9, 131.7, 128.8, 128.7, 128.2, 126.9, 126.6, 126.1, 126.0, 125.5, 123.9, 123.8, 121.2, 120.5, 45.6;

### JMM014

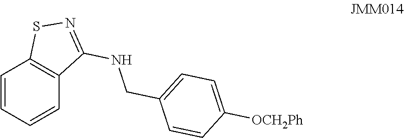

### Procedure B (10%)

¹H NMR (400 MHz, CDCl₃, δ): 7.78 (d, J=8 Hz, 1H), 7.68 (d, J=8 Hz, 1H), 7.49 (ddd, J=8.2, 6.8, 1.2 Hz, 1H), 7.44-7.40 (m, 3H), 7.39-7.32 (m, 5H), 6.96 (d, J=8.8 Hz, 2H), 5.07 (s, 2H), 4.72 (s, 2H); ¹³C NMR (100 MHz, CDCl₃, δ: 158.6, 158.3, 137.0, 131.1, 129.5, 128.8, 128.6, 128.3, 128.0, 127.4, 126.1, 124.0, 121.2, 120.4, 115.1, 70.1, 46.9;

### JMM015

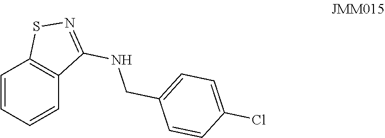 [SMILES: Clc1ccc(CNc2nsc3ccccc23)cc1]

### Procedure B (29%)

¹H NMR (400 MHz, CDCl₃, δ): 7.79 (d, J=8.4 Hz, 1H), 7.69 (d, J=8.4 Hz, 1H), 7.50 (ddd, J=8, 7.2, 1.2 Hz), 7.38-7.35 (m, 3H), 7.33-7.30 (m, 2H), 5.38 (brs, 1H), 4.76 (s, 2H); ¹³C NMR (100 MHz, CDCl₃, δ: 158.4, 151.7, 137.5, 133.3, 129.3, 128.8, 128.2, 126.1, 124.0, 121.0, 120.5, 46.6;

### JMM016

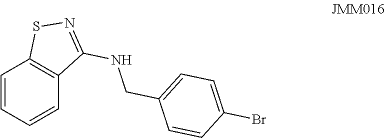 [SMILES: Brc1ccc(CNc2nsc3ccccc23)cc1]

### Procedure B (10%)

¹H NMR (400 MHz, CDCl₃, δ): 7.79 (d, J=8.4 Hz, 1H), 7.68 (d, J=8.4 Hz, 1H), 7.52-7.45 (m, 3H), 7.35 (ddd, J=8, 7.2, 1.2 Hz, 1H), 7.31 (d, J=8.4 Hz, 2H), 5.32 (brs, 1H), 4.74 (s, 2H); ¹³C NMR (100 MHz, CDCl₃, δ: 158.4, 151.6, 138.0, 131.8, 129.6, 128.3, 126.1, 124.0, 121.3, 121.0, 120.5, 46.6;

### JMM017

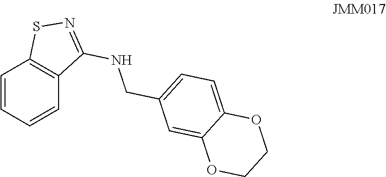 [SMILES: c1ccc2c(NCc3ccc4c(c3)OCCO4)nsc2c1]

### Procedure B (52%)

¹H NMR (400 MHz, CDCl₃, δ): 7.78 (d, J=8 Hz, 1H), 7.64 (d, J=8 Hz, 1H), 7.48 (ddd, J=8.4, 6.8, 0.8 Hz, 1H), 7.32 (ddd, J=8.4, 6.8, 0.8 Hz, 1H), 6.96 (d, J=2.4 Hz, 1H), 6.90 (dd, J=8, 2 Hz, 1H), 6.85 (d, J=8.4 Hz, 1H), 5.17 (brs, 1H), 4.66 (s, 2H), 4.24 (s, 4H); ¹³C NMR (100 MHz, CDCl₃, δ: 158.7, 151.6, 143.6, 143.0, 132.2, 128.1, 126.3, 123.9, 121.1, 121.0, 120.4, 117.4, 117.0, 64.39, 64.37, 46.8;

### JMM018

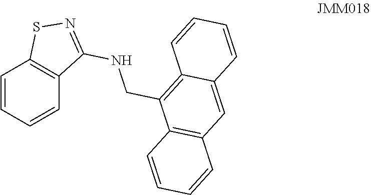 [SMILES: c1ccc2c(CNc3nsc4ccccc34)c3ccccc3cc2c1]

### Procedure B (6%)

¹H NMR (400 MHz, CDCl₃, δ): 8.52 (s, 1H), 8.39 (d, J=8.4 Hz, 2H), 8.07 (d, J=8 Hz, 2H), 7.83 (d, 8 Hz, 1H), 7.57-7.44 (m, 6H), 7.23 (d, J=7.6 Hz, 1H), 5.75 (s, 2H), 5.11 (brs, 1H); ¹³C NMR (100 MHz, CDCl₃, δ: 158.8, 131.6, 130.6, 129.2, 129.0, 128.3, 128.2, 126.7, 126.0, 125.4, 125.3, 124.1, 123.9, 121.2, 120.5, 39.9.

### JMM019

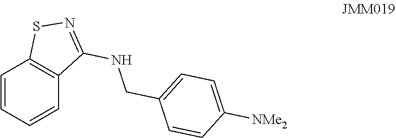 [SMILES: CN(C)c1ccc(CNc2nsc3ccccc23)cc1]

### Procedure B (5%)

¹H NMR (400 MHz, CDCl₃, δ): 7.78 (d, J=8.4 Hz, 1H), 7.60 (d, J=8 Hz, 1H), 7.47 (ddd, J=8.2, 6.8, 0.8 Hz, 1H), 7.35-7.29 (m, 3H), 6.76 (d, J=8.4 Hz, 2H), 5.04 (brs, 1H), 4.67 (d, J=5.2 Hz, 2H), 2.96 (s, 6H); ¹³C NMR (100 MHz, CDCl₃, δ: 158.9, 151.6, 150.1, 129.4, 128.0, 126.4, 124.1, 123.8, 121.0, 120.4, 112.9, 47.0, 40.8;

### JMM020

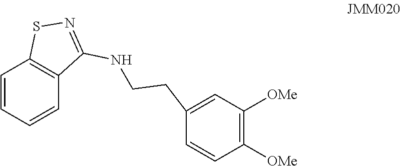 [SMILES: COc1ccc(CCNc2nsc3ccccc23)cc1OC]

### Procedure D (24%)

¹H NMR (400 MHz, CDCl₃, δ): 7.77 (d, J=8 Hz, 1H), 7.54 (d, J=8 Hz, 1H), 7.47 (ddd, J=8.4, 6.8, 0.8 Hz, 1H), 7.31 (ddd, J=8, 6.8, 0.8 Hz, 1H), 6.83-6.77 (m, 2H), 6.75 (J=2 Hz, 1H), 3.86 (s, 3H), 3.85 (t, J=6.8 Hz, 2H), 3.81 (s, 3H), 2.99 (t, J=6.8 Hz, 2H); ¹³C NMR (100 MHz, CDCl₃, δ: 138.1, 131.8, 128.0, 126.4, 123.8, 120.8, 120.75, 120.5, 119.6, 115.0, 112.0, 111.3, 111.0, 55.9, 55.8, 44.1, 35.2.

### JMM021

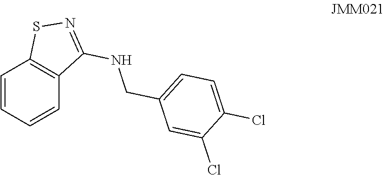 [SMILES: Clc1ccc(CNc2nsc3ccccc23)cc1Cl]

### Procedure B (59%)

¹H NMR (400 MHz, CDCl₃, δ): 7.79 (d, J=8.4 Hz, 1H), 7.66 (d, J=8.4 Hz, 1H), 7.52-7.48 (m, 2H), 7.37 (d, J=8.4 Hz, 1H), 7.35 (ddd, J=8.0, 6.8, 1.2 Hz, 1H), 7.23 (dd, J=8.4, 2.0 Hz, 1H), 5.32 (br s, 1H), 4.72 (d, J=6.0 Hz, 2H); ¹³C NMR (100 MHz, CDCl₃, δ): 158.3, 151.8, 139.5, 132.6, 131.3, 130.5, 129.6, 128.2, 127.1, 126.1, 124.1, 120.9, 120.5, 46.0.

### JMM022

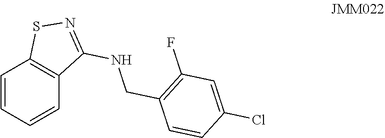 [SMILES: Fc1cc(Cl)ccc1CNc1nsc2ccccc12]

### Procedure B (49%)

¹H NMR (400 MHz, CDCl₃, δ): 10.40 (d, J=0.8 Hz, 1H), 7.80 (dd, J=6.8, 0.8 Hz, 1H), 7.44 (t, J=8 Hz, 1H), 7.22-7.16 (m, 2H), 7.08-7.05 (m, 2H), 5.19 (s, 2H); ¹³C NMR (100 MHz, CDCl₃, δ: 188.2, 160.8, 160.3 (d, J=249.9 Hz), 141.9, 135.6 (d, J=10.3 Hz), 130.5 (d, J=4.6 Hz), 129.9, 125.0 (d, J=3.7 Hz), 123.8, 122.0, 121.3 (d, J=14.5 Hz), 116.6 (d, J=24.5 Hz), 113.5, 64.3 (d, J=3.9 Hz); ¹⁹F NMR (376 MHz, CDCl₃, δ): −115.5.

### JMM023

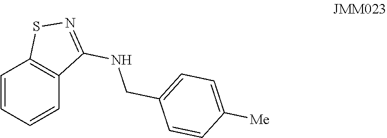 [SMILES: Cc1ccc(CNc2nsc3ccccc23)cc1]

### Procedure B (18%)

¹H NMR (400 MHz, CDCl₃, δ): 7.79 (d, J=8.4 Hz, 1H), 7.66 (d, J=8.4 Hz, 1H), 7.49 (ddd, J=8.4, 6.8, 1.2 Hz, 1H), 7.36-7.32 (m, 3H), 7.17 (d, J=7.6 Hz, 2H), 5.28 (brs, 1H), 4.75 (s, 2H), 2.35 (s, 3H); ¹³C NMR (100 MHz, CDCl₃, δ: 158.7, 151.5, 137.3, 135.8, 129.4, 128.15, 128.10, 126.3, 123.9, 121.1, 120.4, 47.2, 21.1.

### JMM024

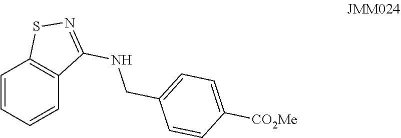 [SMILES: COC(=O)c1ccc(CNc2nsc3ccccc23)cc1]

### Procedure B (3%)

¹H NMR (400 MHz, CDCl₃, δ): 10.11, 8.24 (d, J=8 Hz, 2H), 8.07 (d, J=8.4 Hz, 2H), 7.98-7.95 (m, 2H), 7.53-7.50 (m, 2H), 5.45 (s, 2H), 3.93 (s, 3H); ¹³C NMR (100 MHz, CDCl₃, δ: 191.5, 175.1, 166.6, 165.2, 140.5, 139.4, 134.8, 130.3, 130.2, 130.0, 129.6, 127.8, 66.5, 52.2;

### JMM025

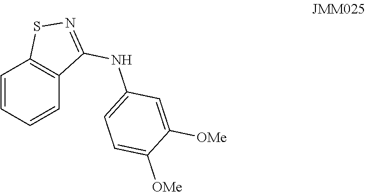 [SMILES: COc1ccc(Nc2nsc3ccccc23)cc1OC]

A mixture of 3-chloro benzoisothiazole (0.41 mmol, 70 mg), Pd(OAc)₂ (5 mol %), Xantphos (10 mol %), Cs₂CO₃ (0.62 mmol, 201 mg) and 3,4-dimethoxy aniline (0.41 mmol, 63 mg) was added 1,4-dioxane under Ar. The reaction mixture was heated at 100° C. overnight and after it cooled to room temperature, filter through the celite. The solution was concentrated and purified on silicon gel column chromatography (EtOAc/Hexane) (9% yield).

¹H NMR (400 MHz, CDCl₃, δ): 7.83 (d, J=8 Hz, 1H), 7.78 (d, J=8 Hz, 1H), 7.53 (ddd, J=8, 7.2, 1.2 Hz, 1H), 7.49 (d, J=2.4 Hz, 1H), 7.40 (ddd, J=8.4, 6.8, 0.8 Hz, 1H), 7.15-7.14 (m, 2H), 6.86 (d, J=8.4 Hz, 1H), 3.92 (s, 3H), 3.87 (s, 3H); ¹³C NMR (100 MHz, CDCl₃, δ: 150.9, 149.4, 134.0, 128.3, 126.8, 124.2, 121.1, 120.5, 120.2, 111.9, 110.8, 110.5, 104.2, 56.3, 55.99;

### JMM026

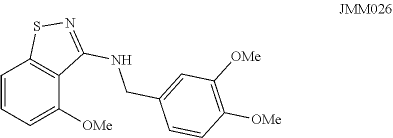 [SMILES: COc1ccc(CNc2nsc3cccc(OC)c23)cc1OC]

### Procedure A (24%)

¹H NMR (400 MHz, CDCl₃, δ): 7.37-7.28 (m, 2H), 6.99 (d, J=2 Hz, 1H), 6.98-6.95 (m, 1H), 6.85 (d, J=8 Hz, 1H), 6.66 (dd, J=6.8, 0.8 Hz, 1H), 6.58 (brs, 1H), 4.68 (s, 2H), 3.93 (s, 3H), 3.88 (s, 3H), 3.87 (s, 3H); ¹³C NMR (100 MHz, CDCl₃, δ: 159.4, 156.7, 154.5, 149.1, 148.3, 132.2, 129.3, 119.8, 116.5, 112.8, 111.2, 111.18, 103.9, 56.0, 55.9, 55.6, 46.8.

### JMM027

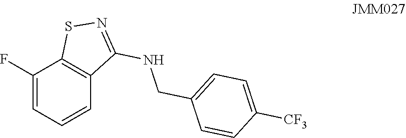 [SMILES: Fc1cccc2c(NCc3ccc(C(F)(F)F)cc3)nsc12]

### Procedure E (5%)

¹H NMR (400 MHz, CDCl₃, δ): 7.61 (d, J=8.4 Hz, 2H), 7.54 (d, J=8.4 Hz, 2H), 7.46 (d, J=8 Hz, 1H), 7.34 (td, J=8, 4.4 Hz, 1H), 7.18 (ddd, J=9.4, 8, 0.8 Hz, 1H), 5.28 (brs, 1H), 4.85 (s, 2H); ¹³C NMR (100 MHz, CDCl₃, δ: 158.3, 156.0 (d, J=246.6 Hz), 142.9, 139.3, 129.8 (q, J=32.2 Hz), 129.85 (d, J=5 Hz), 128.0, 126.1 (d, J=6 Hz), 124.1 (q, J=270.3 Hz), 125.6 (q, J=3.8 Hz), 116.7 (d, J=3.9 Hz), 113.4 (d, J=18.5 Hz), 46.8; ¹⁹F NMR (376 MHz, CDCl₃, δ): −62.5, −114.3.

### JMM028

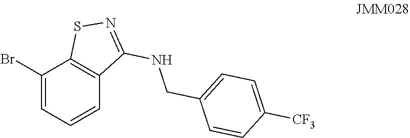 [SMILES: FC(F)(F)c1ccc(CNc2nsc3c(Br)cccc23)cc1]

### Procedure E (13%)

¹H NMR (400 MHz, CDCl₃, δ): 7.64-7.59 (m, 4H), 7.53 (d, J=8.4 Hz, 2H), 7.27-7.23 (m, 1H), 5.25 (brs, 1H), 4.83 (d, J=4.8 Hz, 2H); ¹³C NMR (100 MHz, CDCl₃, δ: 158.7, 153.8, 142.9, 130.9, 129.8 (q, J=32.2 Hz), 128.0, 127.4, 125.6 (q, J=3.6 Hz), 124.1 (q, J=270.5 Hz), 121.4, 119.8, 114.1, 46.6; ¹⁹F NMR (376 MHz, CDCl₃, δ): −62.4.

### JMM029

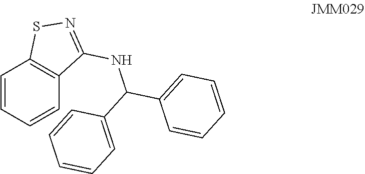 [SMILES: c1ccc(C(Nc2nsc3ccccc23)c2ccccc2)cc1]

### Procedure F (37%)

¹H NMR (400 MHz, CDCl₃, δ): 7.78 (d, J=8 Hz, 1H), 7.71 (d, J=8.4 Hz, 1H), 7.49 (ddd, J=8, 6.8, 0.8 Hz, 1H), 7.40-7.32 (m, 9H), 7.30-7.25 (m, 2H), 6.42 (d, J=5.6 Hz, 1H), 5.44 (d, J=6 Hz, 1H); ¹³C NMR (100 MHz, CDCl₃, δ: 157.5, 151.8, 142.5, 128.7, 128.0, 127.5, 127.4, 126.3, 123.9, 120.9, 120.4, 60.8.

### JMM030

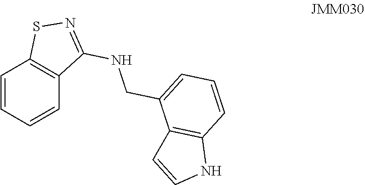 [SMILES: c1cc(CNc2nsc3ccccc23)c2cc[nH]c2c1]

### Procedure B (9%)

¹H NMR (400 MHz, CD₃OD, δ): 7.97 (d, J=8 Hz, 1H), 7.79 (d, J=8 Hz, 1H), 7.48 (ddd, J=8.2, 6.8, 1.2 Hz, 1H), 7.34-7.27 (m, 2H), 7.20 (d, J=3.2 Hz, 1H), 7.05-7.03 (m, 2H), 6.54 (dd, J=3.2, 1.2 Hz, 1H), 4.96 (s, 2H); ¹³C NMR (100 MHz, CD₃OD, δ: 159.9, 150.8, 136.4, 130.0, 128.1, 126.7, 126.5, 124.0, 123.6, 122.0, 120.8, 119.7, 117.5, 110.0, 99.1, 44.8.

### JMM031

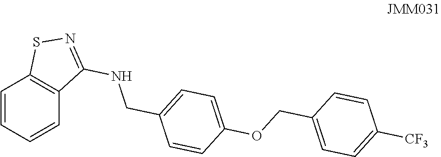 [SMILES: FC(F)(F)c1ccc(COc2ccc(CNc3nsc4ccccc34)cc2)cc1]

### Procedure B (19%)

¹H NMR (400 MHz, CDCl₃, δ): 7.78 (d, J=8 Hz, 1H), 7.65-7.62 (m, 3H), 7.55 (d, J=8 Hz, 2H), 7.48 (ddd, J=8.4, 6.8, 0.8 Hz, 1H), 7.37 (d, J=8.4 Hz, 2H), 7.32 (ddd, J=8, 6.8, 0.8 Hz, 1H), 6.95 (d, J=8.8 Hz, 2H), 5.12 (s, 2H), 4.71 (s, 2H); ¹³C NMR (100 MHz, CDCl₃, δ: 158.7, 157.9, 151.7, 141.1, 131.8, 130.1 (q, J=32.2 Hz), 129.9, 129.5, 128.1, 127.3, 126.4, 125.6 (q, J=3.8 Hz), 124.1 (q, J=270.4 Hz), 123.9, 121.0, 120.4, 115.0, 69.2, 46.7; ¹⁹F NMR (376 MHz, CDCl₃, δ): −62.5.

### JMM032

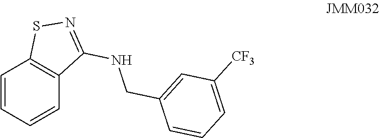 [SMILES: FC(F)(F)c1cccc(CNc2nsc3ccccc23)c1]

### Procedure B (11%)

¹H NMR (400 MHz, CDCl₃, δ): 7.80 (d, J=8 Hz, 1H), 7.81-7.79 (m, 2H), 7.63 (d, J=7.6 Hz, 1H), 7.55 (d, J=7.6 Hz, 1H), 7.52-7.45 (m, 2H), 7.36 (ddd, J=8, 7.2, 1.2 Hz, 1H), 5.28 (brs, 1H), 4.85 (s, 2H); ¹³C NMR (100 MHz, CDCl₃, δ: 158.4, 151.8, 140.1, 131.3, 131.0 (q, J=31.8 Hz), 129.1, 128.2, 126.2, 124.6 (q, J=3.7 Hz), 124.3 (q, J=3.8 Hz), 124.1 (q, J=270.8 Hz), 124.0, 120.9, 120.5, 46.8; ¹⁹F NMR (376 MHz, CDCl₃, δ): −62.5.

### JMM033

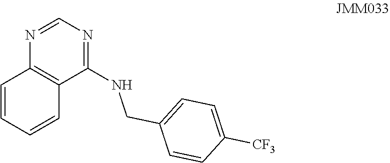 [SMILES: FC(F)(F)c1ccc(CNc2ncnc3ccccc23)cc1]

### Procedure B (64%)

¹H NMR (400 MHz, CDCl₃, δ): 8.66 (s, 1H), 7.86 (d, J=8.4 Hz, 1H), 7.81 (d, J=8.4 Hz, 1H), 7.76-7.72 (m, 1H), 7.57 (d, J=8 Hz, 1H), 7.50-7.44 (m, 3H), 6.52 (brs, 1H), 4.95 (d, J=5.6 Hz, 1H); ¹³C NMR (100 MHz, CDCl₃, δ): 159.3, 155.2, 149.4, 142.3, 132.9, 129.9 (q, J=32.3 Hz), 128.6, 128.0, 126.3, 125.7 (q, J=3.9 Hz), 124.0 (q, J=270.4 Hz), 120.4, 114.7, 44.6; ¹⁹F NMR (376 MHz, CDCl₃, δ): −62.5.

### JMM034

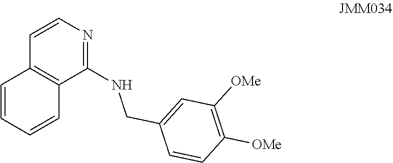 [SMILES: COc1ccc(CNc2nccc3ccccc23)cc1OC]

### Procedure B (45%)

¹H NMR (400 MHz, CDCl₃, δ): 8.04 (d, J=5.6 Hz, 1H), 7.74 (d, J=8.4 Hz, 1H), 7.69 (d, J=8 Hz, 1H), 7.59 (ddd, J=8.2, 6.8, 1.2 Hz, 1H), 7.44 (ddd, J=8.4, 6.8, 1.2 Hz, 1H), 7.0-6.97 (m, 3H), 6.86 (d, J=8.4 Hz, 1H), 5.41 (brs, 1H), 4.74 (d, J=5.2 Hz, 2H), 3.88 (s, 3H), 3.87 (s, 3H); ¹³C NMR (100 MHz, CDCl₃, δ: 154.8, 149.2, 148.5, 141.2, 137.1, 131.9, 129.8, 127.2, 126.0, 121.4, 120.4, 118.1, 111.6, 111.3, 111.2, 55.99, 55.92, 46.0;

### JMM035

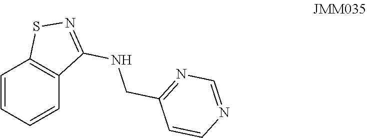 [SMILES: c1ccc2c(NCc3ccncn3)nsc2c1]

### Procedure B (19%)

¹H NMR (400 MHz, CDCl₃, δ): 9.21 (d, J=1.6 Hz, 1H), 8.68 (d, J=5.2 Hz, 1H), 7.83 (m, 2H), 7.51 (ddd, J=8.2, 6.8, 1.2 Hz, 1H), 7.42-7.38 (m, 2H), 6.20 (brs, 1H), 4.9 (s, 2H); ¹³C NMR (100 MHz, CDCl₃, δ: 166.2, 158.5, 158.3, 157.0, 151.8, 128.2, 126.2, 124.0, 121.2, 120.5, 119.4, 47.1.

### JMM036

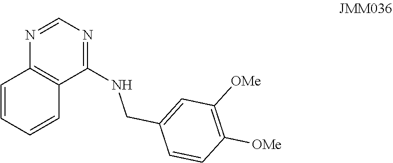 [SMILES: COc1ccc(CNc2ncnc3ccccc23)cc1OC]

### Procedure B (61%)

¹H NMR (400 MHz, CDCl₃, δ): 8.70 (s, 1H), 7.84 (d, J=8 Hz, 1H), 7.76-7.72 (m, 2H), 7.43 (ddd, J=8.2, 6.8, 1.2 Hz, 1H), 6.93 (d, J=7.6 Hz, 1H), 6.92 (s, 1H), 6.82 (d, J=8 Hz, 1H), 6.18 (brs, 1H), 4.78 (d, J=5.2 Hz, 1H), 3.86 (s, 3H), 3.83 (s, 3H); ¹³C NMR (100 MHz, CDCl₃, δ: 159.3, 155.4, 149.4, 149.2, 148.7, 132.7, 130.5, 128.5, 126.1, 120.7, 120.4, 114.9, 111.5, 111.3, 56.0, 55.9.

### JMM037

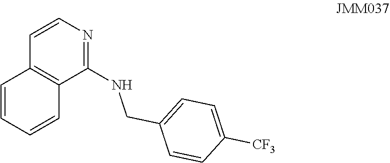 [SMILES: FC(F)(F)c1ccc(CNc2nccc3ccccc23)cc1]

### Procedure B (23%)

¹H NMR (400 MHz, CDCl₃, δ): 8.0 (d, J=6 Hz, 1H), 7.78 (d, J=8 Hz, 1H), 7.72 (d, J=8 Hz, 1H), 7.62-7.60 (m, 3H), 7.54 (d, J=8.4 Hz, 2H), 7.48 (ddd, J=8.4, 6.8, 1.2 Hz, 1H), 7.0 (d, J=6 Hz, 1H), 5.58 (brs, 1H), 4.92 (d, J=5.6 Hz, 2H); ¹³C NMR (100 MHz, CDCl₃, δ): 154.6, 143.8, 141.2, 137.1, 129.9, 129.5 (q, J=32.1 Hz), 127.9, 127.3, 126.1, 125.5 (q, J=3.8 Hz), 124.2 (q, J=270.3 Hz), 121.3, 118.0, 111.7, 45.2; ¹⁹F NMR (376 MHz, CDCl₃, δ): −62.4.

### JMM038

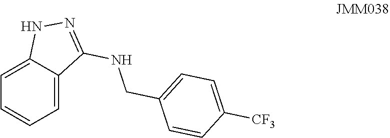 [SMILES: FC(F)(F)c1ccc(CNc2n[nH]c3ccccc23)cc1]

### Procedure G (23%)

¹H NMR (400 MHz, CDCl₃, δ): 7.6-7.54 (m, 5H), 7.36 (ddd, J=8.4, 6.8, 1.2 Hz, 1H), 7.29 (d, J=8.4 Hz, 2H), 7.06 (ddd, J=8, 6.8, 1.2 Hz, 1H), 4.71 (s, 2H); ¹³C NMR (100 MHz, CDCl₃, δ: 150.2, 143.8, 142.5, 129.5 (q, J=32.3 Hz), 127.8, 127.7, 125.5 (q, J=3.8 Hz), 124.2 (q, J=270.2 Hz), 119.3, 119.0, 114.1, 109.9, 47.6; ¹⁹F NMR (376 MHz, CDCl₃, δ): −62.4.

### JMM039

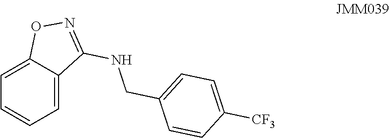 [SMILES: FC(F)(F)c1ccc(CNc2noc3ccccc23)cc1]

### Procedure G (20%)

¹H NMR (400 MHz, CDCl₃, δ): 7.60 (d, J=8.4 Hz, 2H), 7.55-7.49 (m, 4H), 7.43-7.41 (m, 1H), 7.22 (ddd, J=8, 6.8, 0.8 Hz, 1H), 4.68 (brs, 1H), 4.68 (s, 2H); ¹³C NMR (100 MHz, CDCl₃, δ: 163.0, 158.3, 142.4, 130.1, 129.9 (q, J=32.3 Hz), 128.1, 125.6 (q, J=3.7 Hz), 124.1 (q, J=270.5 Hz), 122.4, 119.6, 115.8, 110.2, 47.3; ¹⁹F NMR (376 MHz, CDCl₃, δ): −62.5.

### JMM040

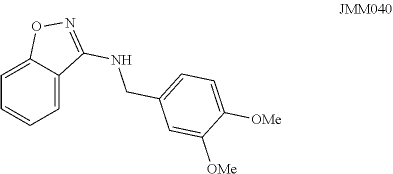 [SMILES: COc1ccc(CNc2noc3ccccc23)cc1OC]

### Procedure G (18%)

¹H NMR (400 MHz, CDCl₃, δ): 7.52-7.47 (m, 2H), 7.42 (d, J=8 Hz, 1H), 7.2 (ddd, J=7.8, 7.2, 0.8 Hz, 1H), 6.99-6.97 (m, 2H), 6.85 (d, J=8.4 Hz, 1H), 4.54 (s, 2H), 3.88 (s, 3H), 3.88 (s, 3H); ¹³C NMR (100 MHz, CDCl₃, δ: 162.9, 158.4, 149.2, 148.7, 130.9, 130.0, 122.2, 120.5, 119.8, 116.1, 111.6, 111.3, 110.1, 56.0, 55.9, 47.9.

### JMM041

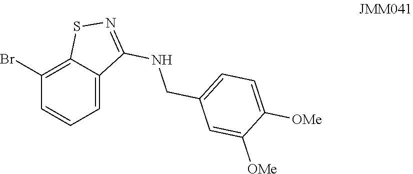 [SMILES: COc1ccc(CNc2nsc3c(Br)cccc23)cc1OC]

### Procedure G (55% Yield)

¹H NMR (400 MHz, CDCl₃, δ): 7.61 (d, J=7.6 Hz, 2H), 7.23 (dd, J=7.8, 8 Hz, 1H), 6.99-6.96 (m, 2H), 6.85 (d, J=8 Hz, 1H), 5.11 (brs, 1H), 4.69 (s, 2H), 3.88 (s, 3H), 3.87 (s, 3H); ¹³C NMR (100 MHz, CDCl₃, δ: 159.0, 153.6, 149.2, 148.6, 131.2, 130.9, 127.5, 125.5, 120.4, 120.0, 114.1, 111.6, 111.3, 56.0, 55.9, 47.2.

### JMM042

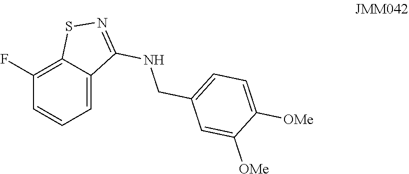 [SMILES: COc1ccc(CNc2nsc3c(F)cccc23)cc1OC]

### Procedure G (15%)

¹H NMR (400 MHz, CDCl₃, δ): 7.43 (d, J=8 Hz, 1H), 7.33-7.28 (m, 1H), 7.18-7.13 (m, 1H), 6.98-6.96 (m, 2H), 6.85 (d, J=8 Hz, 1H), 5.15 (brs, 1H), 4.69 (s, 2H), 3.88 (s, 3H), 3.87 (s, 3H); ¹³C NMR (100 MHz, CDCl₃, δ: 158.6 (d, J=1.7 Hz), 156.0 (d, J=246.4 Hz), 149.2, 148.6, 139.3 (d, J=22.3 Hz), 131.2, 130.1 (d, J=4.9 Hz), 125.9 (d, J=5.9 Hz), 120.4, 116.8 (d, J=3.8 Hz), 113.2 (d, J=18.5 Hz), 111.5, 111.3, 56.0, 55.9, 47.4; ¹⁹F NMR (376 MHz, CDCl₃, δ): −114.5.

### JMM043

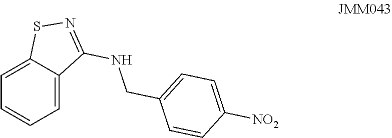 [SMILES: O=[N+]([O-])c1ccc(CNc2nsc3ccccc23)cc1]

### Procedure G (56%)

¹H NMR (400 MHz, CDCl₃, δ): 8.18 (d, J=8.8 Hz, 2H), 8.18 (d, J=8 Hz, 1H), 7.71 (d, J=8 Hz, 1H), 7.58 (d, J=8.4 Hz, 2H), 7.51 (ddd, J=8, 7.2, 1.2 Hz, 1H), 7.38 (ddd, J=8, 6.8, 0.8 Hz, 1H), 5.43 (brs, 1H), 4.9 (s, 2H); ¹³C NMR (100 MHz, CDCl₃, δ: 158.1, 151.9, 147.2, 146.9, 128.3, 128.2, 126.1, 124.1, 123.8, 120.9, 120.6, 46.4.

### JMM044

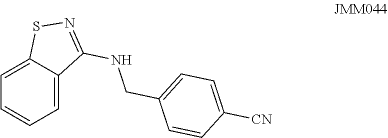 [SMILES: N#Cc1ccc(CNc2nsc3ccccc23)cc1]

### Procedure G (42%)

¹H NMR (400 MHz, CDCl₃, δ): 7.8 (d, J=8.4 Hz, 1H), 7.7 (d, J=8 Hz, 1H), 7.61 (d, J=8.4 Hz, 2H), 7.54-7.49 (m, 3H), 7.37 (ddd, J=8, 6.8, 0.8 Hz, 1H), 5.4 (brs, 1H), 4.86 (s, 2H); ¹³C NMR (100 MHz, CDCl₃, δ: 158.2, 151.9, 144.8, 132.4, 128.3, 128.2, 126.1, 124.1, 120.9, 120.6, 118.9, 111.1, 46.7.

### JMM045

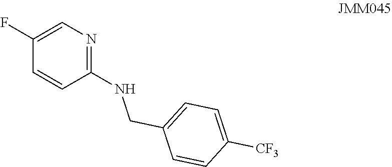 [SMILES: Fc1ccc(NCc2ccc(C(F)(F)F)cc2)nc1]

### Procedure B (37%)

¹H NMR (400 MHz, CDCl₃, δ): 7.97 (d, J=3.2 Hz, 1H), 7.58 (d, J=8.4 Hz, 1H), 7.46 (d, J=8 Hz, 1H), 7.19 (ddd, J=9.2, 8, 2.8 Hz, 1H), 6.32 (dd, J=9.2, 3.2 Hz, 1H), 4.88 (brs, 1H), 4.56 (d, J=5.2 Hz, 1H); ¹³C NMR (100 MHz, CDCl₃, δ: 154.9, 153.7 (d, J=240.8 Hz), 143.5, 134.8 (d, J=24.5 Hz), 129.5 (q, J=32.1 Hz), 127.5, 125.6 (q, J=3.8 Hz), 125.3 (d, J=20.4 Hz), 124.2 (q, J=270.7 Hz), 107.4 (d, J=3.9 Hz), 46.2; ¹⁹F NMR (376 MHz, CDCl₃, δ): −62.4, −142.9.

### JMM046

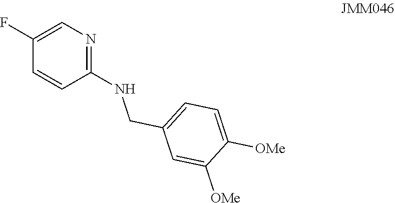 [SMILES: COc1ccc(CNc2ccc(F)cn2)cc1OC]

### Procedure G (10%)

¹H NMR (400 MHz, CDCl₃, δ): 7.96 (d, J=3.2 Hz, 1H), 7.18 (ddd, J=9, 8, 2.8 Hz, 1H), 6.90-6.81 (m, 3H), 6.33 (dd, J=8.8, 3.2 Hz, 1H), 4.80 (brs, 1H), 4.39 (d, J=5.6 Hz, 2H), 3.86 (s, 3H), 3.85 (s, 3H); ¹³C NMR (100 MHz, CDCl₃, δ: 155.4, 153.5 (d, J=240.1 Hz), 149.2, 148.3, 134.7 (d, J=24.5 Hz), 131.5, 125.2 (d, J=20.4 Hz), 119.6, 111.2, 110.7, 107.1 (d, J=3.8 Hz), 56.0, 55.9, 46.7; ¹⁹F NMR (376 MHz, CDCl₃, δ): −143.4.

### JMM047

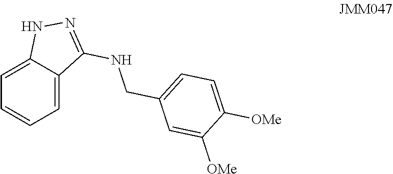 [SMILES: COc1ccc(CNc2n[nH]c3ccccc23)cc1OC]

### Procedure G (18%)

¹H NMR (400 MHz, CDCl₃, δ): 9.95 (brs, 1H), 7.72 (d, J=8.4 Hz, 1H), 7.49 (ddd, J=8.8, 6.8, 0.8 Hz, 1H), 7.35 (d, J=8.8 Hz, 1H), 7.08 (ddd, J=8.4, 6.8, 0.8 Hz, 1H), 6.95-6.92 (m, 2H), 6.86 (d, J=8 Hz, 1H), 5.53 (t, J=6 Hz, 1H), 4.85 (d, J=6 Hz, 1H), 3.87 (s, 3H), 3.87 (s, 3H); ¹³C NMR (100 MHz, CDCl₃, δ: 149.6, 148.8, 147.8, 141.8, 131.3, 130.0, 122.6, 121.4, 119.2, 112.0, 111.5, 110.8, 110.0, 56.0, 55.97, 48.4.

### JMM048

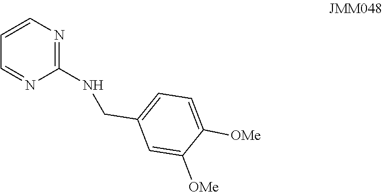 [SMILES: COc1ccc(CNc2ncccn2)cc1OC]

### Procedure G (6%)

¹H NMR (400 MHz, CDCl₃, δ): 8.28 (d, J=4.8 Hz, 2H), 6.91-6.81 (m, 3H), 6.55 (t, J=4.8 Hz, 1H), 5.81 (brs, 1H), 4.56 (d, J=6 Hz, 2H), 3.86 (s, 3H), 3.85 (s, 3H); ¹³C NMR (100 MHz, CDCl₃, δ: 163.2, 162.0, 158.0, 149.1, 148.3, 131.5, 119.7, 111.2, 110.9, 110.8, 56.0, 55.9, 45.3;

### JMM049

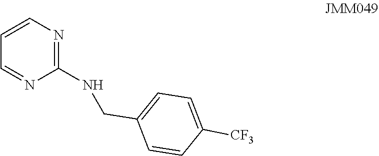 [SMILES: FC(F)(F)c1ccc(CNc2ncccn2)cc1]

### Procedure G (11%)

¹H NMR (400 MHz, CDCl₃, δ): 8.27 (d, J=4.8 Hz, 2H), 7.58 (d, J=8 Hz, 2H), 7.46 (d, J=8 Hz, 2H), 6.57 (t, J=4.8 Hz, 1H), 5.96 (brs, 1H), 4.71 (d, J=6 Hz, 2H); ¹³C NMR (100 MHz, CDCl₃, δ: 162.1, 158.1, 143.4, 129.4 (q, J=32.1 Hz), 127.4, 125.5 (q, J=3.8 Hz), 124.2 (q, J=270.4 Hz), 111.2, 44.8; ¹⁹F NMR (376 MHz, CDCl₃, δ): −62.4.

### JMM050

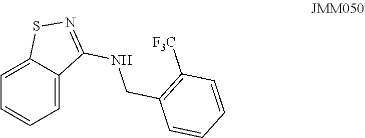 [SMILES: FC(F)(F)c1ccccc1CNc1nsc2ccccc12]

### Procedure B (76%)

¹H NMR (400 MHz, CDCl₃, δ): 7.80 (d, J=8.4 Hz, 1H), 7.73 (d, J=8 Hz, 1H), 7.69 (d, J=8 Hz, 1H), 7.75 (d, J=8 Hz, 1H), 7.53-7.47 (m, 2H), 7.41-7.33 (m, 2H), 5.26 (brs, 1H), 5.0 (d, J=6 Hz, 2H); ¹³C NMR (100 MHz, CDCl₃, δ: 158.3, 151.9, 137.5, 134.2 (q, J=50.5 Hz), 132.2, 130.5, 128.1, 127.5, 126.2, 126.1 (q, J=5.7 Hz), 124.6 (q, J=272.2 Hz), 123.9, 120.9, 120.5, 43.6 (q, J=2.5 Hz); ¹⁹F NMR (376 MHz, CDCl₃, δ): −59.4.

### JMM051

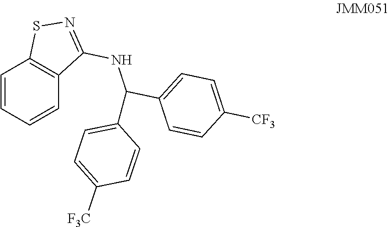 [SMILES: FC(F)(F)c1ccc(C(Nc2nsc3ccccc23)c2ccc(C(F)(F)F)cc2)cc1]

### Procedure F (10%)

¹H NMR (400 MHz, CDCl₃, δ): 7.81 (d, J=8 Hz, 1H), 7.73 (d, J=8 Hz, 1H), 7.62 (d, J=8 Hz, 4H), 7.54-7.49 (m, 5H), 7.38 (ddd, J=8.4, 6.8, 0.8 Hz, 1H), 6.51 (d, J=5.6 Hz, 1H), 5.39 (d, J=6 Hz, 1H); ¹³C NMR (100 MHz, CDCl₃, δ: 157.1, 151.9, 145.6, 130.1 (q, J=32.4 Hz), 128.3, 127.9, 126.1, 125.9 (q, J=3.7 Hz), 124.1, 124.0 (q, J=270.4 Hz), 120.9, 120.6, 60.3; ¹⁹F NMR (376 MHz, CDCl₃, δ): −62.5.

### JMM052

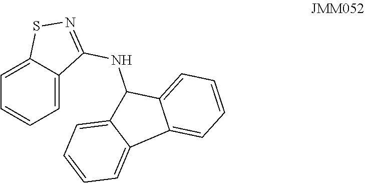 [SMILES: c1ccc2c(c1)-c1ccccc1C2Nc1nsc2ccccc12]

### Procedure H (13%)

¹H NMR (400 MHz, CDCl₃, δ): 7.85 (d, J=8 Hz, 1H), 7.75-7.72 (m, 4H), 7.55 (d, J=8 Hz, 1H), 7.51 (ddd, J=8.4, 6.8, 0.8 Hz, 1H), 7.44-7.40 (m, 2H), 7.34-7.27 (m, 3H), 6.96 (d, J=9.2 Hz, 1H), 5.19 (d, J=9.2 Hz, 1H); ¹³C NMR (100 MHz, CDCl₃, δ: 158.8, 152.0, 145.4, 140.6, 128.7, 128.2, 127.8, 126.5, 125.5, 124.0, 121.1, 120.5, 119.9, 58.0.

### JMM053

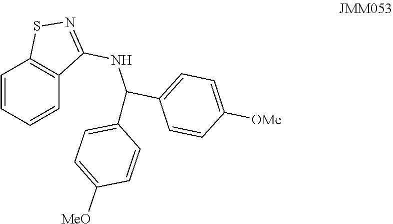 [SMILES: COc1ccc(C(Nc2nsc3ccccc23)c2ccc(OC)cc2)cc1]

### Procedure F (42%)

¹H NMR (400 MHz, CDCl₃, δ): 7.77 (d, J=8 Hz, 1H), 7.69 (d, J=8 Hz, 1H), 7.48 (ddd, J=8, 7.2, 1.2 Hz, 1H), 7.34 (ddd, J=8, 6.8, 1.2 Hz, 1H), 7.28 (d, J=8.4 Hz, 4H), 6.86 (d, J=8.8 Hz, 4H), 6.31 (d, J=6 Hz, 4H), 5.35 (d, J=6 Hz, 4H), 3.79 (s, 6H); ¹³C NMR (100 MHz, CDCl₃, δ: 158.8, 157.6, 151.7, 135.0, 128.6, 128.0, 126.4, 123.8, 120.9, 120.4, 114.0, 59.6, 55.3.

### JMM054

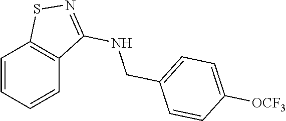 [SMILES: FC(F)(F)Oc1ccc(CNc2nsc3ccccc23)cc1]

### Procedure B (11%)

¹H NMR (400 MHz, CDCl₃, δ): 7.80 (d, J=8.4 Hz, 1H), 7.66 (d, J=8 Hz, 1H), 7.50 (ddd, J=8.4, 7.2, 1.2 Hz, 1H), 7.46 (d, J=8.8 Hz, 2H), 7.35 (ddd, J=8.4, 6.8, 0.8 Hz, 1H), 7.20 (d, J=8.4 Hz, 1H), 5.21 (brs, 1H), 4.80 (d, J=5.2 Hz, 1H); ¹³C NMR (100 MHz, CDCl₃, δ: 158.5, 151.9, 148.5, 137.9, 129.3, 128.2, 124.0, 121.2, 120.9, 120.53, 120.49 (q, J=255.4 Hz), 114.7, 46.5; ¹⁹F NMR (376 MHz, CDCl₃, δ): −57.8.

### JMM055

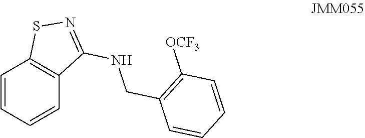 [SMILES: FC(F)(F)Oc1ccccc1CNc1nsc2ccccc12]

### Procedure B (13%)

¹H NMR (400 MHz, CDCl₃, δ): 10.55 (d, J=0.8 Hz, 1H), 7.87 (dd, J=7.6, 1.6 Hz, 1H), 7.62 (dd, J=5.2, 2 Hz, 1H), 7.56 (ddd, J=8.4, 7.2, 1.6 Hz, 1H), 7.43-7.31 (m, 3H), 7.10-7.04 (m, 2H), 5.28 (s, 2H); ¹³C NMR (100 MHz, CDCl₃, δ: 189.5, 160.6, 136.0, 129.6, 129.3, 128.9, 128.7, 127.2, 125.3, 121.3, 120.67, 120.66, 120.6 (q, J=256.5 Hz), 112.8, 64.9; ¹⁹F NMR (376 MHz, CDCl₃, δ): −57.2.

### JMM056

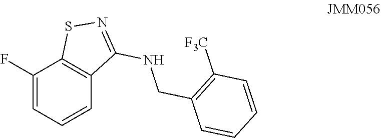 [SMILES: Fc1cccc2c(NCc3ccccc3C(F)(F)F)nsc12]

### Procedure G (32%)

¹H NMR (400 MHz, CDCl₃, δ): 7.72 (d, J=7.6 Hz, 1H), 7.69 (d, J=8 Hz, 1H), 7.54-7.50 (m, 1H), 7.44-7.38 (m, 2H), 7.33 (ddd, J=8, 8, 4.4 Hz, 1H), 7.16 (ddd, J=8, 8, 4.4 Hz, 1H), 7.16 (J=9.4, 8, 0.8 Hz, 1H), 5.26 (brs, 1H), 4.99 (d, J=6.4 Hz, 2H); ¹³C NMR (100 MHz, CDCl₃, δ: 158.2 (d, J=1.7 Hz), 156.0 (d, J=246.4 Hz), 139.5 (d, J=22.3 Hz), 137.2, 132.2, 130.6, 129.9 (d, J=5 Hz), 128.3 (q, J=30.3 Hz), 127.6, 126.1 (q, J=5.7 Hz), 126.0 (d, J=6 Hz), 124.6 (q, J=271.8 Hz), 116.6 (d, J=3.8 Hz), 113.3 (d, J=18.5 Hz), 43.8 (q, J=2.5 Hz); ¹⁹F NMR (376 MHz, CDCl₃, δ): −59.4, −114.5.

### JMM057

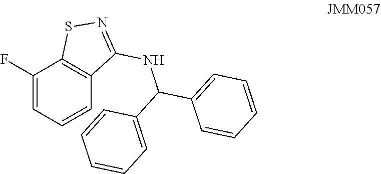 [SMILES: Fc1cccc2c(NC(c3ccccc3)c3ccccc3)nsc12]

### Procedure H (6%)

¹H NMR (400 MHz, CDCl₃, δ): 7.50 (d, J=8 Hz, 1H), 7.39-7.26 (m, 11H), 7.16 (ddd, J=9.4, 7.6, 0.8 Hz, 1H), 6.39 (d, J=6 Hz, 1H), 5.44 (d, J=6 Hz, 1H); ¹³C NMR (100 MHz, CDCl₃, δ: 157.4 (d, J=1.5 Hz), 156.0 (d, J=246.2 Hz), 142.3, 139.4 (d, J=22.2 Hz), 130.1 (d, J=5.2 Hz), 128.7, 127.5, 127.47, 125.9 (d, J=6 Hz), 116.7 (d, J=3.8 Hz), 113.2 (d, J=18.6 Hz), 60.9; ¹⁹F NMR (376 MHz, CDCl₃, δ): −114.4.

### JMM058

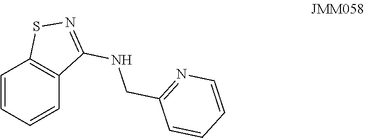 [SMILES: c1ccc(CNc2nsc3ccccc23)nc1]

### Procedure B (32%)

¹H NMR (400 MHz, CDCl₃, δ): 8.60 (d, J=4.8 Hz, 1H), 7.84 (d, J=8 Hz, 1H), 7.78 (d, J=8 Hz, 1H), 7.68 (ddd, J=7.6, 7.6, 1.6 Hz), 7.49 (d, J=8, 7.2, 1.2 Hz, 1H), 7.39-7.35 (m, 2H), 7.24-7.20 (m, 1H), 6.41 (brs, 1H), 4.90 (d, J=4.8 Hz, 2H); ¹³C NMR (100 MHz, CDCl₃, δ: 158.9, 157.1, 151.6, 149.0, 136.7, 128.1, 126.6, 123.9, 122.3, 122.2, 121.4, 120.4, 47.6.

### JMM059

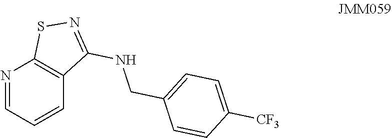 [SMILES: FC(F)(F)c1ccc(CNc2nsc3ncccc23)cc1]

### Procedure B (64%)

¹H NMR (400 MHz, CDCl₃, δ): 8.71 (d, J=4.8 Hz, 1H), 7.98 (d, J=9.6, 1.6 Hz, 1H), 7.59 (d, J=8 Hz, 2H), 7.52 (d, J=8 Hz, 2H), 7.28 (dd, J=8.4, 4.8 Hz, 1H), 5.41 (brs, 1H), 4.82 (d, J=5.6 Hz, 1H); ¹³C NMR (100 MHz, CDCl₃, δ: 172.4, 156.4, 150.6, 142.8, 129.8 (q, J=32.2 Hz), 129.3, 128.1, 125.6 (q, J=3.8 Hz), 124.1 (q, J=270.3 Hz), 119.4, 118.6, 46.2; 19F NMR (376 MHz, CDCl₃, δ): −62.5.

### JMM060

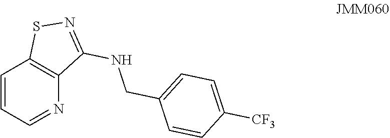 [SMILES: FC(F)(F)c1ccc(CNc2nsc3cccnc23)cc1]

### Procedure B (24%)

¹H NMR (400 MHz, CDCl₃, δ): 8.6 (dd, J=4.8, 1.2 Hz, 1H), 8.14 (dd, J=8.4, 1.2 Hz, 1H), 7.60 (d, J=8 Hz, 2H), 7.54 (d, J=7.6 Hz, 2H), 7.41 (dd, J=8, 4.4 Hz, 1H), 6.26 (brs, 1H), 4.87 (d, J=6 Hz, 2H); ¹³C NMR (100 MHz, CDCl₃, δ: 158.3, 146.7, 144.5, 143.0, 141.8, 129.6 (q, J=32.4 Hz), 128.7, 127.8, 125.6 (q, J=3.8 Hz), 124.2 (q, J=269.9 Hz), 122.4, 46.1; ¹⁹F NMR (376 MHz, CDCl₃, δ): −62.4.

### JMM061

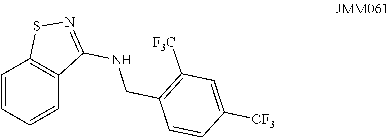 [SMILES: FC(F)(F)c1ccc(CNc2nsc3ccccc23)c(C(F)(F)F)c1]

### Procedure B (24%)

¹H NMR (400 MHz, CDCl₃, δ): 7.93 (s, 1H), 7.89 (d, J=8 Hz, 1H), 7.81 (d, J=8.4 Hz, 1H), 7.74 (d, J=8.4 Hz, 1H), 7.68 (d, J=8.4 Hz, 1H), 7.51 (ddd, J=8.4, 6.8, 0.8 Hz, 1H), 7.38 (ddd, J=8, 7.2, 0.8 Hz, 1H), 5.39 (brs, 1H), 5.06 (d, J=5.6 Hz, 1H); ¹³C NMR (100 MHz, CDCl₃, δ: 157.9, 152.0, 142.0, 130.7, 129.9 (q, J=33.3 Hz), 128.9 (q, J=3.3 Hz), 128.8 (q, J=31.3 Hz), 128.3, 126.0, 124.1, 123.3 (q, J=3.8 Hz), 123.8 (q, J=272.6 Hz), 123.4 (q, J=270.5 Hz), 120.8, 120.6, 43.3 (q, J=2.7 Hz); ¹⁹F NMR (376 MHz, CDCl₃, δ): −60.2, −62.8.

### JMM062

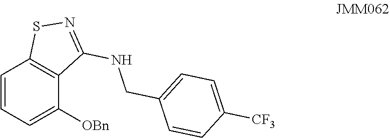 [SMILES: FC(F)(F)c1ccc(CNc2nsc3cccc(OCc4ccccc4)c23)cc1]

### Procedure G (22%)

¹H NMR (400 MHz, CDCl₃, δ): 7.51 (d, J=8 Hz, 2H), 7.39-7.26 (m, 9H), 6.77 (d, J=7.2 Hz, 1H), 6.64 (t, J=5.2 Hz, 1H), 5.16 (s, 2H), 4.70 (d, J=5.2 Hz, 2H); ¹³C NMR (100 MHz, CDCl₃, δ: 159.0, 155.9, 154.6, 143.1, 135.4, 129.5, 129.0 (q, J=32.1 Hz), 128.8, 128.7, 127.81, 127.76, 125.5 (q, J=3.7 Hz), 124.2 (q, J=270.3 Hz), 116.6, 113.1, 105.1, 70.8, 46.5; ¹⁹F NMR (376 MHz, CDCl₃, δ): −62.4.

### JMM063

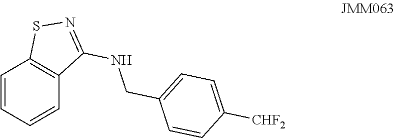 [SMILES: FC(F)c1ccc(CNc2nsc3ccccc23)cc1]

### Procedure B (28%)

¹H NMR (400 MHz, CDCl₃, δ): 7.80 (d, J=8 Hz, 1H), 7.66 (d, J=8.4 Hz, 1H), 7.54-7.48 (m, 5H), 7.36 (ddd, J=8, 7.2, 1.2 Hz, 1H), 6.64 (t, J=56.4 Hz, 1H), 5.24 (s, 1H), 4.84 (d, J=5.6 Hz, 2H); ¹³C NMR (100 MHz, CDCl₃, δ: 158.5, 151.8, 142.0, 133.6 (t, J=22.4 Hz), 128.2, 128.1, 126.2, 125.9 (t, J=6.1 Hz), 124.0, 120.9, 120.5, 114.6 (t, J=237.2 Hz), 46.8; ¹⁹F NMR (376 MHz, CDCl₃, δ): −110.3.

### JMM064

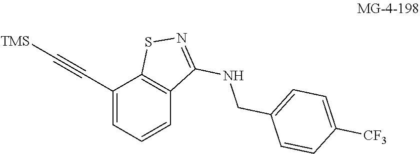 [SMILES: C[Si](C)(C)C#Cc1cccc2c(NCc3ccc(C(F)(F)F)cc3)nsc12]

In a argon-filled flask, [Pd(PPh₃)₄] (0.02 mmol, 10 mol %), CuI (0.02 mmol, 10 mol %) and JMM028 (0.2 mmol) were added, followed by adding dry THE (5 mL) and triethylamine (5 mL). Then trimethylsilylacetylene (42 μL, 1.5 equiv.) was added to the reaction mixture slowly. The reaction mixture was stirred at 100° C. for 24 h. Reaction mixture was cooled to room temperature and passed through a short pad of celite using ethyl acetate. Solvent was evaporated under reduced pressure. The crude product was purified by silicon gel column chromatography (EtOAc/Hexane). (62%)[7]

¹H NMR (400 MHz, CDCl₃, δ): 7.62-7.52 (m, 6H), 7.33 (d, J=7.2 Hz, 1H), 7.31 (d, J=7.2 Hz, 1H), 5.21 (t, J=5.6 Hz, 1H), 4.84 (d, J=5.6 Hz, 2H), 0.30 (s, 9H); ¹³C NMR (100 MHz, CDCl₃, δ: 158.6, 155.0, 143.1, 131.4, 129.7 (q, J=32.3 Hz), 128.0, 126.2, 125.6 (q, J=3.7 Hz), 124.3, 124.1 (q, J=270.3 Hz), 120.9, 116.2, 101.2, 101.0, 46.7, −0.1; ¹⁹F NMR (376 MHz, CDCl₃, δ): −62.4.

### JMM064

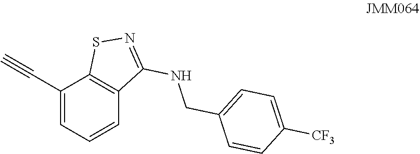 [SMILES: C#Cc1cccc2c(NCc3ccc(C(F)(F)F)cc3)nsc12]

The TMS-alkyne (0.12 mmol) is dissolved in MeOH (5 mL), K₂CO₃ (0.24 mmol) is added and the mixture is stirred at room temperature overnight. The solvent is removed in vacuo and purified by column chromatography on silicon gel using EtOAc/Hexane. (51%)[8]

¹H NMR (400 MHz, CDCl₃, δ): 7.66-7.59 (m, 4H), 7.53 (d, J=7.6 Hz, 2H), 7.34 (dd, J=8, 7.2 Hz, 1H), 5.28 (t, J=5.6 Hz, 1H), 4.84 (d, J=5.6 Hz, 2H), 3.47 (s, 1H); ¹³C NMR (100 MHz, CDCl₃, δ: 158.6, 154.9, 143.0, 132.0, 129.7 (q, J=32.1 Hz), 128.0, 126.3, 125.6 (q, J=3.8 Hz), 124.3, 124.1 (q, J=270.2 Hz), 121.4, 115.0, 83.1, 80.1, 46.6; ¹⁹F NMR (376 MHz, CDCl₃, δ): −62.4.

### JMM065

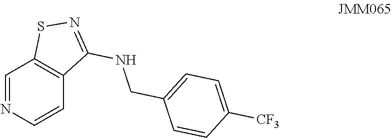 [SMILES: FC(F)(F)c1ccc(CNc2nsc3cnccc23)cc1]

### Procedure B (22%)

¹H NMR (400 MHz, CDCl₃, δ): 9.16 (s, 1H), 8.52 (d, J=5.6 Hz, 1H), 7.61-7.56 (m, 3H), 7.52 (d, J=8 Hz, 2H), 5.57 (t, J=5.6 Hz, 1H), 4.84 (d, J=5.6 Hz, 1H); ¹³C NMR (100 MHz, CDCl₃, δ: 158.0, 147.8, 143.4, 142.8, 142.6, 131.1, 129.9 (q, J=32.2 Hz), 128.0, 125.7 (q, J=3.9 Hz), 124.1 (q, J=270.3 Hz), 114.9, 46.8; ¹⁹F NMR (376 MHz, CDCl₃, δ): −62.5.

### JMM066

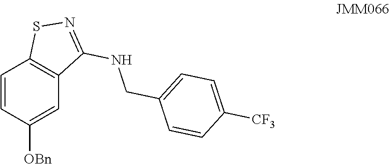 [SMILES: FC(F)(F)c1ccc(CNc2nsc3ccc(OCc4ccccc4)cc23)cc1]

### Procedure G (6%)

¹H NMR (400 MHz, CDCl₃, δ): 7.68 (d, J=8.8 Hz, 1H), 7.61 (d, J=8.4 Hz, 2H), 7.54 (d, J=8 Hz, 2H), 7.46-7.35 (m, 5H), 7.24 (d, J=8.8, 2.4 Hz, 1H), 7.13 (d, J=2.4 Hz, 1H), 5.12 (s, 2H), 4.84 (s, 2H); ¹³C NMR (100 MHz, CDCl₃, δ: 157.9, 126.6, 144.8, 143.2, 136.4, 128.7, 128.4 (q, J=17 Hz), 128.2, 128.0, 127.5, 127.0, 125.6 (q, J=3.8 Hz), 124.2 (q, J=269.8 Hz), 121.3, 119.5, 104.0, 70.7, 46.8; ¹⁹F NMR (376 MHz, CDCl₃, δ): −62.4.

### JMM067

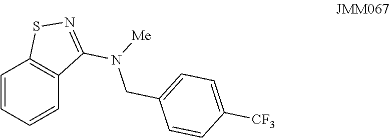 [SMILES: CN(Cc1ccc(C(F)(F)F)cc1)c1nsc2ccccc12]

### Procedure I (79%)

¹H NMR (400 MHz, CDCl₃, δ): 7.87 (d, J=8.4 Hz, 1H), 7.82 (d, J=8.4 Hz, 1H), 7.65 (d, J=8 Hz, 1H), 7.55 (d, J=8.4 Hz, 1H), 7.46 (ddd, J=8.4, 6.8, 0.8 Hz), 7.30 (ddd, J=8.4, 7.2, 1.2 Hz), 4.81 (s, 2H), 3.16 (s, 2H); ¹³C NMR (100 MHz, CDCl₃, δ: 163.3, 153.2, 142.6, 129.7 (q, J=32.1 Hz), 127.7, 127.54, 127.49, 125.7 (q, J=3.8 Hz), 124.2 (q, J=270.3 Hz), 124.0, 123.9, 120.7, 57.2, 39.2; ¹⁹F NMR (376 MHz, CDCl₃, δ): −62.4.

### JMM068

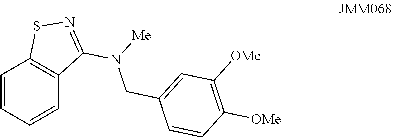 [SMILES: COc1ccc(CN(C)c2nsc3ccccc23)cc1OC]

### Procedure I (37%)

¹H NMR (400 MHz, CDCl₃, δ): 7.91 (d, J=8 Hz, 1H), 7.80 (d, J=8 Hz, 1H), 7.45-7.42 (m, 1H), 7.3-7.28 (m, 1H), 6.97-6.94 (m, 2H), 6.88 (d, J=8 Hz, 1H), 4.69 (s, 2H), 3.89 (s, 3H), 3.85 (s, 3H), 3.12 (s, 3H); ¹³C NMR (125 MHz, CDCl₃, δ): 163.7, 153.0, 149.3, 148.3, 130.7, 127.7, 127.4, 124.1, 123.9, 120.6, 119.5, 111.2, 110.4, 57.3, 56.0, 55.9, 38.7.

### JMM069

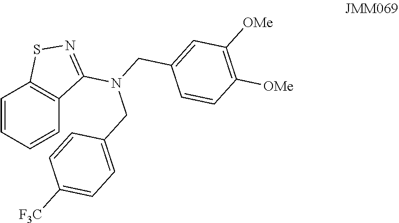 [SMILES: COc1ccc(CN(Cc2ccc(C(F)(F)F)cc2)c2nsc3ccccc23)cc1OC]

### Procedure I (19%)

¹H NMR (400 MHz, CDCl₃, δ): 7.90 (d, J=8.4 Hz, 1H), 7.83 (d, J=8.4 Hz, 1H), 7.57 (d, J=8.4 Hz, 2H), 7.49-7.45 (m, 3H), 7.29 (ddd, J=8.4, 6.8, 0.8 Hz, 1H), 6.88-6.82 (m, 3H), 4.78 (s, 2H), 4.73 (s, 2H), 3.88 (s, 3H), 3.79 (s, 3H); ¹³C NMR (100 MHz, CDCl₃, δ: 162.4, 153.4, 149.2, 148.4, 142.4, 129.9, 129.4 (q, J=32.2 Hz), 128.2, 127.6, 127.4, 125.4 (q, J=3.8 Hz), 124.2 (q, J=270.2 Hz), 124.1, 123.9, 120.7, 120.1, 111.2, 111.0, 55.9, 55.8, 54.3, 53.3; ¹⁹F NMR (376 MHz, CDCl₃, δ): −62.4.

### JMM070

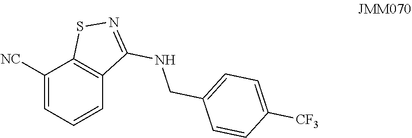 [SMILES: N#Cc1cccc2c(NCc3ccc(C(F)(F)F)cc3)nsc12]

A flask with JMM028 (0.154 mmol), Zn(CN)₂ (0.23 mmol) and Pd(PPh₃)₄ (10 mmol %) was added degassed DMF (10 mL) under vacuum. The reaction mixture was heated to 120° C. overnight. Water was added to reaction mixture and stirred for 5 minutes. The solvent was concentrated in vacuo and extracted by EtOAc. The organic phase was dried over Na₂SO₄ and evaporated. The residue was purified by column chromatography over silicon gel. (76%)[9]

¹H NMR (400 MHz, CDCl₃, δ): 7.89 (dd, J=8, 0.8 Hz, 1H), 7.83 (dd, J=7.2, 0.8 Hz, 1H), 7.62 (d, J=8 Hz, 2H), 7.54 (d, J=8 Hz, 2H), 7.46 (dd, J=8, 7.2 Hz, 1H), 5.34 (brs, 1H), 4.85 (d, J=6 Hz, 2H); ¹³C NMR (100 MHz, CDCl₃, δ: 158.3, 154.5, 142.6, 133.2, 129.9 (q, J=32 Hz), 128.1, 127.2, 125.7 (q, J=3.8 Hz), 125.4, 124.3, 124.1 (q, J=270.2 Hz), 116.3, 105.0, 46.8; ¹⁹F NMR (376 MHz, CDCl₃, δ): −62.5.

### JMM071

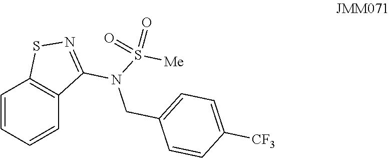 [SMILES: CS(=O)(=O)N(Cc1ccc(C(F)(F)F)cc1)c1nsc2ccccc12]

To a mixture of JMM005 (0.1 mmol, 31 mg) and pyridine (30 μL), methylsulfonyl chloride (0.12 mmol, 9.3 μL) in dry DCM was added slowly and stirred at room temperature for 3 days. The reaction mixture was then washed with aqueous 1M HCl and extracted with EtOAc. The combined organic phases were dried over anhydrous sodium sulfate. The filtered solution was concentrated and was purified by column chromatography. (13%)

¹H NMR (400 MHz, CDCl₃, δ): 9.13 (d, J=8.4 Hz, 1H), 7.69-7.64 (m, 3H), 7.57-7.51 (m, 2H), 7.47 (d, J=8 Hz, 2H), 5.19 (s, 2H), 3.19 (s, 3H); ¹³C NMR (100 MHz, CDCl₃, δ: 158.3, 141.1, 138.6, 132.5, 131.0 (q, J=32.7 Hz), 130.97, 128.9, 126.6, 126.0 (q, J=3.7 Hz), 123.8 (q, J=270.7 Hz), 123.2, 119.6, 50.0, 43.9; ¹⁹F NMR (376 MHz, CDCl₃, δ): −62.7.

### JMM072

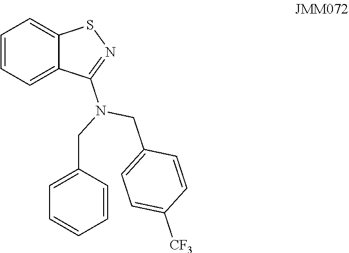 [SMILES: FC(F)(F)c1ccc(CN(Cc2ccccc2)c2nsc3ccccc23)cc1]

### Procedure I (79%)

¹H NMR (400 MHz, CDCl₃, δ): 7.90 (d, J=8 Hz, 1H), 7.84 (d, J=8 Hz, 1H), 7.58 (d, J=8.4 Hz, 2H), 7.49-7.45 (m, 3H), 7.39-7.37 (m, 4H), 7.33-7.26 (m, 2H), 4.81 (s, 4H); ¹³C NMR (100 MHz, CDCl₃, δ: 162.3, 153.4, 142.4, 137.6, 129.4 (q, J=32.1 Hz), 128.8, 128.2, 127.8, 127.6, 127.5, 127.4, 125.5 (q, J=3.8 Hz), 124.3 (q, J=270.3 Hz), 124.1, 123.9, 120.7, 54.6, 53.5; ¹⁹F NMR (376 MHz, CDCl₃, δ): −62.3.

### JMM073

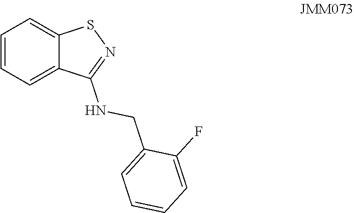 [SMILES: Fc1ccccc1CNc1nsc2ccccc12]

### Procedure B (30%)

¹H NMR (400 MHz, CDCl₃, δ): 10.54 (d, J=0.8 Hz, 1H), 7.86 (dd, J=7.6, 1.6 Hz, 1H), 7.56 (ddd, J=8.4, 7.2, 2 Hz, 1H), 7.74-7.49 (m, 1H), 7.38-7.32 (m, 1H), 7.19 (td, J=7.6, 1.2 Hz, 1H), 7.14-7.04 (m, 3H), 5.26 (s, 2H); ¹³C NMR (100 MHz, CDCl₃, δ: 189.7, 160.8, 160.4 (d, J=245.8 Hz), 135.9, 130.1 (d, J=8.2 Hz), 129.6 (d, J=3.7 Hz), 128.6, 125.3, 124.4 (d, J=3.6 Hz), 123.3 (d, J=14.1 Hz), 121.3, 115.5 (d, J=20.9 Hz), 113.0, 64.4 (d, J=4.5 Hz); ¹⁹F NMR (376 MHz, CDCl₃, δ): −118.5.

### JMM074

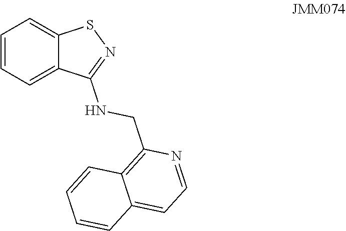 [SMILES: c1ccc2c(CNc3nsc4ccccc34)nccc2c1]

### Procedure B (56%)

¹H NMR (400 MHz, CDCl₃, δ): 8.52 (d, J=6 Hz, 1H), 8.24 (d, J=8.4 Hz, 1H), 7.99 (d, J=8 Hz, 1H), 7.87 (d, J=7.6 Hz, 1H), 7.81 (d, J=8 Hz, 1H), 7.73 (ddd, J=8.4, 6.8, 1.2 Hz, 1H), 7.69-7.62 (m, 2H), 7.51 (ddd, J=8.2, 7.2, 1.2 Hz, 1H), 7.32 (brs, 1H), 5.39 (d, J=3.6 Hz, 1H); ¹³C NMR (100 MHz, CDCl₃, δ: 159.1, 155.1, 151.5, 141.0, 136.0, 130.4, 128.1, 127.7, 127.4, 126.7, 126.0, 123.91, 123.89, 121.7, 120.38, 120.35, 44.8.

### JMM075

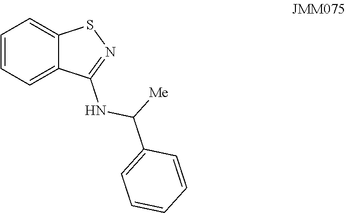 [SMILES: CC(Nc1nsc2ccccc12)c1ccccc1]

### Procedure H (5%)

¹H NMR (400 MHz, CDCl₃, δ): 7.77 (d, J=8 Hz, 1H), 7.67 (d, J=8 Hz, 1H), 7.50-7.45 (m, 3H), 7.38-7.32 (m, 3H), 7.29-7.26 (m, 1H), 5.36-5.29 (m, 1H), 5.15-5.12 (m, 1H), 1.68 (d, J=6.8 Hz, 3H); ¹³C NMR (100 MHz, CDCl₃, δ: 157.8, 151.6, 144.3, 128.7, 128.0, 127.3, 126.5, 126.2, 123.8, 120.9, 120.4, 52.3, 22.8.

### JMM076

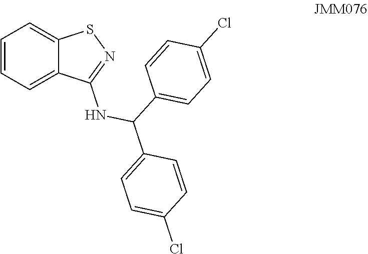 [SMILES: Clc1ccc(C(Nc2nsc3ccccc23)c2ccc(Cl)cc2)cc1]

### Procedure H (25%)

¹H NMR (400 MHz, CDCl₃, δ): 7.79 (d, J=8.4 Hz, 1H), 7.70 (d, J=8 Hz, 1H), 7.50 (ddd, J=8, 7.2, 1.2 Hz, 1H), 7.38-7.27 (m, 9H), 6.34 (d, J=5.6 Hz, 1H), 5.35 (d, J=5.6 Hz, 1H); ¹³C NMR (100 MHz, CDCl₃, δ: 157.2, 151.8, 140.6, 133.5, 129.0, 128.9, 128.2, 126.2, 124.0, 120.9, 120.5, 59.7.

### JMM077

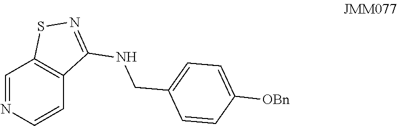 [SMILES: c1ccc(COc2ccc(CNc3nsc4cnccc34)cc2)cc1]

### Procedure B (35%)

¹H NMR (400 MHz, CDCl₃, δ): 9.16 (s, 1H), 8.52 (d, J=4.8 Hz, 1H), 7.51 (d, J=5.2 Hz, 1H), 7.45-7.30 (m, 7H), 6.98 (d, J=8.8 Hz, 2H), 5.20 (t, J=5.2 Hz, 1H), 5.08 (s, 2H), 4.70 (t, J=5.2 Hz, 2H); ¹³C NMR (100 MHz, CDCl₃, δ: 158.4, 158.2, 143.3, 142.7, 139.3, 136.9, 131.3, 130.7, 129.5, 128.6, 128.0, 127.4, 115.1, 115.0, 70.1, 46.9;

### JMM078

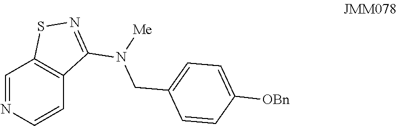 [SMILES: CN(Cc1ccc(OCc2ccccc2)cc1)c1nsc2cnccc12]

### Procedure I (86%)

¹H NMR (400 MHz, CDCl₃, δ): 9.18 (s, 1H), 8.44 (m, 1H), 7.74 (d, J=5.6 Hz, 1H), 7.45-7.33 (m, 5H), 7.29 (d, J=8.8 Hz, 2H), 6.99 (d, J=8.4 Hz, 2H), 5.07 (s, 2H), 4.74 (s, 2H), 3.18 (s, 3H); ¹³C NMR (100 MHz, CDCl₃, δ: 162.5, 158.3, 143.7, 142.6, 136.9, 132.1, 129.8, 128.6, 128.5, 128.0, 127.5, 118.0, 115.2, 115.0, 70.1, 56.5, 38.6;

### JMM079

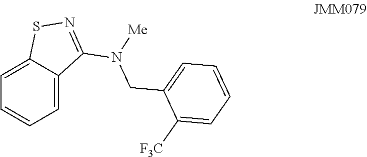 [SMILES: CN(Cc1ccccc1C(F)(F)F)c1nsc2ccccc12]

### Procedure I (82%)

¹H NMR (400 MHz, CDCl₃, δ): 7.84-7.79 (m, 2H), 7.74 (d, J=8.4, 0.8 Hz, 2H), 7.58 (t, d=7.6 Hz, 1H), 7.45-7.40 (m, 2H), 7.23 (ddd, J=8.4, 7.2, 1.2 Hz, 1H), 4.98 (s, 2H), 3.22 (s, 3H); ¹³C NMR (100 MHz, CDCl₃, δ: 163.1, 153.3, 137.2, 132.4, 128.07, 127.98 (q, J=30.5 Hz), 127.46, 127.3, 127.2, 126.3 (q, J=5.8 Hz), 124.5 (q, J=272.3 Hz), 123.92, 123.89, 120.6, 54.0 (q, J=3.1 Hz), 39.3; ¹⁹F NMR (376 MHz, CDCl₃, δ): −60.4.

### JMM-080

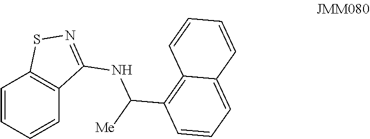 [SMILES: CC(Nc1nsc2ccccc12)c1cccc2ccccc12]

¹H NMR (400 MHz, CDCl₃, δ): 8.22-8.19 (m, 2H), 7.89-7.87 (m, 1H), 7.81 (d, J=8 Hz, 1H), 7.79 (d, J=8 Hz, 1H), 7.66 (d, J=7.2 Hz, 1H), 7.59 (d, J=8.4 Hz, 1H), 7.52-7.45 (m, 4H), 7.30 (ddd, J=8, 7.2, 0.8 Hz, 1H), 6.15-6.08 (m, 1H), 5.17 (d, J=6.4 Hz, 1H), 1.85 (d, J=6.4 Hz, 3H); ¹³C NMR (100 MHz, CDCl₃, δ: 157.7, 151.6, 139.4, 134.1, 131.3, 128.8, 128.2, 128.0, 126.4, 125.8, 125.4, 123.8, 123.6, 122.4, 121.0, 120.4, 48.3, 21.3;

### JMM081

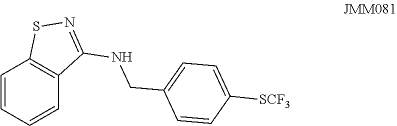

### Procedure G (26%)

¹H NMR (400 MHz, CDCl₃, δ): 7.80 (d, J=8.4 Hz, 1H), 7.67 (d, J=8.0 Hz, 1H), 7.62 (d, J=8.0 Hz, 2H), 7.52-7.46 (m, 3H), 7.35 (t, J=7.6 Hz, 2H), 5.32 (t, J=8.0, 6.0 Hz, 1H), 4.82 (d, J=5.6 Hz, 2H); ¹³C NMR (100 MHz, CDCl₃, δ): 158.4, 151.8, 142.4, 136.6, 129.6 (q, J=306.2 Hz), 128.8, 128.2, 126.2, 124.0, 123.1 (q, J=2.2 Hz), 120.9, 120.5, 46.6; ¹⁹F NMR (376 MHz, CDCl₃, δ): −42.7.

### JMM082

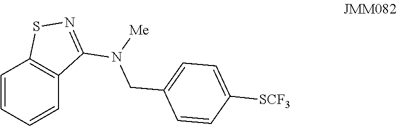

### Procedure I (89%)

¹H NMR (400 MHz, CDCl₃, δ): 7.87 (d, J=8.4 Hz, 1H), 7.81 (d, J=8.4 Hz, 1H), 7.67 (d, J=8.4 Hz, 2H), 7.50-7.44 (m, 3H), 7.30 (ddd, J=8.4, 7.2, 1.2 Hz, 1H), 4.78 (s, 2H), 3.16 (s, 3H); ¹³C NMR (100 MHz, CDCl₃, δ): 163.2, 153.2, 141.7, 136.7, 129.6 (q, J=306.2 Hz), 128.5, 127.5, 127.5, 123.97, 123.94, 123.1 (q, J=2.2 Hz), 120.6, 57.1, 39.2; ¹⁹F NMR (376 MHz, CDCl₃, δ): −42.7.

### JMM083

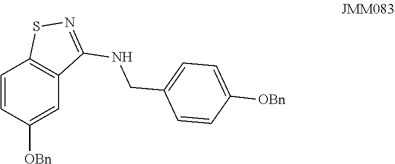 [SMILES: c1ccc(COc2ccc(CNc3nsc4ccc(OCc5ccccc5)cc34)cc2)cc1]

### Procedure G (33%)

¹H NMR (400 MHz, CD₃OD, δ): 7.66-7.63 (m, 2H), 7.44-7.24 (m, 12H), 7.20 (dd, J=8.8, 2.4 Hz, 1H), 6.92 (d, J=8.4 Hz, 2H), 5.08 (s, 2H), 5.03 (s, 2H), 4.58 (s, 2H); ¹³C NMR (100 MHz, CD₃OD, δ): 159.3, 158.0, 156.7, 143.5, 137.4, 136.9, 131.8, 128.6, 128.1, 128.0, 127.6, 127.37, 127.36, 127.31, 127.1, 120.5, 119.4, 114.5, 104.8, 70.1, 69.6, 45.7.

### JMM084

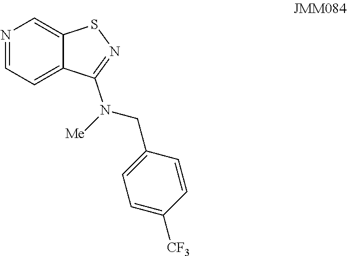 [SMILES: CN(Cc1ccc(C(F)(F)F)cc1)c1nsc2cnccc12]

### Procedure I (78%)

¹H NMR (400 MHz, CDCl₃, δ): 9.20 (s, 1H), 8.45 (d, J=5.6 Hz, 1H), 7.71 (dd, J=4.8, 1.2 Hz, 1H), 7.65 (d, J=8.0 Hz, 2H), 7.51 (d, J=7.6 Hz, 2H), 4.86 (s, 2H), 3.24 (s, 3H); ¹³C NMR (100 MHz, CDCl₃, δ): 162.1, 143.8, 142.7, 141.9, 131.9, 129.9 (q, J=32.2 Hz), 127.5, 125.8 (q, J=3.8 Hz), 124.1 (q, J=270.4 Hz), 117.7, 115.0, 56.7, 39.0; ¹⁹F NMR (376 MHz, CDCl₃, δ): −62.4.

### JMM085

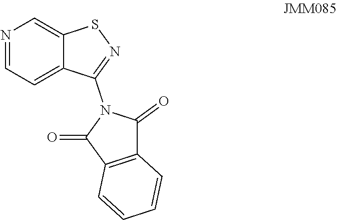 [SMILES: O=C1c2ccccc2C(=O)N1c1nsc2cnccc12]

To a solution of 3-amino benzoisothiazole (0.4 mmol, 60 mg) in acetic acid (1.5 mL) was added phthalic anhydride (0.4 mmol, 59 mg). The resulting solution was stirred and heated to 120° C. overnight. The solution was cooled to room temperature and treated with Sat. NaHCO₃. The resulting mixture was extracted with EtOAc. The organic layer was dried over Na₂SO₄ and evaporated to give the crude product. Purified by column chromatography EtOAc/Hexane. (19%)[10]

¹H NMR (400 MHz, CDCl₃, δ): 8.03 (dd, J=5.6, 3.2 Hz, 2H), 7.98 (d, J=8.4 Hz, 1H), 7.86 (dd, J=5.6, 3.2 Hz, 2H), 7.83 (d, J=8.4 Hz, 1H), 7.60 (ddd, J=8.4, 7.2, 1.2 Hz, 1H), 7.47 (ddd, J=8.0, 7.2, 1.2 Hz, 1H); ¹³C NMR (100 MHz, CDCl₃, δ): 166.1, 153.3, 147.1, 134.9, 131.8, 130.5, 128.5, 125.5, 124.3, 123.6, 120.2.

### JMM086

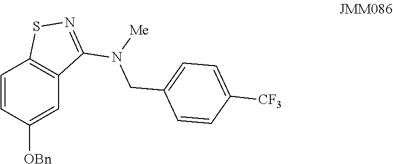 [SMILES: CN(Cc1ccc(C(F)(F)F)cc1)c1nsc2ccc(OCc3ccccc3)cc12]

### Procedure I (59%)

¹H NMR (400 MHz, CDCl₃, δ): 7.68 (d, J=8.8 Hz, 1H), 7.64 (d, J=8.0, 2H), 7.52 (d, J=8.0, 2H), 7.34-7.27 (m, 5H), 7.25 (d, J=2.0 Hz, 1H), 7.21 (dd, J=8.8, 2.0 Hz, 1H), 4.97 (s, 2H), 4.67 (s, 2H), 3.07 (s, 3H); ¹³C NMR (100 MHz, CDCl₃, δ): 163.0, 156.0, 146.1, 142.7, 136.4, 129.7 (q, J=32.2 Hz), 128.8, 128.6, 128.4, 128.0, 127.7, 127.2, 125.7 (q, J=3.8 Hz), 124.2 (q, J=270.5 Hz), 121.3, 119.1, 70.4, 57.1, 39.1; ¹⁹F NMR (376 MHz, CDCl₃, δ): −62.4.

### JMM087

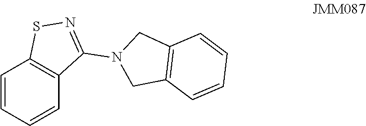 [SMILES: c1ccc2c(c1)CN(c1nsc3ccccc13)C2]

1,2-Bis(chloromethyl)benzene (0.2 mmol, 35 mg), DIPEA (0.5 mmol, 87 μL) and 3-amino benzoisothiazole (0.3 mmol, 45 mg) dissolved in toluene (2 mL) were added to a sealed tube before stirring at 110° C. under Argon overnight. The resulting mixture was cooled to room temperature and extracted with EtOAc. The combined organic phase was washed with brine, dried over Na₂SO₄ and concentrated in vacuo. The crude product was purified by column chromatography (EtOAc/Hexane). (12%)

¹H NMR (400 MHz, CDCl₃, δ): 8.27 (d, J=8.0 Hz, 1H), 7.80 (d, J=8.4 Hz, 1H), 7.47 (ddd, J=8.0, 7.2, 1.2 Hz, 1H), 7.39-7.32 (m, 5H), 5.26 (s, 4H); ¹³C NMR (100 MHz, CDCl₃, δ): 159.4, 153.6, 137.1, 127.45, 127.41, 126.9, 124.5, 123.7, 122.6, 120.5, 55.7.

### JMM088

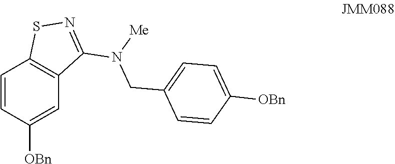 [SMILES: CN(Cc1ccc(OCc2ccccc2)cc1)c1nsc2ccc(OCc3ccccc3)cc12]

### Procedure I (62%)

¹H NMR (400 MHz, CDCl₃, δ): 7.67 (d, J=8.8 Hz, 1H), 7.45 (d, J=6.4 Hz, 2H), 7.41-7.37 (m, 2H), 7.35-7.29 (m, 9H), 7.20 (dd, J=8.8, 2.0 Hz, 1H), 7.01 (d, J=8.8 Hz, 2H), 5.09 (s, 2H), 4.95 (s, 2H), 4.57 (s, 2H), 3.05 (s, 3H); ¹³C NMR (100 MHz, CDCl₃, δ): 163.5, 158.2, 155.9, 145.9, 137.0, 136.5, 130.6, 128.8, 128.62, 128.59, 128.4, 128.0, 127.5, 127.4, 121.2, 119.1, 118.8, 115.1, 107.0, 70.3, 70.1, 56.9, 38.8.

### JMM089

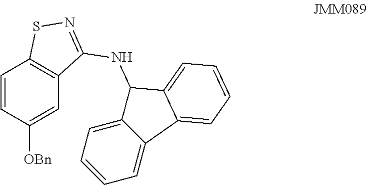 [SMILES: c1ccc(COc2ccc3snc(NC4c5ccccc5-c5ccccc54)c3c2)cc1]

### Procedure H (19%)

¹H NMR (400 MHz, CDCl₃, δ): 7.95-7.72 (m, 4H), 7.44-7.42 (m, 2H), 7.40-7.36 (m, 4H), 7.34-7.31 (m, 4H), 7.24 (dd, J=8.8, 2.4 Hz, 1H), 7.02 (d, J=2.4 Hz, 1H), 6.54 (d, J=9.2 Hz, 1H), 5.07 (d, J=9.2 Hz, 1H), 5.03 (s, 2H); ¹³C NMR (100 MHz, CDCl₃, δ): 158.3, 156.6, 145.5, 144.9, 140.6, 136.4, 128.7, 128.6, 128.2, 127.8, 127.5, 127.3, 125.5, 121.3, 119.9, 119.7, 103.9, 70.6, 58.1.

### JMM090

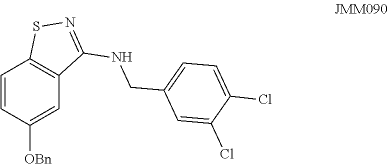 [SMILES: Clc1ccc(CNc2nsc3ccc(OCc4ccccc4)cc23)cc1Cl]

### Procedure G (13%)

¹H NMR (400 MHz, CDCl₃, δ): 7.67 (d, J=8.8 Hz, 1H), 7.52 (d, J=2.0 Hz, 1H), 7.46-7.35 (m, 6H), 7.27-7.23 (m, 2H), 7.13 (d, J=2.4 Hz, 1H), 5.12 (s, 2H), 5.12-5.09 (m, 1H), 4.73 (d, J=5.6 Hz, 2H); ¹³C NMR (100 MHz, CDCl₃, δ): 157.8, 156.6, 144.8, 139.5, 136.4, 132.6, 131.3, 130.6, 129.7, 128.7, 128.2, 127.5, 127.2, 127.0, 121.3, 119.5, 104.0, 70.7, 46.1.

### JMM091

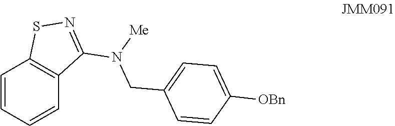 [SMILES: CN(Cc1ccc(OCc2ccccc2)cc1)c1nsc2ccccc12]

### Procedure I (91%)

¹H NMR (400 MHz, CDCl₃, δ): 7.92 (d, J=8.0 Hz, 1H), 7.81 (d, J=8.4 Hz, 1H), 7.47-7.38 (m, 5H), 7.36-7.33 (m, 3H), 7.28 (ddd, J=8.4, 6.8, 0.8 Hz, 1H), 7.00 (d, J=8.4 Hz, 2H), 5.08 (s, 2H), 4.70 (s, 2H), 3.11 (s, 3H); ¹³C NMR (100 MHz, CDCl₃ δ): 163.7, 158.2, 153.0, 137.0, 130.4, 128.7, 128.6, 128.0, 127.7, 127.5, 127.4, 124.2, 123.8, 120.6, 115.0, 70.1, 57.0, 38.7.

### JMM092

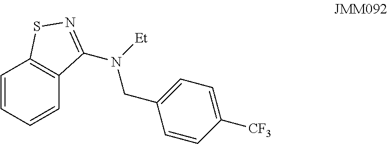 [SMILES: CCN(Cc1ccc(C(F)(F)F)cc1)c1nsc2ccccc12]

### Procedure I (33%)

¹H NMR (400 MHz, CDCl₃, δ): 7.85 (d, J=8.4 Hz, 1H), 7.81 (d, J=8.0 Hz, 1H), 7.61 (d, J=8.4 Hz, 2H), 7.54 (d, J=8.0 Hz, 2H), 7.45 (ddd, J=8.2, 6.8, 1.2 Hz, 1H), 7.29 (ddd, J=8.2, 6.8, 1.2 Hz, 1H), 4.84 (s, 2H), 3.63 (q, J=7.2 Hz, 2H), 1.29 (t, J=7.2 Hz, 3H); ¹³C NMR (100 MHz, CDCl₃, δ): 162.2, 153.2, 143.2, 129.4 (q, J=32.1 Hz), 127.7, 127.67, 127.4, 125.5 (q, J=3.8 Hz), 124.2 (q, J=270.1 Hz), 123.9, 123.8, 120.6, 54.1, 46.0, 13.0; ¹⁹F NMR (376 MHz, CDCl₃, δ): −62.3.

### JMM093

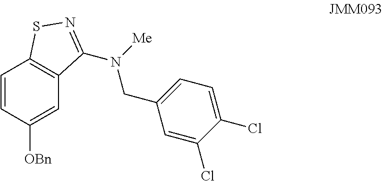 [SMILES: CN(Cc1ccc(Cl)c(Cl)c1)c1nsc2ccc(OCc3ccccc3)cc12]

### Procedure I (22%)

¹H NMR (400 MHz, CDCl₃, δ): 7.68 (dd, J=8.4, 0.8 Hz, 1H), 7.51 (d, J=2.0 Hz, 1H), 7.44 (d, J=8.0 Hz, 1H), 7.34-7.29 (m, 5H), 7.25-7.20 (m, 3H), 5.02 (s, 2H), 4.54 (s, 2H), 3.05 (s, 3H); ¹³C NMR (100 MHz, CDCl₃, δ): 162.9, 156.0, 146.1, 138.9, 136.4, 132.8, 131.3, 130.7, 129.4, 128.6, 128.4, 128.0, 127.2, 126.8, 121.3, 119.2, 106.9, 70.5, 56.5, 39.1.

### JMM094

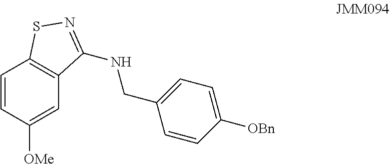 [SMILES: COc1ccc2snc(NCc3ccc(OCc4ccccc4)cc3)c2c1]

### Procedure G (28%)

¹H NMR (400 MHz, CDCl₃, δ): 7.64 (d, J=8.8 Hz, 1H), 7.45-7.34 (m, 7H), 7.14 (dd, J=8.8, 2.4 Hz, 1H), 7.00-6.96 (m, 3H), 5.07 (s, 2H), 4.99 (br s, 1H), 4.68 (d, J=4.4 Hz, 2H), 3.84 (s, 3H); ¹³C NMR (100 MHz, CDCl₃, δ): 162.7, 158.34, 158.31, 157.4, 144.3, 137.0, 131.3, 129.6, 128.6, 128.0, 127.5, 121.2, 119.0, 115.1, 102.5, 70.1, 55.7, 46.9.

### JMM095

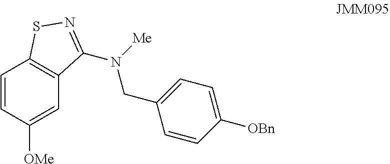 [SMILES: COc1ccc2snc(N(C)Cc3ccc(OCc4ccccc4)cc3)c2c1]

### Procedure I (46%)

¹H NMR (400 MHz, CDCl₃, δ): 7.66 (d, J=8.8 Hz, 1H), 7.46-7.33 (m, 7H), 7.28 (d, J=2.4 Hz, 1H), 7.11 (dd, J=8.8, 2.4 Hz, 1H), 6.99 (d, J=8.4 Hz, 2H), 5.08 (s, 2H), 4.64 (s, 2H), 3.69 (s, 3H), 3.08 (s, 3H); ¹³C NMR (100 MHz, CDCl₃, δ): 163.5, 158.1, 157.0, 145.7, 137.0, 130.6, 128.7, 128.6, 128.57, 128.0, 127.5, 121.1, 118.3, 115.1, 105.6, 70.1, 56.9, 55.5, 38.8.

### JMM096

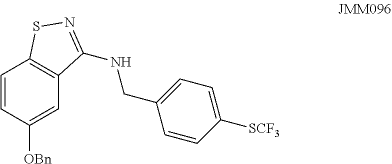

### Procedure G (14%)

¹H NMR (400 MHz, CDCl₃, δ): 7.67 (d, J=8.8 Hz, 1H), 7.63 (d, J=8.0 Hz, 2H), 7.49-7.33 (m, 7H), 7.24 (dd, J=8.8, 2.4 Hz, 1H), 7.14 (d, J=2.4 Hz, 1H), 5.14 (br s, 1H), 5.10 (s, 2H), 4.81 (d, J=4.8 Hz, 2H); ¹³C NMR (100 MHz, CDCl₃, δ): 158.0, 156.6, 144.8, 142.4, 136.6, 136.4, 129.6 (q, J=306.2 Hz), 128.9, 128.7, 128.2, 127.5, 127.0, 123.2 (q, J=1.9 Hz), 121.3, 119.5, 104.0, 70.7, 46.7; ¹⁹F NMR (376 MHz, CDCl₃, δ): −42.7.

### JMM097

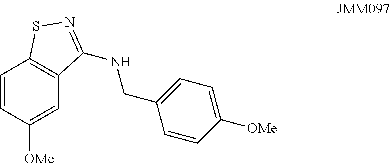 [SMILES: COc1ccc(CNc2nsc3ccc(OC)cc23)cc1]

### Procedure G (24%)

¹H NMR (400 MHz, CDCl₃, δ): 7.64 (d, J=8.8 Hz, 1H), 7.36 (d, J=8.4 Hz, 2H), 7.14 (dd, J=8.8, 2.4 Hz, 1H), 7.00 (d, J=2.4 Hz, 1H), 6.89 (d, J=8.4 Hz, 2H), 5.03 (br s, 1H), 4.68 (d, J=4.8 Hz, 2H), 3.83 (s, 3H), 3.80 (s, 3H); ¹³C NMR (100 MHz, CDCl₃, δ): 159.1, 158.4, 157.4, 144.3, 131.1, 129.6, 127.2, 121.1, 119.0, 114.1, 102.5, 55.7, 55.3, 46.9.

### JMM098

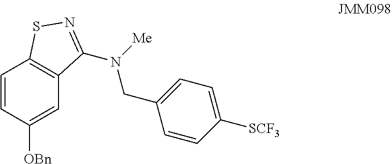

### Procedure I (52%)

¹H NMR (400 MHz, CDCl₃, δ): 7.69-7.67 (m, 3H), 7.47 (d, J=8.4 Hz, 2H), 7.33-7.27 (m, 5H), 7.24 (d, J=2.0 Hz, 1H), 7.21 (dd, J=8.8, 2.4 Hz, 1H), 4.97 (s, 2H), 4.64 (s, 2H), 3.08 (s, 3H); ¹³C NMR (100 MHz, CDCl₃, δ): 163.0, 156.0, 146.1, 141.9, 136.7, 136.4, 129.6 (q, J=306.1 Hz), 128.8, 128.6, 128.4, 128.0, 127.2, 123.1 (q, J=2.0 Hz), 121.3, 119.2, 106.9, 70.3, 57.0, 39.1; ¹⁹F NMR (376 MHz, CDCl₃, δ): −42.7.

### JMM099

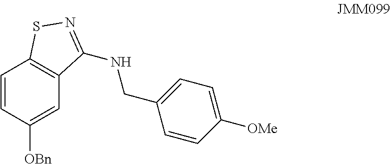 [SMILES: COc1ccc(CNc2nsc3ccc(OCc4ccccc4)cc23)cc1]

### Procedure G (21%)

¹H NMR (400 MHz, CDCl₃, δ): 7.66 (dd, J=8.8, 0.4 Hz, 1H), 7.45-7.34 (m, 7H), 7.22 (dd, J=8.8, 2.4 Hz, 1H), 7.10 (d, J=2.4 Hz, 1H), 6.91 (d, J=8.8 Hz, 2H), 5.07 (s, 2H), 4.96 (br s, 1H), 4.69 (d, J=5.2 Hz, 2H), 3.81 (s, 3H); ¹³C NMR (100 MHz, CDCl₃, δ): 159.1, 158.3, 156.5, 144.6, 136.5, 131.1, 129.5, 128.7, 128.2, 127.5, 127.2, 121.2, 119.4, 114.1, 104.0, 70.6, 55.4, 46.9.

### JMM100

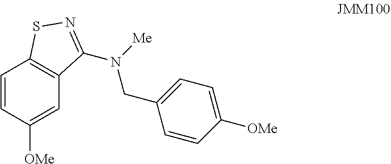 [SMILES: COc1ccc(CN(C)c2nsc3ccc(OC)cc23)cc1]

### Procedure I (89%)

¹H NMR (400 MHz, CDCl₃, δ): 7.66 (d, J=8.8 Hz, 1H), 7.34 (d, J=8.8 Hz, 2H), 7.29 (d, J=2.4 Hz, 1H), 7.11 (dd, J=8.8, 2.4 Hz, 1H), 6.92 (d, J=8.4 Hz, 2H), 4.64 (s, 2H), 3.82 (s, 3H), 3.70 (s, 3H), 3.08 (s, 3H); ¹³C NMR (100 MHz, CDCl₃, δ): 163.5, 158.9, 157.0, 145.7, 130.3, 128.7, 128.6, 121.1, 118.3, 114.1, 105.7, 56.9, 55.5, 55.3, 38.8.

### JMM101S

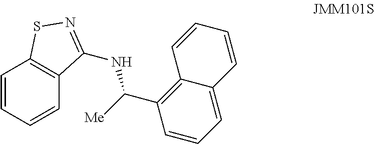 [SMILES: C[C@H](Nc1nsc2ccccc12)c1cccc2ccccc12]

A mixture of 3-chloro benzoisothiazole (1 mmol, 169 mg), Pd(OAc)₂ (10 mol %), Xantphos (10 mol %), Cs₂CO₃ (1.5 mmol, 488 mg) and aniline (1 mmol, 160 uL) was added 1,4-dioxane under Ar. The reaction mixture was heated at 100° C. overnight and after it cooled to room temperature, filter through the celite. The solution was concentrated and purified on silicon gel column chromatography (EtOAc/Hexane) (11%).

¹H NMR (400 MHz, CDCl₃, δ): 8.20-8.18 (m, 1H), 7.89-7.87 (m, 1H), 7.82-7.77 (m, 2H), 7.66 (d, J=7.2 Hz, 1H), 7.59 (d, J=8.0 Hz, 1H), 7.51-7.45 (m, 4H), 7.29 (ddd, J=8.0, 7.2, 1.2 Hz, 1H), 6.13-6.06 (quintet, J=6.8 Hz, 1H), 5.24 (d, J=6.8 Hz, 1H), 1.84 (d, J=6.4 Hz, 3H); ¹³C NMR (100 MHz, CDCl₃, δ): 157.7, 151.6, 139.4, 134.1, 131.3, 128.8, 128.2, 128.0, 126.5, 126.4, 125.8, 125.4, 123.8, 123.6, 122.4, 121.0, 120.4, 48.3, 21.3.

### JMM102R

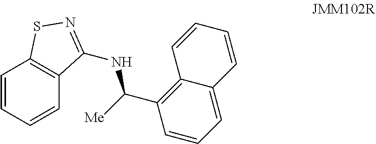 [SMILES: C[C@@H](Nc1nsc2ccccc12)c1cccc2ccccc12]

A mixture of 3-chloro benzoisothiazole (1 mmol, 169 mg), Pd(OAc)₂ (10 mol %), Xantphos (10 mol %), Cs₂CO₃ (1.5 mmol, 488 mg) and aniline (1 mmol, 160 uL) was added 1,4-dioxane under Ar. The reaction mixture was heated at 100° C. overnight and after it cooled to room temperature, filter through the celite. The solution was concentrated and purified on silicon gel column chromatography (EtOAc/Hexane) (6%).

¹H NMR (400 MHz, CDCl₃, δ): 8.21-8.19 (m, 1H), 7.89-7.87 (m, 1H), 7.83-7.78 (m, 2H), 7.66 (d, J=7.2 Hz, 1H), 7.59 (d, J=8.0 Hz, 1H), 7.52-7.45 (m, 4H), 7.30 (ddd, J=8.0, 7.2, 1.2 Hz, 1H), 6.14-6.08 (quintet, J=6.8 Hz, 1H), 5.21 (d, J=6.8 Hz, 1H), 1.85 (d, J=6.8 Hz, 3H); ¹³C NMR (100 MHz, CDCl₃, δ): 157.7, 151.6, 139.4, 134.0, 131.3, 128.8, 128.2, 128.0, 126.45, 126.43, 125.8, 125.4, 123.8, 123.6, 122.4, 121.0, 120.4, 48.3, 21.3.

### JMM103

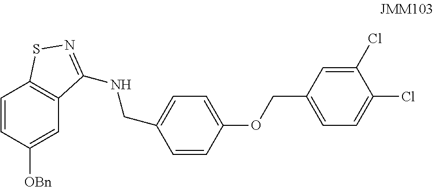 [SMILES: Clc1ccc(COc2ccc(CNc3nsc4ccc(OCc5ccccc5)cc34)cc2)cc1Cl]

### Procedure G (18%)

¹H NMR (400 MHz, CDCl₃, δ): 7.66 (d, J=8.8 Hz, 1H), 7.53 (d, J=2.0 Hz, 1H), 7.46-7.34 (m, 8H), 7.25 (dd, J=8.0, 2.0 Hz, 1H), 7.22 (dd, J=8.8, 2.0 Hz, 1H), 7.10 (d, J=2.4 Hz, 1H), 6.93 (d, J=8.4 Hz, 2H), 5.07 (s, 2H), 5.01 (s, 2H), 4.99 (br s, 1H), 4.69 (d, J=5.2 Hz, 2H); ¹³C NMR (100 MHz, CDCl₃, δ): 158.3, 157.8, 156.5, 144.6, 137.3, 136.4, 132.8, 132.0, 131.9, 130.6, 129.6, 129.2, 128.7, 128.2, 127.5, 127.2, 126.5, 121.2, 119.4, 115.0, 104.1, 70.6, 68.6, 46.8.

### JMM104

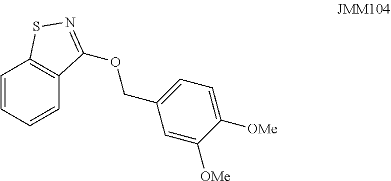 [SMILES: COc1ccc(COc2nsc3ccccc23)cc1OC]

A pressure flask was charged with Pd(OAc)₂ (0.02 mmol, 4.5 mg), TrixiePhos (0.025 mmol, 10 mg) and Cs₂CO₃ (1.5 mmol, 488 mg), 3-chlorobenzoisothiazole (1 mmol, 170 mg) and 3,4-dimethoxybenzyl alcohol (2 mmol, 291 uL). Toluene (2.5 mL) was added via syringe. The flask was then evacuated and back-filled with Argon and sealed. The flask was placed at 100° C. overnight. The reaction mixture was cooled to room temperature and filtered through a pad of celite and concentrated. The crude product was purified by flash chromatography (EtOAc/Hexane). (13%)

¹H NMR (400 MHz, CDCl₃, δ): 7.59 (dd, J=8.0, 1.2 Hz, 1H), 7.45-7.41 (m, 1H), 7.36 (dd, J=8.0, 0.8 Hz, 1H), 7.27-7.23 (m, 1H), 6.84-6.79 (m, 2H), 6.73 (d, J=8.4 Hz, 1H), 4.16 (s, 2H), 3.830 (s, 3H), 3.826 (s, 3H); ¹³C NMR (100 MHz, CDCl₃, δ): 149.0, 148.5, 140.7, 133.6, 132.7, 131.0, 128.3, 126.7, 121.2, 117.2, 114.7, 111.9, 111.0, 55.90, 55.88, 38.9.

### N-([1,1′-Biphenyl]-4-ylmethyl)-5-(benzyloxy)benzo[d]isothiazol-3-amine, JMM105

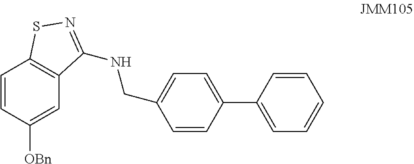 [SMILES: c1ccc(COc2ccc3snc(NCc4ccc(-c5ccccc5)cc4)c3c2)cc1]

### Procedure G (35%)

¹H NMR (400 MHz, CDCl₃, δ): 7.68 (d, J=8.8 Hz, 1H), 7.61-7.58 (m, 4H), 7.52 (d, J=8.0 Hz, 2H), 7.47-7.41 (m, 5H), 7.40-7.34 (m, 3H), 7.24 (dd, J=8.8, 2.4 Hz, 1H), 7.14 (d, J=2.0 Hz, 1H), 5.10 (s, 2H), 5.07 (t, J=5.2 Hz, 1H), 4.81 (d, J=5.2 Hz, 2H); ¹³C NMR (100 MHz, CDCl₃, δ): 158.3, 156.6, 144.7, 140.8, 140.6, 138.1, 136.5, 128.8, 128.7, 128.6, 128.2, 127.54, 127.46, 127.35, 127.2, 127.1, 121.3, 119.4, 104.0, 70.7, 47.1.

### 5-(Benzyloxy)-N-(4-(naphthalen-1-ylmethoxy)benzyl)benzo[d]isothiazol-3-amine, JMM106

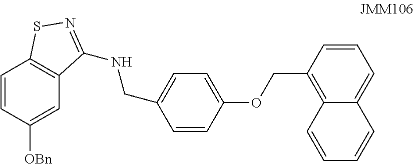 [SMILES: c1ccc(COc2ccc3snc(NCc4ccc(OCc5cccc6ccccc56)cc4)c3c2)cc1]

### Procedure G (27%)

¹H NMR (400 MHz, CDCl₃, δ): 8.07-8.04 (m, 1H), 7.92-7.85 (m, 2H), 7.66 (d, J=8.8 Hz, 1H), 7.60 (d, J=6.8 Hz, 1H), 7.55-7.52 (m, 2H), 7.49-7.35 (m, 8H), 7.22 (dd, J=8.8, 2.0 Hz, 1H), 7.12 (d, J=2.4 Hz, 1H), 7.05 (d, J=8.8 Hz, 2H), 5.49 (s, 2H), 5.04-5.01 (m, 1H), 4.71 (d, J=5.2 Hz, 2H); ¹³C NMR (100 MHz, CDCl₃, δ): 158.37, 158.36, 156.5, 144.6, 136.5, 133.8, 132.2, 131.5, 129.6, 129.1, 128.8, 128.7, 128.2, 127.6, 127.5, 127.2, 126.6, 126.5, 125.9, 125.3, 123.7, 121.2, 119.4, 115.1, 104.1, 70.6, 68.8, 46.9.

### 5-(Benzyloxy)-N-(4-methoxybenzyl)-N-methylbenzo[d]isothiazol-3-amine, JMM107

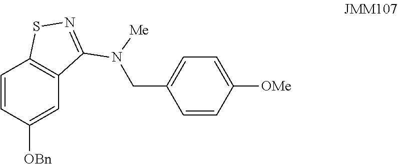 [SMILES: COc1ccc(CN(C)c2nsc3ccc(OCc4ccccc4)cc23)cc1]

### Procedure I (41%)

¹H NMR (400 MHz, CDCl₃, δ): 7.66 (d, J=8.4 Hz, 1H), 7.35-7.29 (m, 8H), 7.19 (dd, J=8.8, 2.4 Hz, 1H), 6.93 (d, J=8.8 Hz, 2H), 4.96 (s, 2H), 4.56 (s, 2H), 3.83 (s, 3H), 3.04 (s, 3H); ¹³C NMR (100 MHz, CDCl₃, δ): 163.5, 160.0, 155.9, 145.9, 136.5, 130.3, 128.8, 128.60, 128.58, 128.0, 127.4, 121.2, 119.1, 114.1, 107.0, 70.3, 56.9, 55.3, 38.7;

### 5-(Benzyloxy)-N-(4-((4-chlorobenzyl)oxy)benzyl)-N-methylbenzo[d]isothiazol-3-amine, JMM108

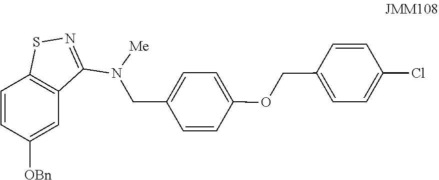 [SMILES: CN(Cc1ccc(OCc2ccc(Cl)cc2)cc1)c1nsc2ccc(OCc3ccccc3)cc12]

### Procedure I (32%)

¹H NMR (400 MHz, CDCl₃, δ): 7.66 (d, J=8.8 Hz, 1H), 7.38-7.30 (m, 12H), 7.19 (dd, J=8.8, 2.4 Hz, 1H), 6.98 (d, J=8.4 Hz, 2H), 5.05 (s, 2H), 4.95 (s, 2H), 4.57 (s, 2H), 3.05 (s, 3H); ¹³C NMR (100 MHz, CDCl₃, δ): 163.4, 157.9, 155.9, 145.9, 136.5, 135.5, 133.8, 130.9, 129.9, 128.79, 128.77, 128.65, 128.62, 128.0, 127.4, 121.2, 119.1, 115.0, 107.0, 70.3, 69.3, 56.9, 38.8.

### 5-(Benzyloxy)-N-(4-((3,4-dichlorobenzyl)oxy)benzyl)-N-methylbenzo[d]isothiazol-3-amine, JMM109

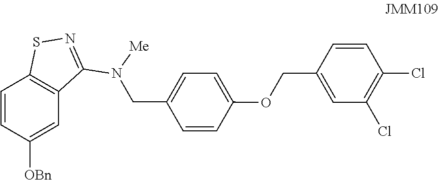 [SMILES: CN(Cc1ccc(OCc2ccc(Cl)c(Cl)c2)cc1)c1nsc2ccc(OCc3ccccc3)cc12]

### Procedure I (15%)

¹H NMR (400 MHz, CDCl₃, δ): 6.67 (d, J=8.8 Hz, 1H), 7.55 (d, J=2.0 Hz, 1H), 7.44 (d, J=8.0 Hz, 1H), 7.34-7.27 (m, 9H), 7.19 (dd, J=8.8, 2.4 Hz, 1H), 6.97 (d, J=8.8 Hz, 2H), 5.02 (s, 2H), 4.95 (s, 2H), 4.57 (s, 2H), 3.04 (s, 3H); ¹³C NMR (100 MHz, CDCl₃, δ): 163.4, 157.7, 155.9, 145.9, 137.3, 136.5, 132.8, 132.0, 131.1, 130.6, 129.2, 128.7, 128.62, 128.59, 128.1, 127.4, 126.5, 121.2, 119.1, 115.0, 107.0, 70.3, 68.6, 56.9, 38.8.

### N-Methyl-N-(naphthalen-1-ylmethyl)benzo[d]isothiazol-3-amine, JMM110

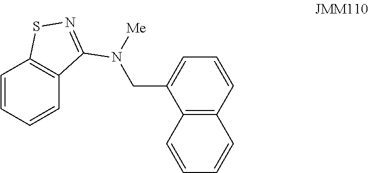 [SMILES: CN(Cc1cccc2ccccc12)c1nsc2ccccc12]

### Procedure I (79%)

¹H NMR (400 MHz, CDCl₃, δ): 8.00-7.98 (m, 1H), 7.94 (dd, J=7.2, 2.4 Hz, 1H), 7.85 (d, J=8.4 Hz, 1H), 7.81 (d, J=8.4 Hz, 1H), 7.76-7.72 (m, 2H), 7.56-7.49 (m, 3H), 7.41 (ddd, J=8.0, 7.2, 1.2 Hz, 1H), 7.16 (ddd, J=8.4, 7.2, 1.2 Hz, 1H), 5.23 (s, 2H), 3.24 (s, 3H); ¹³C NMR (100 MHz, CDCl₃, δ): 163.6, 153.1, 133.9, 133.4, 131.3, 128.9, 128.0, 127.6, 127.4, 126.3, 125.9, 125.7, 124.8, 124.3, 123.9, 122.9, 120.5, 55.4, 39.4.

### 5-(Benzyloxy)-N-(4-((2,4-difluorobenzyl)oxy)benzyl)benzo[d]isothiazol-3-amine, JMM111

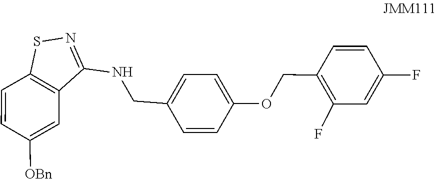 [SMILES: Fc1ccc(COc2ccc(CNc3nsc4ccc(OCc5ccccc5)cc34)cc2)c(F)c1]

### Procedure G (12%)

¹H NMR (400 MHz, CDCl₃, δ): 7.66 (d, J=8.8 Hz, 1H), 7.49-7.34 (m, 8H), 7.22 (dd, J=8.8, 2.0 Hz, 1H), 7.01 (d, J=2.4 Hz, 1H), 6.97 (d, J=8.8 Hz, 2H), 6.93-6.82 (m, 2H), 5.07 (s, 4H), 5.00 (m, 1H), 4.69 (d, J=4.4 Hz, 2H); ¹³C NMR (100 MHz, CDCl₃, δ): 162.8 (dd, J=247.8, 11.9 Hz), 160.6 (dd, J=248.2, 11.9 Hz), 158.3, 157.9, 156.5, 144.6, 136.5, 131.8, 130.8 (dd, J=9.8, 5.5 Hz), 129.6, 128.7, 128.2, 127.5, 127.2, 121.2, 120.1 (dd, J=14.5, 3.7 Hz), 119.4, 115.0, 111.5 (dd, J=21.0, 3.6 Hz), 104.1, 103.9 (dd, J=25.3, 25.2 Hz), 70.6, 63.3 (d, J=3.8 Hz), 46.9; ¹⁹F NMR (376 MHz, CDCl₃, δ): −110.0 (d, J=7.52 Hz), −114.3 (d, J=7.52 Hz).

### 5-(Benzyloxy)-N-(4-((4-chlorobenzyl)oxy)benzyl)benzo[d]isothiazol-3-amine, JMM112

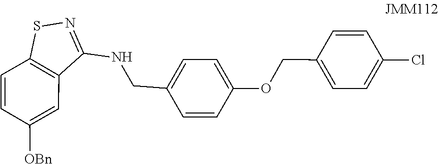 [SMILES: Clc1ccc(COc2ccc(CNc3nsc4ccc(OCc5ccccc5)cc34)cc2)cc1]

### Procedure G (19%)

¹H NMR (400 MHz, DMSO-d₆, δ): 7.85 (d, J=2.4 Hz, 1H), 7.77 (d, J=8.8 Hz, 1H), 7.69 (t, J=5.6 Hz, 1H), 7.47-7.37 (m, 9H), 7.29 (d, J=8.8 Hz, 2H), 7.19 (dd, J=8.8, 2.4 Hz, 1H), 6.93 (d, J=8.8 Hz, 2H), 5.09 (s, 2H), 5.05 (s, 2H), 4.51 (d, J=5.6 Hz, 2H); ¹³C NMR (100 MHz, DMSO-d₆, δ): 159.2, 157.5, 156.5, 143.6, 137.2, 136.7, 132.8, 132.7, 129.8, 129.4, 128.9, 128.9, 128.43, 128.36, 127.9, 121.7, 119.4, 115.0, 106.0, 70.2, 68.7, 45.7.

### 5-(Benzyloxy)-N-(4-((4-bromobenzyl)oxy)benzyl)benzo[d]isothiazol-3-amine, JMM113

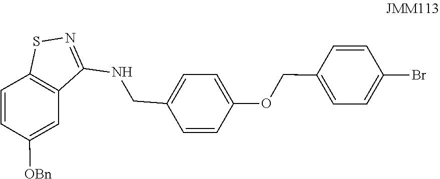 [SMILES: Brc1ccc(COc2ccc(CNc3nsc4ccc(OCc5ccccc5)cc34)cc2)cc1]

### Procedure G (21%)

¹H NMR (400 MHz, DMSO-d₆, δ): 7.85 (d, J=2.4 Hz, 1H), 7.77 (d, J=8.8 Hz, 1H), 7.69 (t, J=5.6 Hz, 1H), 7.54 (d, J=8.0 Hz, 2H), 7.47-7.45 (m, 2H), 7.39-7.42 (m, 5H), 7.28 (d, J=8.4 Hz, 2H), 7.19 (dd, J=6.8, 2.0 Hz, 1H), 6.92 (d, J=8.8 Hz, 2H), 5.09 (s, 2H), 5.03 (s, 2H), 4.51 (d, J=5.6 Hz, 2H); ¹³C NMR (100 MHz, DMSO-d₆, δ): 159.2, 157.5, 156.5, 143.6, 137.2, 137.1, 132.8, 131.8, 130.1, 129.4, 128.9, 128.43, 128.36, 127.9, 121.7, 121.3, 119.4, 115.1, 106.0, 70.2, 68.8, 45.7.

### 5-(Benzyloxy)-N-(4-((2-methylbenzyl)oxy)benzyl)benzo[d]isothiazol-3-amine, JMM114

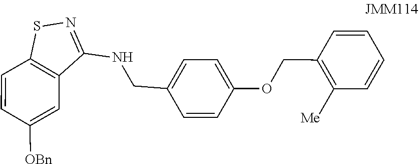 [SMILES: Cc1ccccc1COc1ccc(CNc2nsc3ccc(OCc4ccccc4)cc23)cc1]

### Procedure G (18%)

¹H NMR (400 MHz, CDCl₃, δ): 7.66 (d, J=8.8 Hz, 1H), 7.45-7.35 (m, 8H), 7.27-7.21 (m, 4H), 7.11 (d, J=2.0 Hz, 1H), 7.00 (d, J=8.4 Hz, 2H), 5.08 (s, 2H), 5.04 (s, 2H), 4.99 (br s, 1H), 4.70 (d, J=4.8 Hz, 2H), 2.39 (s, 3H); ¹³C NMR (100 MHz, CDCl₃, δ): 158.5, 158.3, 156.5, 144.6, 136.7, 136.5, 134.7, 131.3, 130.5, 129.6, 128.7, 128.6, 128.3, 128.2, 127.6, 127.2, 126.1, 121.2, 119.4, 115.0, 104.0, 70.6, 68.7, 46.9, 18.9.

### 5-(Benzyloxy)-N-(4-phenoxybenzyl)benzo[d]isothiazol-3-amine, JMM115

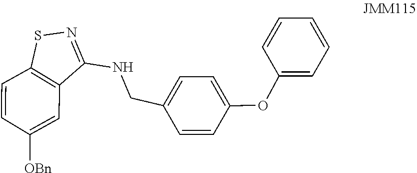 [SMILES: c1ccc(COc2ccc3snc(NCc4ccc(Oc5ccccc5)cc4)c3c2)cc1]

### Procedure B (25%)

¹H NMR (400 MHz, CDCl₃, δ): 7.66 (d, J=8.8 Hz, 1H), 7.45-7.32 (m, 9H), 7.23 (dd, J=8.8, 2.0 Hz, 1H), 7.14-7.11 (m, 2H), 7.01 (m, 4H), 5.08 (s, 2H), 5.08-5.05 (m, 1H), 4.73 (d, J=5.2 Hz, 2H); ¹³C NMR (100 MHz, CDCl₃, δ): 158.3, 157.2, 156.7, 156.6, 144.6, 136.5, 133.8, 129.8, 129.6, 128.7, 128.2, 127.6, 127.2, 123.4, 121.3, 119.4, 119.0, 118.9, 104.1, 70.6, 46.8.

### 5-(Benzyloxy)-N-((4-(benzyloxy)naphthalen-1-yl)methyl)benzo[d]isothiazol-3-amine, JMM116

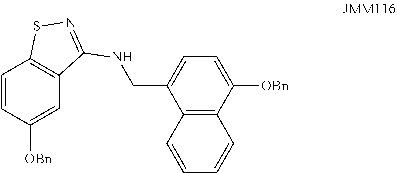 [SMILES: c1ccc(COc2ccc3snc(NCc4ccc(OCc5ccccc5)c5ccccc45)c3c2)cc1]

### Procedure B (31%)

¹H NMR (400 MHz, CDCl₃, δ): 8.46-8.44 (m, 1H), 8.11-8.09 (m, 1H), 7.67 (d, J=8.8 Hz, 1H), 7.56-7.52 (m, 4H), 7.48-7.42 (m, 3H), 7.39-7.31 (m, 6H), 7.21 (dd, J=8.8, 2.4 Hz, 1H), 7.01 (d, J=2.4 Hz, 1H), 6.85 (d, J=7.6 Hz, 1H), 5.28 (s, 2H), 5.10 (d, J=4.8 Hz, 2H), 4.99 (s, 2H), 4.95 (t, J=4.8 Hz, 1H); ¹³C NMR (100 MHz, CDCl₃, δ): 158.3, 156.5, 154.8, 144.6, 137.0, 136.4, 132.8, 128.7, 128.6, 128.1, 128.0, 127.53, 127.46, 127.4, 127.2, 127.1, 126.4, 126.2, 125.4, 123.9, 122.9, 121.2, 119.5, 104.6, 104.0, 70.6, 70.1, 45.5.

### 5-(Benzyloxy)-N-(4-((4-fluorobenzyl)oxy)benzyl)benzo[d]isothiazol-3-amine, JMM117

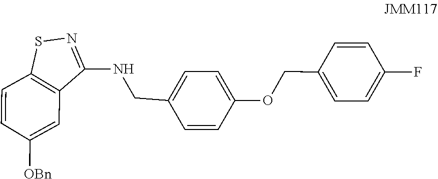 [SMILES: Fc1ccc(COc2ccc(CNc3nsc4ccc(OCc5ccccc5)cc34)cc2)cc1]

### Procedure B (26%)

¹H NMR (400 MHz, DMSO-d₆, δ): 7.85 (d, J=2.4 Hz, 1H), 7.77 (d, J=8.8 Hz, 1H), 7.70 (t, J=5.6 Hz, 1H), 7.47-7.42 (m, 4H), 7.39-7.35 (m, 2H), 7.33-7.28 (m, 3H), 7.21-7.14 (m, 3H), 6.93 (d, J=8.4 Hz, 2H), 5.09 (s, 2H), 5.03 (s, 2H), 4.52 (d, J=5.6 Hz, 2H); ¹³C NMR (100 MHz, DMSO-d₆, δ): 162.1 (d, J=241.9 Hz), 159.2, 157.6, 156.5, 143.6, 137.2, 133.9 (d, J=3.0 Hz), 132.7, 130.3 (d, J=8.1 Hz), 129.4, 128.9, 128.43, 128.36, 127.9, 121.7, 119.4, 115.7 (d, J=21.3 Hz), 115.0, 106.0, 70.2, 68.9, 45.8; ¹⁹F NMR (376 MHz, DMSO-d₆, δ): −114.6.

### 5-(Benzyloxy)-N-(4-((2-fluorobenzyl)oxy)benzyl)benzo[d]isothiazol-3-amine, JMM118

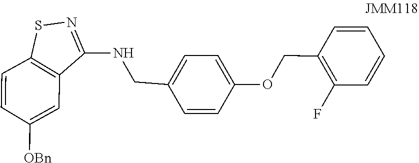 [SMILES: Fc1ccccc1COc1ccc(CNc2nsc3ccc(OCc4ccccc4)cc23)cc1]

### Procedure B (18%)

¹H NMR (400 MHz, DMSO-d₆, δ): 7.85 (d, J=2.4 Hz, 1H), 7.77 (d, J=8.8 Hz, 1H), 7.70 (t, J=5.6 Hz, 1H), 7.50 (ddd, J=7.8, 7.6, 2.0 Hz, 1H), 7.47-7.45 (m, 2H), 7.39-7.35 (m, 3H), 7.33-7.29 (m, 3H), 7.23-7.16 (m, 3H), 6.96 (d, J=8.8 Hz, 2H), 5.09 (s, 2H), 5.08 (s, 2H), 4.52 (d, J=5.6 Hz, 2H); ¹³C NMR (100 MHz, DMSO-d₆, δ): 160.8 (d, J=244.3 Hz), 159.2, 157.6, 156.5, 143.6, 137.2, 132.9, 131.0 (d, J=4.2 Hz), 130.8 (d, J=8.1 Hz), 129.4, 128.9, 128.43, 128.36, 127.9, 125.0 (d, J=3.4 Hz), 124.4 (d, J=14.5 Hz), 121.7, 119.4, 115.8 (d, J=20.8 Hz), 114.9, 106.0, 70.2, 64.0 (d, J=3.8 Hz), 45.8; ¹⁹F NMR (376 MHz, DMSO-d₆, δ): −118.4.

### 5-(Benzyloxy)-N-(thiophen-2-ylmethyl)benzo[d]isothiazol-3-amine, JMM119

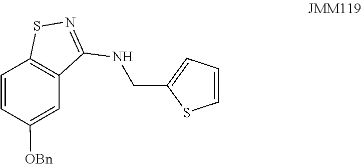 [SMILES: c1ccc(COc2ccc3snc(NCc4cccs4)c3c2)cc1]

### Procedure B (29%)

¹H NMR (400 MHz, CDCl₃, δ): 7.67 (d, J=8.8 Hz, 1H), 7.46-7.35 (m, 5H), 7.26 (dd, J=5.2, 1.2 Hz, 1H), 7.23 (dd, J=8.8, 2.4 Hz, 1H), 7.10 (d, J=2.4 Hz, 2H), 6.99 (dd, J=4.8, 3.2 Hz, 1H), 5.10 (s, 2H), 5.03 (t, J=5.6 Hz, 1H), 4.95 (d, J=5.2 Hz, 2H); ¹³C NMR (100 MHz, CDCl₃, δ): 157.8, 156.6, 144.8, 141.8, 136.4, 128.7, 128.2, 127.5, 127.1, 126.9, 126.2, 125.2, 121.2, 119.4, 104.0, 70.7, 42.1.

### N-(4-benzylbenzyl)-5-(benzyloxy)benzo[d]isothiazol-3-amine, JMM120

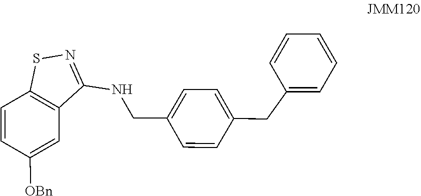 [SMILES: c1ccc(COc2ccc3snc(NCc4ccc(Cc5ccccc5)cc4)c3c2)cc1]

### Procedure B (34%)

¹H NMR (400 MHz, CDCl₃, δ): 7.66 (d, J=8.8 Hz, 1H), 7.45-7.28 (m, 9H), 7.23-7.20 (m, 6H), 7.09 (d, J=2.4 Hz, 1H), 5.08 (s, 2H), 4.94 (t, J=5.2 Hz, 1H), 4.72 (d, J=5.2 Hz, 2H), 3.99 (s, 2H); ¹³C NMR (100 MHz, CDCl₃, δ): 158.3, 156.5, 144.7, 141.0, 140.7, 136.7, 136.5, 129.2, 128.9, 128.7, 128.5, 128.4, 128.2, 127.5, 127.1, 126.2, 121.2, 119.4, 104.0, 70.6, 47.2, 41.7.

### 5-(Benzyloxy)-N-(4-((4-(trifluoromethyl)benzyl)oxy)benzyl)benzo[d]isothiazol-3-amine, JMM121

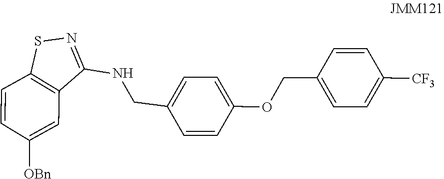 [SMILES: FC(F)(F)c1ccc(COc2ccc(CNc3nsc4ccc(OCc5ccccc5)cc34)cc2)cc1]

### Procedure B (28%)

¹H NMR (400 MHz, CDCl₃, δ): 7.67-7.64 (m, 3H), 7.55 (d, J=8.0 Hz, 2H), 7.45-7.32 (m, 7H), 7.22 (d, J=8.4 Hz, 1H), 7.11 (m, 1H), 6.96 (d, J=7.6 Hz, 2H), 5.13 (s, 2H), 5.09 (s, 2H), 4.98 (br s, 1H), 4.71 (s, 2H); ¹³C NMR (100 MHz, CDCl₃, δ): 158.2, 157.9, 156.5, 144.6, 141.1, 136.4, 131.8, 130.2 (q, J=32.5 Hz), 129.6, 128.7, 128.2, 127.5, 127.3, 127.1, 125.6 (q, J=3.7 Hz), 124.1 (q, J=270.4 Hz), 121.3, 119.4, 115.0, 104.1, 70.7, 69.2, 46.9; ¹⁹F NMR (376 MHz, CDCl₃, δ): −62.5.

### N-(4-(Benzylamino)benzyl)-5-(benzyloxy)benzo[d]isothiazol-3-amine, JMM122

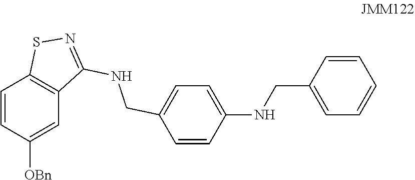 [SMILES: c1ccc(CNc2ccc(CNc3nsc4ccc(OCc5ccccc5)cc34)cc2)cc1]

### Procedure B (34%)

¹H NMR (400 MHz, CDCl₃, δ): 7.65 (d, J=8.8 Hz, 1H), 7.45-7.32 (m, 9H), 7.30-7.25 (m, 3H), 7.21 (dd, J=8.8, 2.4 Hz, 1H), 7.07 (d, J=2.4 Hz, 1H), 6.65 (d, J=8.4 Hz, 2H), 5.08 (s, 2H), 4.85 (br s, 1H), 4.62 (d, J=4.8 Hz, 2H), 4.35 (s, 2H), 4.18 (br s, 1H); ¹³C NMR (100 MHz, CDCl₃, δ): 158.4, 156.5, 147.7, 144.5, 139.3, 136.5, 129.6, 128.7, 128.6, 128.2, 127.6, 127.5, 127.4, 127.3, 127.2, 121.2, 119.4, 112.9, 104.0, 70.6, 48.3, 47.2.

### 5-(Benzyloxy)-N-(4-((2,4,6-trimethylbenzyl)oxy)benzyl)benzo[d]isothiazol-3-amine, JMM123

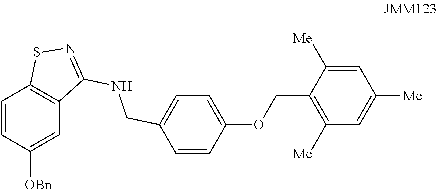 [SMILES: Cc1cc(C)c(COc2ccc(CNc3nsc4ccc(OCc5ccccc5)cc34)cc2)c(C)c1]

### Procedure B (40%)

¹H NMR (400 MHz, DMSO-d₆, δ): 7.86 (d, J=2.4 Hz, 1H), 7.77 (d, J=8.8 Hz, 1H), 7.70 (t, J=5.6 Hz, 1H), 7.47-7.45 (m, 2H), 7.39-7.35 (m, 2H), 7.33-7.29 (m, 3H), 7.19 (dd, J=8.8, 2.4 Hz, 1H), 6.96 (d, J=8.8 Hz, 2H), 6.84 (s, 2H), 5.10 (s, 2H), 4.93 (s, 2H), 4.52 (d, J=6.0 Hz, 2H), 2.23 (s, 6H), 2.19 (s, 3H); ¹³C NMR (100 MHz, DMSO-d₆, δ): 159.2, 158.5, 156.5, 143.6, 138.0, 137.8, 137.2, 132.6, 130.5, 129.4, 129.1, 128.9, 128.43, 128.36, 127.9, 121.7, 119.4, 114.8, 106.0, 70.2, 64.7, 45.8, 21.1, 19.5.

### 5-(Benzyloxy)-N-(4-(phenoxymethyl)benzyl)benzo[d]isothiazol-3-amine, JMM124

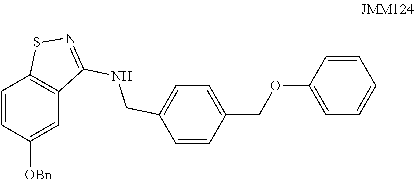 [SMILES: c1ccc(COc2ccc3snc(NCc4ccc(COc5ccccc5)cc4)c3c2)cc1]

### Procedure B (51%)

¹H NMR (400 MHz, CDCl₃, δ): 7.67 (d, J=8.8 Hz, 1H), 7.48-7.28 (m, 11H), 7.23 (dd, J=8.8, 2.0 Hz, 1H), 7.11 (d, J=2.4 Hz, 1H), 7.00-6.95 (m, 3H), 5.09 (s, 2H), 5.07 (s, 2H), 5.01 (t, J=5.2 Hz, 1H), 4.77 (d, J=5.2 Hz, 2H); ¹³C NMR (100 MHz, CDCl₃, δ): 158.7, 158.2, 156.6, 144.7, 138.8, 136.5, 129.5, 128.7, 128.4, 128.2, 128.0, 127.5, 127.1, 121.3, 121.0, 119.5, 114.8, 104.0, 70.6, 69.6, 47.2 (one low-field carbon not observed).

### N-(4-(Benzyl(methyl)amino)benzyl)-5-(benzyloxy)benzo[d]isothiazol-3-amine, JMM125

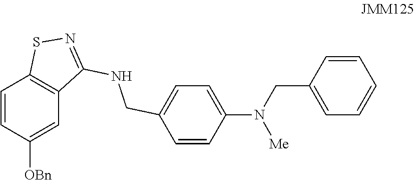 [SMILES: CN(Cc1ccccc1)c1ccc(CNc2nsc3ccc(OCc4ccccc4)cc23)cc1]

### Procedure B (42%)

¹H NMR (400 MHz, CDCl₃, δ): 7.65 (d, J=8.8 Hz, 1H), 7.45-7.29 (m, 9H), 7.26-7.20 (m, 4H), 7.07 (d, J=2.4 Hz, 1H), 6.75 (d, J=8.8 Hz, 2H), 5.07 (s, 2H), 4.85 (t, J=4.8 Hz 1H), 4.63 (d, J=5.2 Hz, 2H), 4.55 (s, 2H), 3.06 (s, 3H); ¹³C NMR (100 MHz, CDCl₃, δ): 158.5, 156.5, 149.3, 144.5, 138.9, 136.5, 129.6, 128.7, 128.6, 128.2, 127.5, 127.2, 126.9, 126.7, 126.4, 121.2, 119.4, 112.4, 104.0, 70.6, 56.6, 47.1, 38.8.

### 5-(Benzyloxy)-N-(4-(dibenzylamino)benzyl)benzo[d]isothiazol-3-amine, JMM126

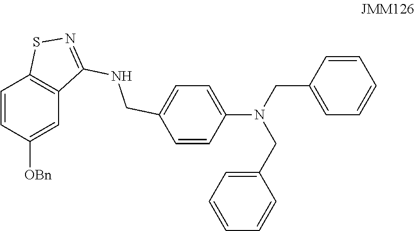 [SMILES: c1ccc(COc2ccc3snc(NCc4ccc(N(Cc5ccccc5)Cc5ccccc5)cc4)c3c2)cc1]

¹H NMR (400 MHz, CDCl₃, δ): 7.65 (d, J=8.8 Hz, 1H), 7.45-7.31 (m, 9H), 7.28-7.24 (m, 8H), 7.20 (dd, J=8.8, 2.0 Hz, 1H), 7.07 (d, J=2.4 Hz, 1H), 6.74 (d, J=8.4 Hz, 2H), 5.08 (s, 2H), 4.85 (t, J=5.2 Hz, 1H), 4.68 (s, 4H), 4.61 (d, J=5.2 Hz, 2H); ¹³C NMR (100 MHz, CDCl₃, δ): 158.5, 156.5, 148.7, 144.5, 138.4, 136.5, 129.6, 128.71, 128.66, 128.2, 127.6, 127.2, 127.0, 126.7, 126.6, 121.2, 119.4, 112.5, 104.0, 70.6, 54.4, 47.1.

### 5-(Benzyloxy)-N-(4-(cyclohexylmethoxy)benzyl)benzo[d]isothiazol-3-amine, JMM127

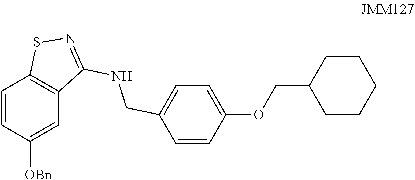 [SMILES: c1ccc(COc2ccc3snc(NCc4ccc(OCC5CCCCC5)cc4)c3c2)cc1]

¹H NMR (400 MHz, CDCl₃, δ): 7.66 (d, J=8.8 Hz, 1H), 7.44-7.34 (m, 7H), 7.22 (dd, J=8.8, 2.4 Hz, 1H), 7.09 (d, J=2.4 Hz, 1H), 6.89 (d, J=8.4 Hz, 2H), 5.08 (s, 2H), 4.92 (t, J=5.2 Hz, 1H), 4.68 (d, J=5.2 Hz, 2H), 3.76 (d, J=6.4 Hz, 2H), 1.90-1.85 (m, 2H), 1.82-1.71 (m, 3H), 1.35-1.18 (m, 4H), 1.10-1.00 (m, 2H); ¹³C NMR (100 MHz, CDCl₃, δ): 158.9, 158.3, 156.5, 144.6, 136.5, 130.7, 129.5, 128.7, 128.2, 127.5, 127.2, 121.2, 119.4, 114.7, 104.0, 73.6, 70.6, 47.0, 37.7, 29.9, 26.5, 25.8.

### 5-(Benzyloxy)-N-(4-(cyclobutylmethoxy)benzyl)benzo[d]isothiazol-3-amine, JMM128

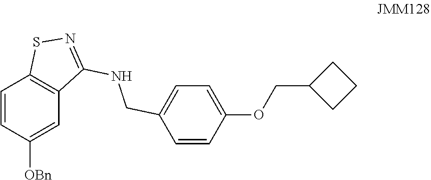 [SMILES: c1ccc(COc2ccc3snc(NCc4ccc(OCC5CCC5)cc4)c3c2)cc1]

¹H NMR (400 MHz, CDCl₃, δ): 7.66 (d, J=8.8 Hz, 1H), 7.45-7.34 (m, 7H), 7.22 (dd, J=8.8, 2.0 Hz, 1H), 7.09 (d, J=2.4 Hz, 1H), 6.90 (d, J=8.4 Hz, 2H), 5.08 (s, 2H), 4.91 (t, J=4.8 Hz, 1H), 4.68 (d, J=4.8 Hz, 2H), 3.93 (d, J=6.8 Hz, 2H), 2.83-2.72 (m, 1H), 2.18-2.11 (m, 2H), 1.99-1.84 (m, 4H); ¹³C NMR (100 MHz, CDCl₃, δ): 158.9, 158.3, 156.5, 144.6, 136.5, 130.8, 129.5, 128.7, 128.2, 127.5, 127.2, 121.2, 119.4, 114.8, 104.0, 72.3, 70.6, 47.0, 34.6, 24.9, 18.6.

### N-(4-(Benzyloxy)benzyl)-5-chlorobenzo[d]isothiazol-3-amine, JMM129

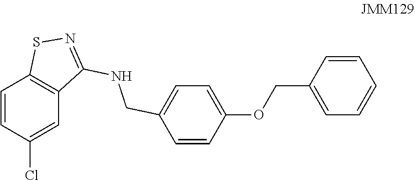 [SMILES: Clc1ccc2snc(NCc3ccc(OCc4ccccc4)cc3)c2c1]

¹H NMR (400 MHz, CDCl₃, δ): 7.70 (d, J=8.8 Hz, 1H), 7.60 (d, J=1.6 Hz, 1H), 7.45-7.33 (m, 8H), 6.97 (d, J=8.8 Hz, 2H), 5.08 (s, 2H), 5.01 (t, J=5.2 Hz, 1H), 4.68 (d, J=5.2 Hz, 2H); ¹³C NMR (100 MHz, CDCl₃, δ): 158.4, 157.8, 150.0, 136.9, 131.0, 130.1, 129.5, 128.6, 128.5, 128.0, 127.5, 127.4, 121.4, 120.8, 115.1, 70.1, 46.9.

### N-(4-(Benzyloxy)benzyl)-5-fluorobenzo[d]isothiazol-3-amine, JMM130

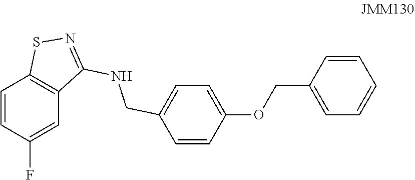 [SMILES: Fc1ccc2snc(NCc3ccc(OCc4ccccc4)cc3)c2c1]

¹H NMR (400 MHz, CDCl₃, δ): 7.71 (ddd, J=8.8, 4.8, 0.4 Hz, 1H), 7.45-7.42 (m, 2H), 7.41-7.33 (m, 5H), 7.29-7.23 (m, 2H), 6.98 (d, J=8.8 Hz, 2H), 5.08 (s, 2H), 4.95 (br s, 1H), 4.68 (d, J=5.2 Hz, 2H); ¹³C NMR (100 MHz, CDCl₃, δ): 160.3 (d, J=242.4 Hz), 158.4, 158.2 (d, J=4.4 Hz), 147.3, 136.9, 131.1, 129.5, 128.6, 128.0, 127.4, 127.2 (d, J=8.0 Hz), 121.6 (d, J=9.2 Hz), 117.3 (d, J=25.3 Hz), 115.1, 106.7 (d, J=23.2 Hz), 70.1, 46.9; ¹⁹F NMR (376 MHz, CDCl₃, δ): −118.7;

### N-(4-(Benzyloxy)benzyl)-5-((4-(trifluoromethyl)benzyl)oxy)benzo[d]isothiazol-3-amine, JMM131

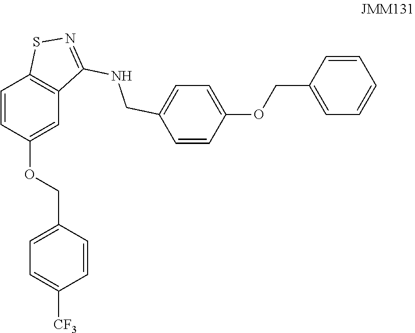 [SMILES: FC(F)(F)c1ccc(COc2ccc3snc(NCc4ccc(OCc5ccccc5)cc4)c3c2)cc1]

¹H NMR (400 MHz, CDCl₃, δ): 7.68 (d, J=8.8 Hz, 1H), 7.65 (d, J=8.4 Hz, 1H), 7.55 (d, J=8.4 Hz, 2H), 7.45-7.33 (m, 8H), 7.22 (dd, J=8.8, 2.0 Hz, 1H), 7.09 (d, J=2.0 Hz, 1H), 6.98 (d, J=8.4 Hz, 2H), 5.14 (s, 2H), 5.08 (s, 2H), 4.94 (t, J=5.2 Hz, 1H), 4.68 (d, J=5.2 Hz, 2H); ¹³C NMR (100 MHz, CDCl₃, δ): 158.3, 158.2, 156.1, 145.0, 140.5, 136.9, 131.3, 130.3 (q, J=32.2 Hz), 129.6, 128.6, 128.0, 127.44, 127.43, 127.2, 125.6 (q, J=3.8 Hz), 124.0 (q, J=270.3 Hz), 121.4, 119.2, 115.1, 104.1, 70.1, 69.7, 46.9;

¹⁹F NMR (376 MHz, CDCl₃, δ): −62.5.

### 5-(Benzyloxy)-N-(4-(thiophen-2-ylmethoxy)benzyl)benzo[d]isothiazol-3-amine, JMM132

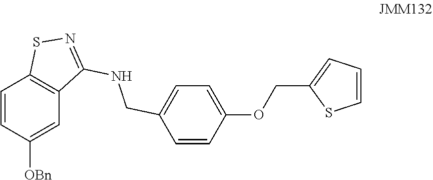 [SMILES: c1ccc(COc2ccc3snc(NCc4ccc(OCc5cccs5)cc4)c3c2)cc1]

¹H NMR (400 MHz, CDCl₃, δ): 7.66 (d, J=8.8 Hz, 1H), 7.45-7.32 (m, 8H), 7.22 (dd, J=8.8, 2.0 Hz, 1H), 7.12-7.10 (m, 2H), 7.01 (dd, J=5.2, 3.6 Hz, 1H), 6.98 (d, J=8.4 Hz, 2H), 5.23 (s, 2H), 5.09 (s, 2H), 4.94 (br s, 1H), 4.69 (d, J=4.0 Hz, 2H); ¹³C NMR (100 MHz, CDCl₃, δ): 158.3, 157.8, 156.5, 144.6, 139.3, 136.5, 131.7, 129.5, 128.7, 128.2, 127.5, 127.2, 126.8, 126.2, 125.3, 121.2, 119.4, 115.2, 104.0, 70.6, 65.1, 46.9.

### N-(Cyclopropylmethyl)-N-(4-(trifluoromethyl)benzyl)benzo[d]isothiazol-3-amine, JMM133

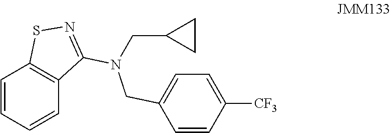 [SMILES: FC(F)(F)c1ccc(CN(CC2CC2)c2nsc3ccccc23)cc1]

Procedure I: NaH (5 eq., 60%) and bromomethyl cyclopropane (5 eq.) were added to a solution of JMM005 (1 eq.) in dry THE at room temperature. The reaction was stirred overnight. The reaction mixture was filtered through Celite and concentrated in vacuo. The crude residue was purified by column chromatography over silica gel (ethyl acetate/hexane). (55%)¹H NMR (400 MHz, CDCl₃, δ): 7.90 (d, J=8.4 Hz, 1H), 7.81 (d, J=8.0 Hz, 1H), 7.60 (d, J=8.4 Hz, 2H), 7.57 (d, J=8.4 Hz, 2H), 7.45 (ddd, J=8.2, 7.2, 1.2 Hz, 1H), 7.30 (ddd, J=8.4, 7.2, 1.2 Hz, 1H), 4.96 (s, 2H), 3.46 (d, J=6.4 Hz, 2H), 1.21-1.13 (m, 1H), 0.53-0.48 (m, 2H), 0.13-0.10 (m, 2H); ¹³C NMR (100 MHz, CDCl₃, δ): 162.7, 153.2, 143.3, 129.4 (d, J=32.1 Hz), 127.72, 127.70, 127.5, 125.4 (q, J=3.8 Hz), 124.2 (q, J=270.2 Hz), 124.0, 123.9, 120.6, 56.0, 54.3, 9.32, 3.78; ¹⁹F NMR (376 MHz, CDCl₃, δ): −62.3.

### N-(4-(Benzyloxy)benzyl)-5-(trifluoromethyl)benzo[d]isothiazol-3-amine, JMM134

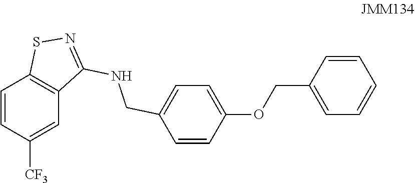 [SMILES: FC(F)(F)c1ccc2snc(NCc3ccc(OCc4ccccc4)cc3)c2c1]

### Procedure B (32%)

¹H NMR (400 MHz, CDCl₃, δ): 7.90-7.88 (m, 2H), 7.69 (dd, J=8.8, 1.2 Hz, 1H), 7.45-7.31 (m, 7H), 6.99 (d, J=8.4 Hz, 2H), 5.16 (br s, 1H), 5.08 (s, 2H), 4.70 (s, 2H); ¹³C NMR (100 MHz, CDCl₃, δ): 158.6, 158.4, 155.0, 136.9, 130.8, 129.5, 128.6, 128.0, 127.4, 126.6 (q, J=32.5 Hz), 126.0, 124.3 (q, J=3.2 Hz), 124.2 (q, J=270.3 Hz), 121.1, 118.6 (q, J=4.2 Hz), 115.2, 70.1, 46.9; ¹⁹F NMR (376 MHz, CDCl₃, δ): −61.5.

### 5-(Benzyloxy)-N-(4-(cyclopropylmethoxy)benzyl)benzo[d]isothiazol-3-amine, JMM135

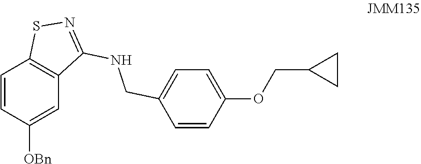 [SMILES: c1ccc(COc2ccc3snc(NCc4ccc(OCC5CC5)cc4)c3c2)cc1]

### Procedure B (34%)

¹H NMR (400 MHz, CDCl₃, δ): 7.66 (d, J=8.8 Hz, 1H), 7.45-7.34 (m, 7H), 7.22 (dd, J=8.8, 2.0 Hz, 1H), 7.09 (d, J=2.0 Hz, 1H), 6.91 (d, J=8.8 Hz, 2H), 5.08 (s, 2H), 4.94 (br s, 1H), 4.68 (s, 2H), 3.80 (d, J=6.8 Hz, 2H), 1.31-1.26 (m, 1H), 0.67-0.63 (m, 2H), 0.37-0.33 (m, 2H); ¹³C NMR (100 MHz, CDCl₃, δ): 158.6, 158.3, 156.5, 144.6, 136.5, 131.0, 129.5, 128.7, 128.2, 127.5, 127.2, 121.2, 119.4, 114.8, 104.0, 72.9, 70.6, 46.9, 10.3, 3.2.

### N-Hexyl-N-(4-(trifluoromethyl)benzyl)benzo[d]isothiazol-3-amine, JMM136

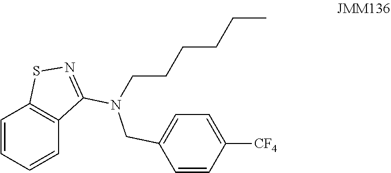

Procedure I: NaH (5 eq., 60%) and 1-iodohexane (5 eq.) were added to a solution of JMM005 (1 eq.) in dry THE at room temperature. The reaction was stirred overnight. The reaction mixture was filtered through Celite and concentrated in vacuo. The crude residue was purified by column chromatography over silica gel (ethyl acetate/hexane). (51%). ¹H NMR (400 MHz, CDCl₃, δ): 7.86 (d, J=8.4 Hz, 1H), 7.80 (d, J=8.0 Hz, 1H), 7.60 (d, J=8.0 Hz, 2H), 7.52 (d, J=8.0 Hz, 2H), 7.45 (ddd, J=8.0, 7.2, 1.2 Hz, 1H), 7.30 (ddd, J=8.4, 6.8, 1.2 Hz, 1H), 4.84 (s, 2H), 3.53 (t, J=8.0 Hz, 2H), 1.77-1.69 (m, 2H), 1.34-1.24 (m, 6H), 0.86 (t, J=6.8 Hz, 3H); ¹³C NMR (100 MHz, CDCl₃, δ): 162.3, 153.2, 143.2, 129.4 (q, J=32.1 Hz), 127.7, 127.6, 127.4, 125.5 (q, J=3.9 Hz), 124.2 (q, J=270.3 Hz), 123.9, 123.8, 120.6, 54.7, 51.8, 31.6, 27.8, 26.7, 22.6, 14.0; ¹⁹F NMR (376 MHz, CDCl₃, δ): −62.4.

### N-(4-(Trifluoromethyl)benzyl)-5-((4-(trifluoromethyl)benzyl)oxy)benzo[d]isothiazol-3-amine, JMM137

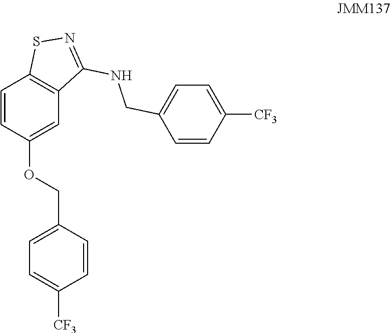 [SMILES: FC(F)(F)c1ccc(CNc2nsc3ccc(OCc4ccc(C(F)(F)F)cc4)cc23)cc1]

### Procedure B (40%)

¹H NMR (400 MHz, CDCl₃, δ): 7.70 (d, J=9.2 Hz, 1H), 7.66 (d, J=8.0 Hz, 2H), 7.60 (d, J=8.4 Hz, 2H), 7.57-7.53 (m, 4H), 7.24 (dd, J=8.8, 2.4 Hz, 1H), 7.13 (d, J=2.0 Hz, 1H), 5.17 (s, 2H), 5.15 (br s, 1H), 4.83 (s, 2H); ¹³C NMR (100 MHz, CDCl₃, δ): 157.9, 156.2, 145.1, 143.2, 140.5, 130.4 (q, J=32.4 Hz), 129.7 (q, J=32.2 Hz), 128.1, 127.4, 127.0, 125.6 (q, J=3.8 Hz), 125.6 (q, J=3.7 Hz), 124.1 (q, J=270.4 Hz), 124.0 (q, J=270.4 Hz), 121.5, 119.2, 104.1, 69.8, 46.8; ¹⁹F NMR (376 MHz, CDCl₃, δ): −62.4, −62.6.

### 5-Chloro-N-(4-(trifluoromethyl)benzyl)benzo[d]isothiazol-3-amine, JMM138

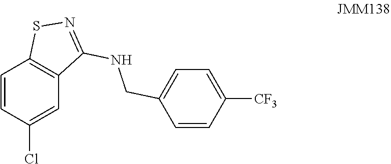 [SMILES: FC(F)(F)c1ccc(CNc2nsc3ccc(Cl)cc23)cc1]

### Procedure B (43%)

¹H NMR (400 MHz, CDCl₃, δ): 7.71 (d, J=8.8 Hz, 1H), 7.65 (d, J=2.0 Hz, 1H), 7.61 (d, J=8.0 Hz, 2H), 7.53 (d, J=8.0 Hz, 2H), 7.45 (dd, J=8.8, 2.0 Hz, 1H), 5.21 (br s, 1H), 4.83 (s, 2H); ¹³C NMR (100 MHz, CDCl₃, δ): 157.4, 150.2, 142.9, 130.3, 129.8 (q, J=32.3 Hz), 128.7, 128.0, 127.3, 125.6 (q, J=3.7 Hz), 124.1 (q, J=270.3 Hz), 121.5, 120.7, 46.7; ¹⁹F NMR (376 MHz, CDCl₃, δ): −62.5.

### 5-Fluoro-N-(4-(trifluoromethyl)benzyl)benzo[d]isothiazol-3-amine, JMM139

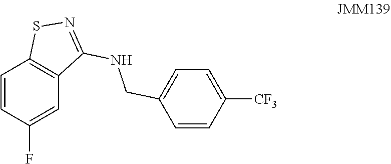 [SMILES: Fc1ccc2snc(NCc3ccc(C(F)(F)F)cc3)c2c1]

### Procedure B (51%)

¹H NMR (400 MHz, CDCl₃, δ): 7.73 (dd, J=8.6, 4.8 Hz, 1H), 7.61 (d, J=8.0 Hz, 2H), 7.53 (d, J=8.0 Hz, 2H), 7.33 (dd, J=8.6, 2.0 Hz, 1H), 7.30-7.25 (m, 1H), 5.17 (br s, 1H), 4.83 (s, 2H); ¹³C NMR (100 MHz, CDCl₃, δ): 160.4 (d, J=242.8 Hz), 157.8 (d, J=4.4 Hz), 147.5, 143.0, 129.8 (q, J=32.1 Hz), 128.0, 127.0 (d, J=8.1 Hz), 125.6 (q, J=3.7 Hz), 124.1 (q, J=270.3 Hz), 121.8 (d, J=9.1 Hz), 117.4 (d, J=25.3 Hz), 106.6 (d, J=23.3 Hz), 46.7; ¹⁹F NMR (376 MHz, CDCl₃, δ): −62.4, −118.4.

### Example 2. Biological Assays

### General Methods

### Cell-Based NPEPPS Activity Assay

HEK293T cells (AATC) were maintained in a complete media containing DMEM (Gibco 11995-065) in the presence of 10% fetal bovine serum (FBS, Gibco 10082147), 50 g/ml penicillin and 50 μg/ml streptomycin (Gibco 15140163) at 37° C. with 5% CO2. NPEPPS activity assay was conducted in a fresh-prepared minimal medium containing essential amino acids, vitamin, AlbuMax (Invitrogen 11021-029) in 1×HBS buffer. Black wall, clear bottom 384-well assay plates (Greiner EK-30092) were coated with poly-L-ornithine (0.1 mg/ml, Sigma P3655) overnight and washed once with 1×PBS. 15 μl minimal medium was then added to each well. 250 nL of compounds dissolved in OmniSolve methylsulfoxide (dimethyl sulfoxide, DMSO, Sigma MX1456P) were pinned to each well via Biomek FX^{P} Liquid Handling Automation (Beckman Coulter) from compound source plates, i.e., either pre-made compound libraries or JMM compounds (20-step, 2-fold dilution) dissolved in OmniSolve DMSO prepared via Biomek automation in 384-well clear V-bottom polypropylene microplates (ThermoScientific 50-823-639). Cells were harvested, resuspended in minimal medium, and seeded at 16,500 cells/15 μl/well in the 384-well assay plates. 24 hrs later, 30 μl 200 μM H-Gln-AMC hydrobromide salt (Q-AMC, Bachem #I-1175) in 1×HBS containing 0.1% DMSO was added into each well. The fluorescent signal at excitation/emission wavelengths of 360/460 nm was measured immediately upon Q-AMC addition and 3 hrs post-Q-AMC. The change in fluorescence is measured as a readout of NPEPPS activity.

### Cell Viability Assay

HEK293T cells (AATC) were maintained in complete media and cell viability assay was conducted in a fresh-prepared minimal medium containing essential amino acids, vitamin, AlbuMax (Invitrogen 11021-029) in 1×HBS buffer. White 384-well assay plates (Greiner EK-30080) were coated with poly-L-ornithine (0.1 mg/ml) overnight and washed once with 1×PBS. 15 μl minimal medium was then added to each well. 250 nl of compounds dissolved in OmniSolve DMSO were pinned to each well via Biomek FX^{P} Liquid Handling Automation from compound source plates. Cells were harvested, resuspended in minimal medium, and seeded at 16,500 cells/15 μl/well in the 384-well assay plates. 24 hrs later, 25 μl/well CellTiter Glo reagent (Promega G7572) was added into each well, and luminescence (0.5 second integration time) was acquired following manufacture's protocol. Luminescence count normalized to DMSO control is used as a readout of cell viability.

### Measurement of Tau Reduction in Mouse Primary Cortical Neurons

Cortical tissue from E15 C57BL/6J mouse embryos was harvested, dissected, and washed in ice-cold HBSS (Invitrogen; 14170-112). Tissue was incubated in 0.25% trypsin (Invitrogen; 15090-046) diluted to 0.05% trypsin in HBSS in the presence of DNase I (Roche; 10104159001) at 37° C. for 10 min. Tissue was washed with cold HBSS and titurated in plating media containing Neurobasal Media (Invitrogen 21103-049), 20% Horse Serum (Invitrogen 26050-088), 25 mM Sucrose, and 0.25% GlutaMax (Invitrogen 35050-061) in the presence of DNase I. Dissociated cells were centrifuged at 125 g for 5 min at 4° C., resuspended in plating media, counted, and plated in poly-L-lysine (Sigma P1274) coated plates at a density of 300,000 cells/mL. Plating media was replaced 16 hrs after plating with feeding media containing Neurobasal Medium supplemented with 1% B27 (Invitrogen 17504-044) and 0.25% GlutaMax. Neurons were cultured for 7 days, followed by treatment with compounds at (5-step dose response, half-log 10 dilution around EC₅₀s from results obtained in the NPEPPS activity assay) for 24 hrs. Cells were then washed once with 1×PBS and 125 μl/well cell lysis buffer (150 mM NaCl, 1 mM EDTA, 50 mM Tris pH 7.4, 1% Triton X-100) supplemented with protease and phosphatase inhibitors (Invitrogen 78440). Following scraping off the plates on ice, lysates were passed through 0.5 CC insulin syringes for 7-8 times on ice and then centrifuged at 13,000 g for 5 minutes at 4° C. Protein concentration in the supernatant transferred to new tubes was measured using Pierce™ BCA Protein Assay Kit (ThermoScientific 23225). Lysates with 5×SDS sample buffer supplemented with β-mercaptoethanol were heated at 95° C. for 5 min in a thermocycler, loaded onto 4-15% mini-PROTEAN TGX Precast gels (BioRad 456-1086) and run at 130V for approx. 1.5 hrs. Samples were transferred to PVDF membranes (BioRad 162-0177), blocked with 5% BSA in TBST for 1 hr at room temperature, probed with total tau primary antibody (Invitrogen AHB0042) overnight at 4° C., washed with TBST, and probed by corresponding HRP-linked secondary antibody incubation (Cell Signaling). Blots were developed with SuperSignal West Pico Chemiluminescent Substrate (ThermoFisher 34579) according to manufacturer's instructions and exposed to an image developer (Biorad ChemiDoc XRS+). Protein band densitometry was measured with ImageLab (Biorad) and normalized to the respective internal control j-Actin band.

Measurement of Tau or Pathological Tau Reduction in Human Tauopathy Patient iPSC-Derived Neurons

iPSCs from patients with the Tau^{P301L} variant (c.C1907T; NCBI NM_001123066, rs63751273, cell line MGH-2046-RC1) or age-matching healthy individuals (cell line 8330-8-RC1) were used. Fibroblasts from each individual were reprogrammed into iPSCs, which were subsequently converted into cortical-enriched neural progenitor cells (NPCs) and differentiated into neuronal cells as previously described^{5,14}. Briefly, cells were cultured in 6-well or 96-well plates coated with poly-L-ornithine (20 μg/ml in water, Sigma P3655) and laminin (5 μg/ml in PBS, Sigma L2020) (POL-coated), in DMEM/F12-B27 medium [70% DMEM (Gibco), 30% Ham's-F12 (Fisher Scientific Corning), 2% B27 (Gibco), 1% penicillin-streptomycin (Gibco)]. Medium was supplemented with EGF (20 ng/ml, Sigma), FGF (20 ng/ml, Stemgent 03-0002) and heparin (5 μg/ml, Sigma H3393), to promote NPC proliferation and expansion. To promote neural differentiation in 96-well plate format, NPCs were plated at approximately 1×10⁵ cells/cm² without growth factors for six to eight weeks, with half medium change two times per week. Compound treatment in 96-well plates was performed in 100 μl medium volume by adding compound directly to each well, followed by incubation at 37° C. for 24 h. Neurons differentiated in 96-well plates were washed in PBS and directly lysed in 75 μl SDS-DTT loading buffer (New England Biolabs). Lysates were transferred to new tubes and boiled for 10 min. Electrophoresis was performed with the Novex NuPAGE SDS-PAGE Gel System (Invitrogen), by running 10 μl of each sample on pre-cast SDS-PAGE. Human recombinant tau protein ladder (Sigma) was used as reference. Gels were transferred onto PVDF membranes (EMD Millipore) using standard procedures. Membranes were blocked in 5% BSA (Sigma A7888) in Tris-buffered saline with 0.1% v/v Tween-20 (TBST) for 2 hrs, incubated overnight with primary antibody for tau TAU5 (Invitrogen AHB0042) or phosphorylated-tau Ser396 (Invitrogen 44752G) at 4° C., followed by corresponding HRP-linked secondary antibody incubation (Cell Signaling). Blots were developed with SuperSignal West Pico Chemiluminescent Substrate (ThermoFisher 34579) according to manufacturer's instructions and exposed to autoradiographic films (LabScientific by ThermoFischer) that, in turn, were scanned on an Epson Perfection V800 Photo Scanner. Protein bands densitometry (pixel mean intensity) was measured with the Adobe Photoshop CS5 Histogram function and normalized to the respective internal control 3-Actin band.

Neuronal Survival Assay in Human Tauopathy Patient iPSC-Derived Neurons

iPSCs derived from patients carrying the Tau^{V337M} variant (MAPT-V337M, line 2B09) or isogenic-corrected control (MAPT-V337V, line 1B06) were differentiated using a Neurogenin 2 (NgN2)-induced neuron protocol modified from previously described protocol^{15,16}. Briefly, iPSCs were seeded at 300,000 cells per well in mTeSR medium (StemCell Technology 85850) supplemented with 1× rock inhibitor (Sigma Y-27632) in poly-L-ornithine (0.01%, Sigma P2636)-coated six-well plates. Cells were transduced with 10 μl/well lentivirus encoding Ngn2::EGFP and 10 μl/well reverse tetracycline-controlled transactivator (rtTA) in mTeSR medium supplemented with 0.4 ug/ml polybrene (Sigma TR-1003) for 12-16 hrs and replenished with fresh mTeSR medium removing lentiviruses. Medium was changed daily for 2-4 days with fresh mTeSR medium. When at 80% confluency, cells were seeded at 5,000 cells per well in mTeSR medium with rock inhibitor on poly-L-ornithine (0.01%) and laminin (10 μg/ml)-coated 96-well plates and switched to a N2 medium containing DMEM/F-12 medium (Gibco 21331020) with 1×N2 supplement (Gibco LS17502048), 1×non-essential amino acids (Gibco 11140050), 1× rock inhibitor, doxycycline (concentration, Sigma D9891), and 20/ng/ml laminin. 24 hrs later, cells were changed to N2 medium with 0.7 ug/ml puromycin (Gibco A1113802) for selection. Two days later, cells were switched in a maturation medium containing DMEM/F-12, 0.5×N2 supplement, 1×B27 (Invitrogen 17504044), glutamax (Invitrogen 35050061), 2% FBS (Gibco 10082147), 10 ng/ml BDNF (StemCell Technology 78005), 10 ng/ml GDNF (StemCell 78058), 10 ng/ml CNTF (StemCell 78010), bFGF (StemCell 78003), 20 ng/ml laminin (Sigma L2020), 7.5 μM RepSox (Selleck S7223) and mouse glia cells. Cells were maintained in the maturation medium by half medium feeding. Survival assay was conducted as previously described⁷. Briefly, neurons were treated with DMSO control, JMM active compounds (e.g., JMM001, JMM013, JMM052, and JMM067), or inactive compound (JMM033) at 1 μM or 5 μM in a maturation medium without RepSox and longitudinally tracked by imaging once every 24-72 hrs for 21 days (Nikon Biostation CT or Molecular Devices ImageExpress). iNs that were no longer detected by EGFP fluorescence were scored as dead. A total of 125 iNs for each experimental groups comparing DMSO and JMM001 or 100 iNs for each experimental groups comparing JMM013, JMM052, or JMM067 with the inactive compound JMM033 were quantified from three biological replicates and were combined into one survival trace in the Kaplan-Meier plots for clarity. The Log-rank (Mantel-Cox) test or the Gehan-Breslow-Wilcoxon (GBW) test was used for statistical analysis.

Target Engagement with Cell-Free and Cellular Thermal Shift Assays

Cell-free thermal shift assay: Recombinant human (rh) NPEPPS protein was diluted in Buffer A (20 mM Tris, 137 mM NaCl, pH 7.5) to a final concentration of 1.25 μM and added to a pre-chilled 384-well microplate (Biorad HSR4801). JMM001, dimethyl sulfoxide (DMSO, Sigma D2650), or NPEPPS inhibitor puromycin dihydrochloride (Sigma P8833) in 1 μl was then added to each well, mixed with gentle aspiration, and incubated with rhNPEPPS for 3 min on ice. SYPRO Orange (Invitrogen S6651) was diluted to 200× concentration in Buffer A and added to each well achieving a final dilution of 1:20, mixed with gentle aspiration. The final assay volume was 20 μl. The plate was sealed followed by a brief spin-down. The samples were analyzed on a Real-time PCR system (Roche LightCycler 480) from 20° C. to 85° C. at a continuous acquisition rate of 5 measurements/° C. and measured at excitation/emission wavelengths of 533/580 nm. Melting temperature is calculated as the lowest derivative of the fluorescence signal as a function of the temperature as previously described^{7,18}.

Cellular thermal shift analysis (CETSA) was conducted at Pelago Bioscience. Briefly, HEK293T cells were harvested and suspended in an experimental Buffer B (20 mM HEPES, 138 mM NaCl, 5 mM KCl, 2 mM CaCl₂), 1 mM MgCl₂, pH7.4) at a density of 4×10⁷ cells/ml. Cell suspension was lysed by three rounds of freeze-thaw cycles with liquid nitrogen and clarified at 20,000 g for 20 min at 4° C. Compounds diluted to 2× final concentration (final solution contains 1% DMSO) in Buffer B were added to equal volume of clarified cell lysates and incubated at 37° C. for 30 min with continuous rotation. 1% DMSO was used as a negative control in parallel. The treated cell lysates were then divided into 60 μl aliquots and subjected to a 12-step heat challenge between 50° C. and 62° C. for 3 min. For the concentration-dependent analyses, clarified cell lysates in 30 aliquots were mixed with an equal volume of compounds or DMSO in Buffer B at 2× intended compound concentration and incubated at 37° C. for 30 min with continuous rotation. JMM001-treated lysates were then heat challenged at 59° C. as determined from its corresponding cellular thermal shift curve. All treated lysates were immediately centrifugated at 20,000 g for 20 min at 4° C. prior to immunoblotting analysis. The immunoblot intensities were obtained by measuring the chemiluminescence counts per square mm (I=count/mm²). The obtained intensities were plotted as the luminescence count normalized to a relevant temperature or control count as specified.

### Animal Subjects

Adult hTau^{P301S} homozygous mice (Tg2541Godt mice, a transgenic mouse model of tauopathy carrying a human Tau^{P301S} mutation¹⁹ and wild-type (WT) littermates on a C57BL/6J background of both genders were used in the in vivo efficacy on Tau histopathology experiments. hTauP301S homozygous mice were obtained as gifts from Dr. Michel Goedert at MRC Laboratory of Molecular Biology, Cambridge, UK and Dr. Stanley Prusiner at UCSF, USA, and were re-derived onto a pure C57BL/6J genetic background by Dr. Prusiner's lab. Mice were periodically outcrossed with WT C57BL/6J mice (Strain #000664) from Jackson Laboratory (Bar Harbor, ME) to maintain genetic diversity. Animals were group-housed in a temperature-controlled facility (73±2° F., 45% humidity, regular 12-hour light/dark cycle, lights on at 7 AM), with food and water ad libitum. All experimental procedures were approved by the ARC administrative office at the University of California Los Angeles (UCLA) and the UCLA institutional animal care and use committee (IACUC) and followed the USDA Animal Welfare Act Regulations, the Public Health Service (PHS) Policy on Humane Care and Use of Laboratory Animals, and the Guide for the Care and Use of Laboratory Animals. Adult male WT mice on a C57BL/6J background (Strain #000664), purchased from Jackson Laboratory (Bar Harbor, ME), were used in the in vivo pharmacokinetics and blood-brain barrier penetration studies. Animals were randomly assigned to experimental groups and tested by experimenters blinded to experimental conditions.

### In Vivo Pharmacokinetics and Blood-Brain Barrier Penetration Studies

In vivo pharmacokinetics and blood-brain barrier penetration studies are conducted at UCSD-DMPK core following their standard of operation protocols. Briefly, freshly prepared JMM compounds (e.g., JMM001, JMM013, JMM015, JMM052, JMM067, and JMM086) formulated in a vehicle (1:1:1:7 DMSO: Cremophor: Ethanol: Saline) were administered to C57BL/6J mice at 20 mg/kg systemically (i.p., i.v., or p.o.) at various time points (0, 0.5, 1, 2, 4, 8, and 24 hrs post-injection) with five mice per group. Plasma samples were collected, and compound plasma levels were measured using LC/MS. For the blood-brain barrier (BBB) penetration studies, compounds (20 mg/kg i.p.) were administered to C57BL/6J mice and brain samples are collected at the time point corresponding where compound showed peak plasma level determined in the plasma PK studies. Brain samples are then processed following and compound concentrations in the brain sample were measured via LC/MS. Compound brain levels are normalized to the mass of brain in a unit of ng/mg.

### Osmotic Pump Surgery

Osmotic minipump (model #2004) were purchased from Alzet (Cupertino, CA, USA) and implanted into mice subcutaneously (s.c.) following the manufacturer's instruction, as previously described²⁰. Briefly, animals are placed on a thermoregulation device and anesthetized using isoflurane [4-5% (induction), 1.5-2.5% (maintenance)]. A sterile drape is used to create a sterile field for surgery after animal preparation. The site between the animal's scapulae is shaved; the exposed skin is cleaned with 70% ethanol, povidone, and 70% ethanol in a sequential order three times. A horizontal incision is performed on the skin between the scapulae for subcutaneous implantation. A deep subcutaneous pocket is made caudal to the incision using blunt dissection. The sterile minipump is inserted into the pocket with the flow moderator facing toward the sacral region of the spinal cord pointing away from the incision. Incisions are then sutured with a wound clipper. Animals are monitored observed continuously during the immediate anesthetic-recovery period until the animal is ambulatory. Animals are monitored daily after anesthetic recovery for 7 days. Wound clips are removed 14 days post surgery.

Assessment of tau histopathological in a mouse model of tauopathy Effects of pharmacological manipulations of JMM compounds on tau histopathology were evaluated via osmotic pump chronic infusion at 20 mg/kg/day s.c. over 28 consecutive days in hTau^{P301S} homozygous mice or WT littermates, starting treatment at the onset of Tau histopathology (i.e., 4.2 months old age). Osmotic pumps (Model #2004, 0.25 μl per hour flow rate) filled with either vehicle [1:1 ratio of DMSO (sigma #D2650): polyethylene glycol 400 (PEG400, Sigma #8170035000)] or JMM compounds (e.g., JMM052, JMM063, or JMM083) were implanted to mice at 4.2 months old for 28 days. Animals are weighted (g) and rectal temperature (° C.) was measured using a thermometer equipped with a mouse rectal probe (Braintree Scientific Inc., Braintree, MA) on treatment day 27. Animals are perfused on treatment day 28 (5 months old age) and immunofluorescence studies on tau histopathology. Animals were perfused with PBS (Gibco #10010-023). Brains were collected and fixed overnight in 4% paraformaldehyde (PFA, Electron Microscopy Sciences #15710). Sagittal sections of 50 m thickness were obtained using a vibratome and blocked with 10% Normal Goat Serum for an hour at room temperature before incubating with primary antibody phospho-Tau AT8 (Ser202, Thr205) (invitrogen #MN1020) overnight at 4° C. The next day, the sections were washed and then incubated with secondary antibody Goat anti-Mouse IgG (H&L)—Alexa Fluor 488 (ThermoFisher #A-11001) for 2 hours at room temperature before being mounted on a glass slide using DAPI Fluoromount (SouthernBiotech #0100-20). The slides were stored at 4° C. until imaging using a confocal microscope. Images were quantified by experimenters blinded from conditions via Fiji (ImageJ) software as previously described²¹. Phospho-Tau AT8 (Ser202, Thr205) antibody (Invitrogen #MN1020) is used to stain tau phosphorylation and cellular pathology in the immunofluorescence experiments.

### Measurement of Photoreceptor Neurodegeneration in a Zebrafish Model of Tauopathy

The rhodopsin::EGFP-MAPT transgenic zebrafish expressing the Tau^{P301L} mutation is generated and maintained as previously described^{22,23}. Tau^{P301L} expression is restricted to the rods of the retina. Photoreceptor image and quantification in central retina sections in the region of the optic nerve head is performed as previously described²²²³.

### Statistical Analyses

The dose-response curves, EC50 and IC50 values were determined using GraphPad Prism Version 9 (CA, USA). GraphPad Prism is also used to generate survival traces in the Kaplan-Meier plots and perform the Log-rank (Mantel-Cox) test or the Gehan-Breslow-Wilcoxon test for the neuronal survival assay in iPSC-derived neurons. Impact of compound effects was analyzed using one-way analysis of variance (ANOVA), corrected for multiple comparisons when necessary, and followed by Bonferroni post hoc tests or t-tests, as appropriate. No gender differences were detected in the Tau cellular histopathology levels (P>0.59) and therefore, results from both genders were pooled for statistical analyses. Statistical analyses were performed using IBM-SPSS Statistics V27.0 (IL, USA) and GraphPad Prism. P<0.05 was considered significant.

### Development of a Cell-Based NPEPPS-Specific Activity Assay

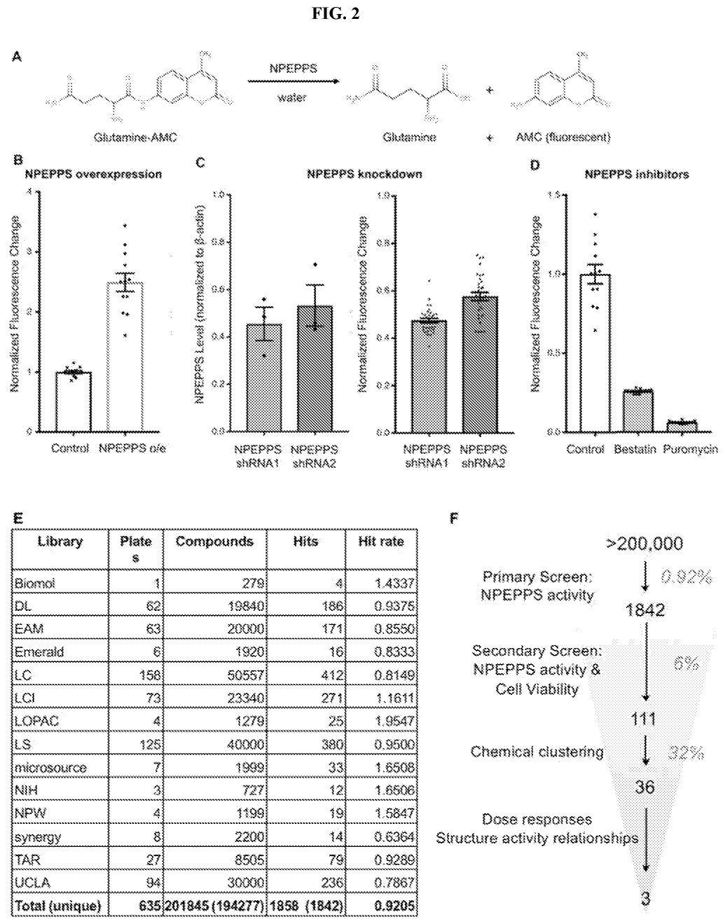

To identify small molecules that either increase NPEPPS levels or NPEPPS activity, a cell-based NPEPPS activity assay amenable to high-throughput screening (HTS) by adapting a fluorescent peptidase activity assay² was developed. Since NPEPPS is one of the only aminopeptidases capable of cleaving glutamine-peptide bonds³ and the major aminopeptidase responsible for degradation of poly-glutamine sequences in the brain⁴, a fluorogenic compound, glutamine-7-amino-4-methylcoumarin (Q-AMC), was selected as a substrate to measure NPEPPS activity. The peptide bond of Q-AMC is hydrolyzed by NPEPPS to release glutamine and a fluorescent product, 7-amino-4-methylcoumarin (AMC), when Q-AMC is added directly to the cell culture media. This change in fluorescence is measured as a readout of aminopeptidase activity (FIG. 2A). Overexpression of NPEPPS in HEK293T cells results in a significant increase in fluorescence, whereas knock-down or chemical inhibition of NPEPPS decrease fluorescence change (FIG. 2B-D), validating that the changes in fluorescence observed in the Q-AMC-based assay directly correlate with changes in NPEPPS activity. A high-throughput (HTS) screen (201,845 compounds) was conducted at the UCLA MSSR core (FIG. 2E). Follow-up assays and chemical clustering identified three series of compounds with distinct chemical clusters (FIG. 2F).

### Overview of the Structure-Activity Relationship Study

The goal of the SAR study was to develop JMM001 analogues of novel structures with high potency (e.g., enhancing NPEPPS activity and reduce tau levels), reduced toxicity, improved solubility, and improved in vivo pharmacokinetics (PK) parameters (e.g., half-life and brain levels). 139 JMM compounds were tested in a series of biological assays, including NPEPPS activity and cell viability in HEK293T cells, tau reduction in mouse primary cortical neurons, the correlation between NPEPPS activity and tau reduction, effects on tau and pathological tau levels and neuronal survival in iPSC-derived neurons from tauopathy patients, in vivo PK and blood-brain barrier (BBB) penetration studies in mice, effects on tau histopathology in mice carrying a hTau^{P301S} mutation, and the role on mitigating neurodegeneration in a zebrafish model expressing a Tau^{P301L} mutation. All analogues are characterized and confirmed by ¹H and ¹³C nuclear magnetic resonance (NMR) spectroscopy for chemical reproducibility and stability prior to biological assays. Target engagement and oral bioavailability of JMM001 are also evaluated. Importantly, the preclinical potency of NPEPPS enhancers on reducing pathological tau levels and rescuing neuronal survival in iPSC-derived neurons from frontotemporal dementia patients harboring disease-causing tau mutations, a human “disease in a dish” model of tauopathy that has been validated previously to predict clinical outcomes of the compounds⁵⁻⁷ was demonstrated. It was also demonstrated that active JMM compounds suppress tau histopathology in a mouse model of tauopathy and a JMM compound mitigates photoreceptor neurodegeneration in a zebrafish model of tauopathy.

### Effects of JMM Compounds on NPEPPS Activity and Cell Viability

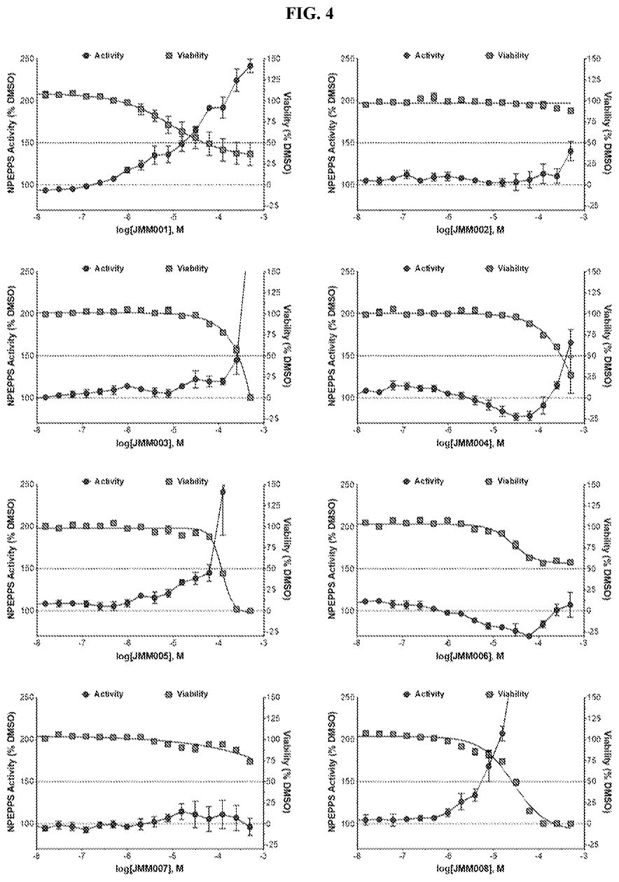

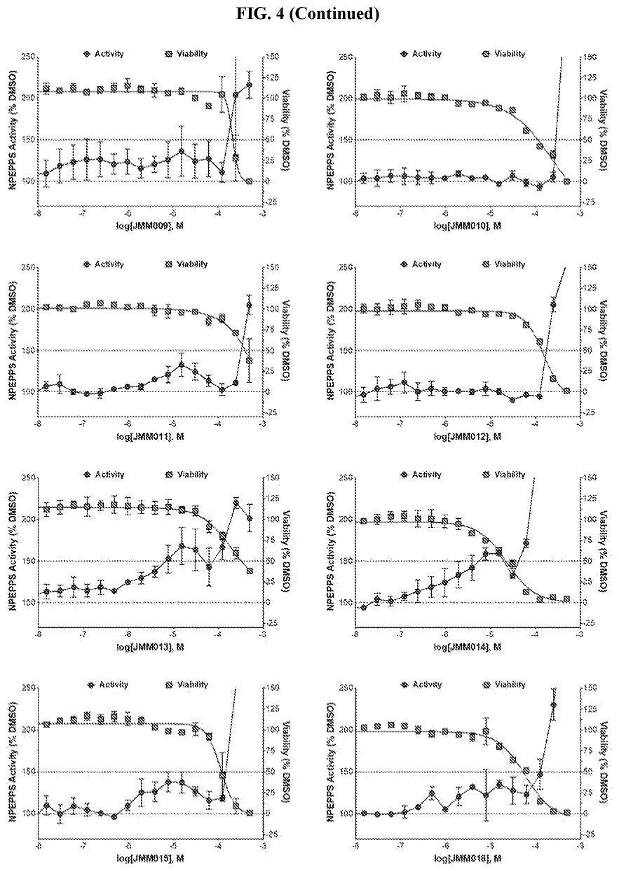

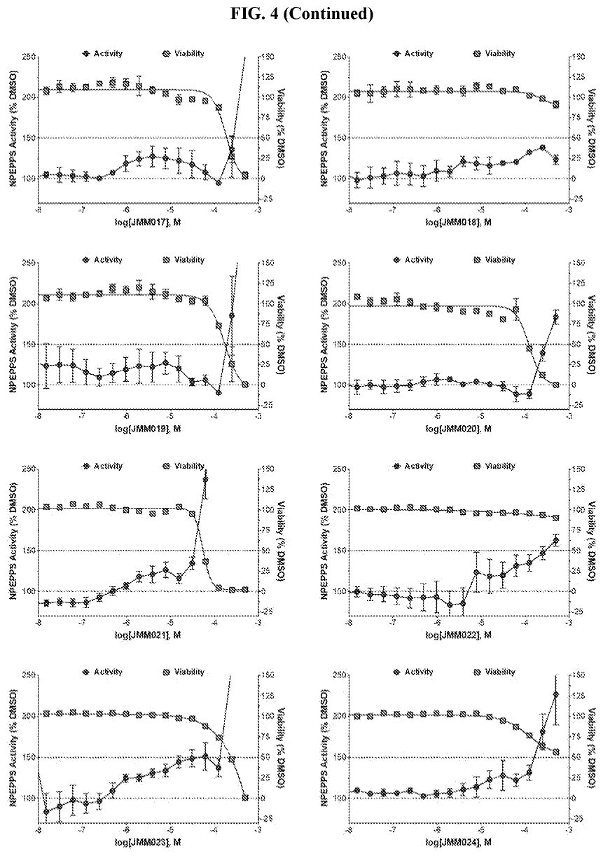

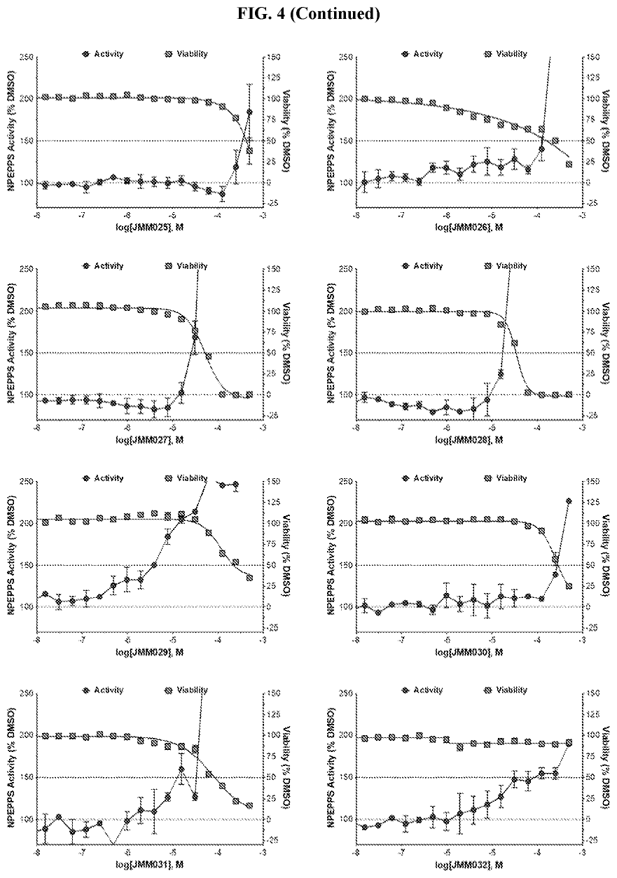

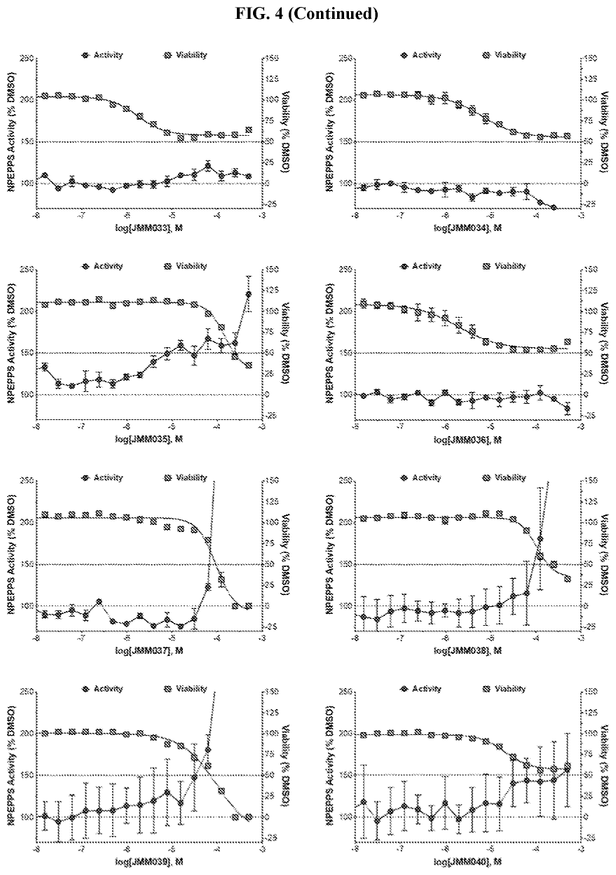

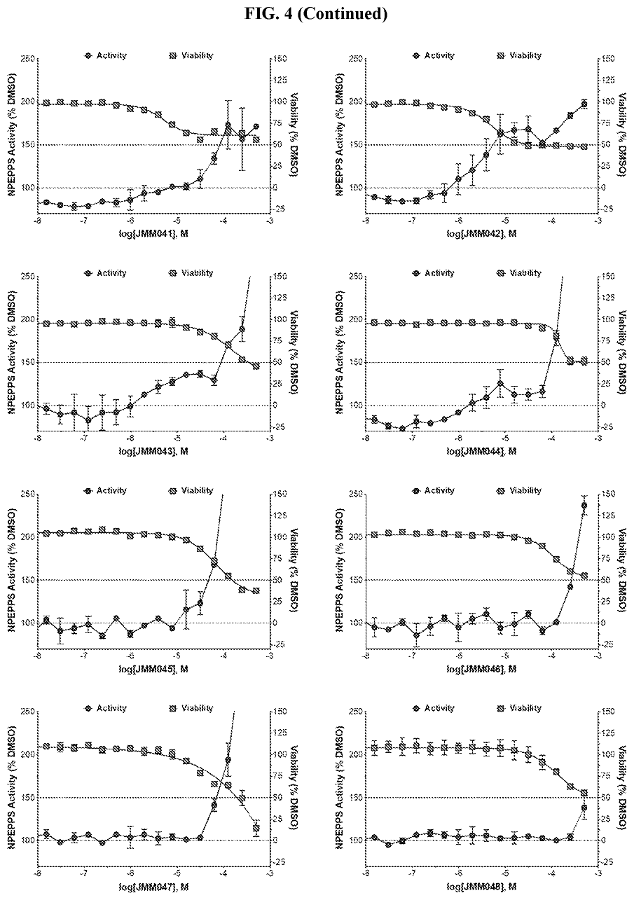

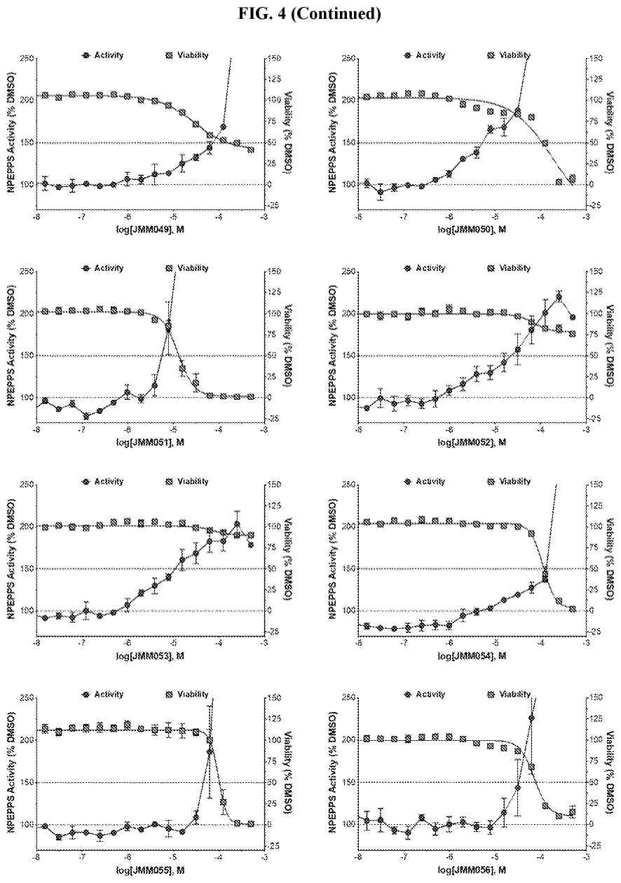

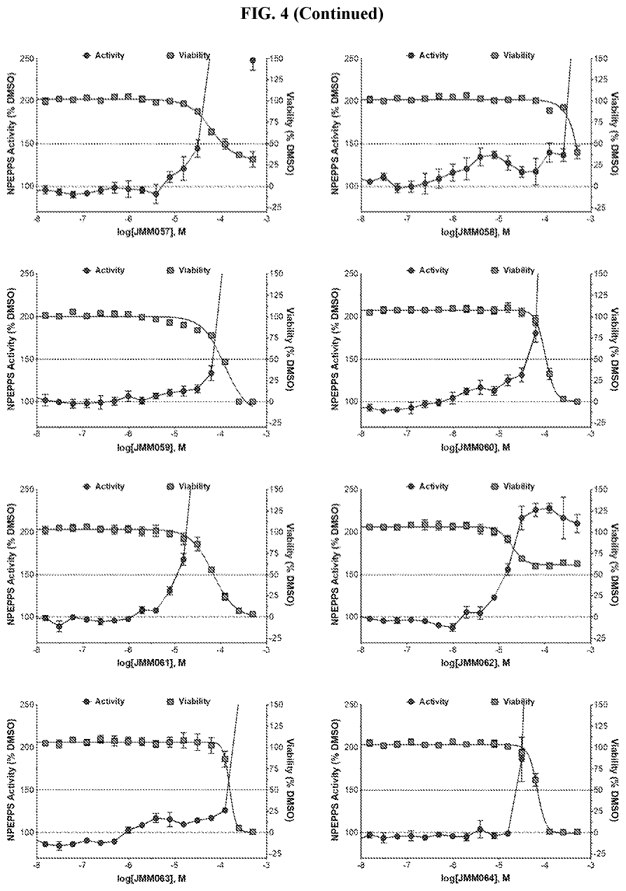

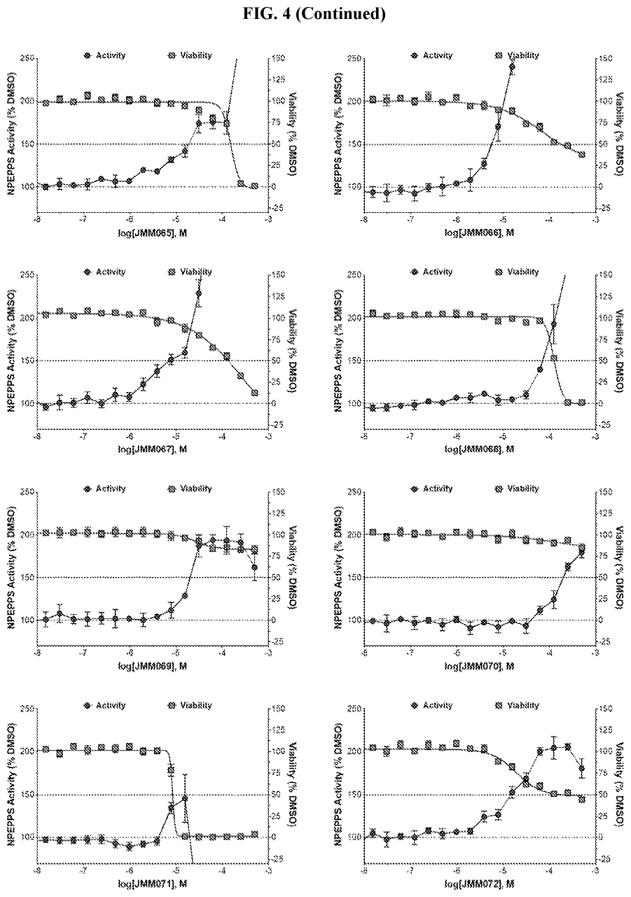

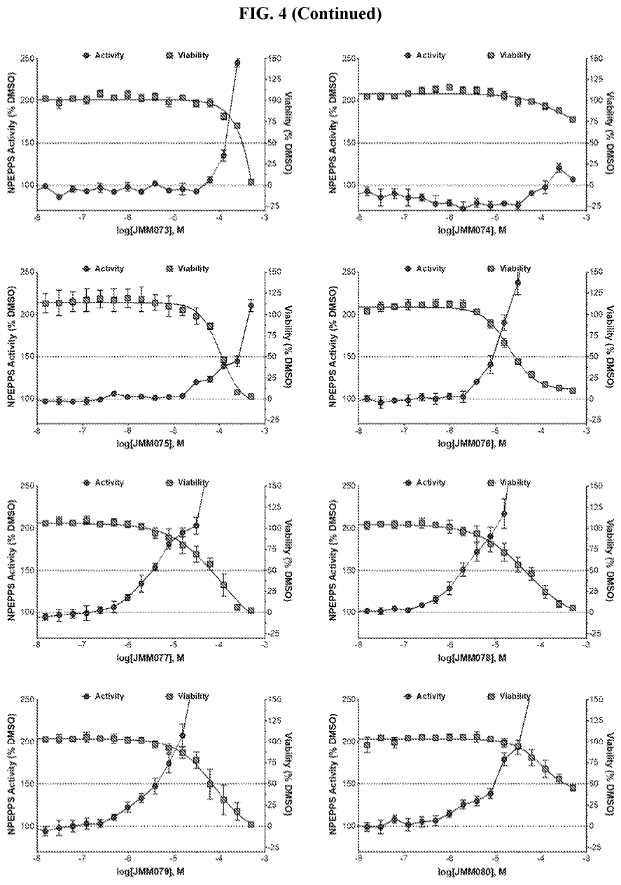

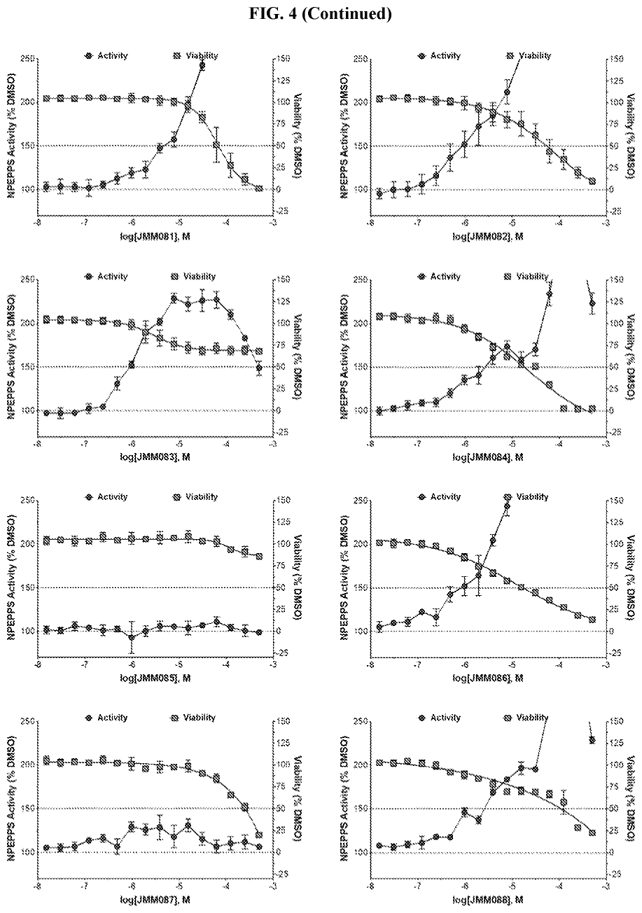

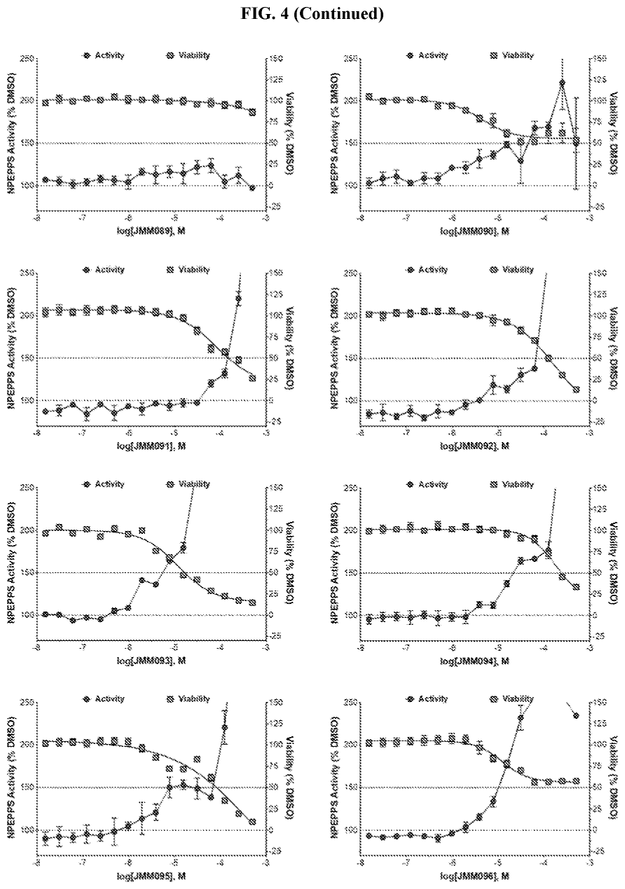

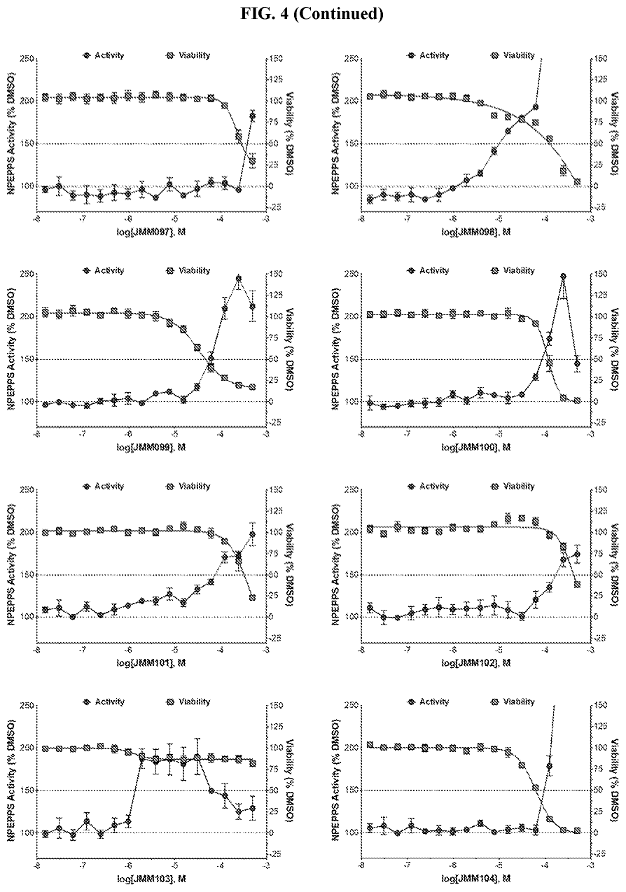

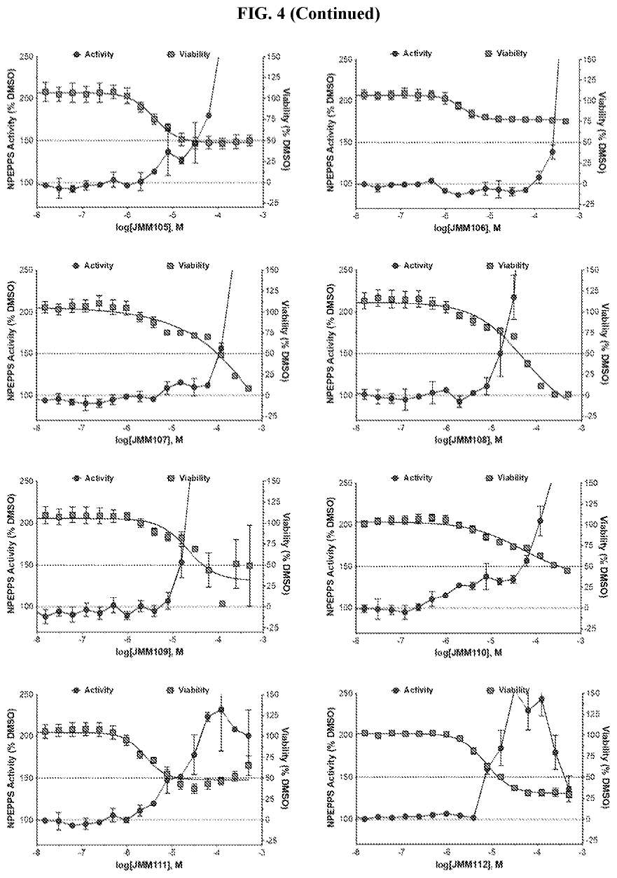

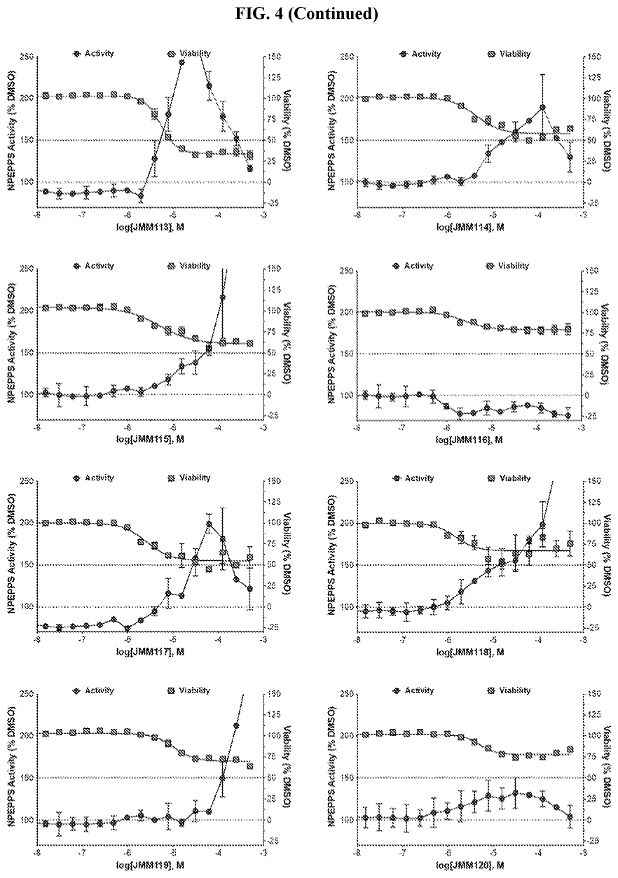

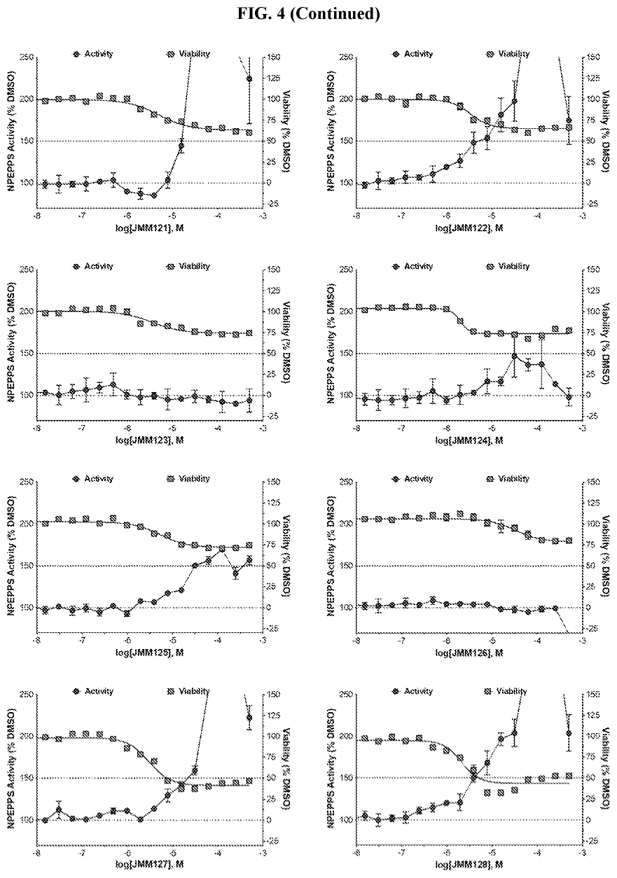

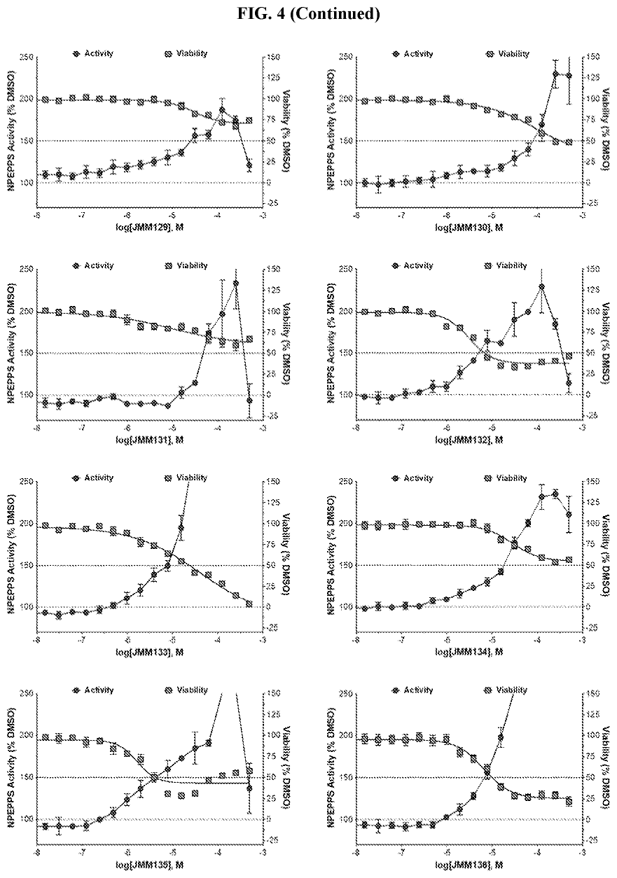

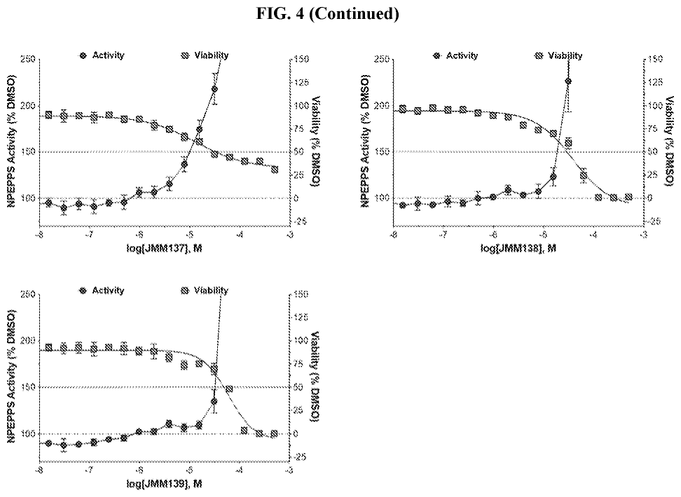

To investigate the effects of JMM compounds that enhance NPEPPS activity and assess their cellular toxicity, 139 compounds in the JMM series (i.e., JMM001-JMM139) were tested in dose responses (20-step, 2-fold dilution) using NPEPPS activity and viability assays in HEK293T cells. 76 compounds harboring different modifications produce concentration-dependent enhancement on NPEPPS activity compared to DMSO control (FIG. 4). Potency as measured by NPEPPS activity indicated by EC50, i.e., the concentration of compound that elevates 50% NPEPPS activity (shows 150% NPEPPS activity normalized to DMSO baseline) and cellular toxicity indicated by IC50, i.e., the concentration that inhibits 50% cell viability, are then calculated. A therapeutic index (TI, a ratio of IC50 over EC50) is used to evaluate the relative safety profiles of the compounds. 31 JMM compounds show single-digit μM or nM potency (EC50<10 μM) and 30 compounds show a therapeutic index greater than 10, major improvements on both potency and safety profiles compared to JMM001 (Table 1). 16 JMM compounds show both improved potency and reduced toxicity (EC50<10 μM and TI>10, Table 1).

TABLE 1
Effects of JMM compounds on NPEPPS activity and cell viability.
Potency on NPEPPS activity is indicated by EC50, i.e., the
concentration of compound that elevates 50% NPEPPS activity
(shows 150% NPEPPS activity normalized to DMSO baseline).
Toxicity is indicated by IC50, i.e., the concentration that
suppresses 50% cell viability. EC50 and IC50 results are
log10 transformed. Relative safety profile is indicated
by therapeutic index (a ratio of IC50 over EC50).

| Compound | log[EC50], M | log[IC50], M | Therapeutic index |
|---|---|---|---|
| JMM001 | −4.80 | −4.19 | 4.05 |
| JMM002 | −2.99 | −2.85 | 1.36 |
| JMM003 | −3.59 | −3.58 | 1.03 |
| JMM004 | −3.39 | −3.43 | 0.90 |
| JMM005 | −4.46 | −3.93 | 3.42 |
| JMM006 | | −3.55 | |
| JMM007 | | −2.76 | |
| JMM008 | −5.43 | −4.58 | 7.09 |
| JMM009 | −3.89 | −3.65 | 1.76 |
| JMM010 | | −4.01 | |
| JMM011 | −3.50 | −3.41 | 1.25 |
| JMM012 | −3.83 | −3.90 | 0.86 |
| JMM013 | −4.82 | −3.39 | 27.36 |
| JMM014 | −4.89 | −4.65 | 1.75 |
| JMM015 | −4.16 | −3.90 | 1.83 |
| JMM016 | −3.90 | −4.32 | 0.38 |
| JMM017 | −3.54 | −3.69 | 0.71 |
| JMM018 | | −2.02 | |
| JMM019 | −3.62 | −3.71 | 0.82 |
| JMM020 | −3.60 | −3.92 | 0.48 |
| JMM021 | −4.49 | −4.23 | 1.80 |
| JMM022 | −3.73 | −2.19 | 34.72 |
| JMM023 | −4.29 | −3.64 | 4.47 |
| JMM024 | −3.80 | −3.36 | 2.76 |
| JMM025 | −3.43 | −3.24 | 1.54 |
| JMM026 | −4.38 | −3.89 | 3.05 |
| JMM027 | −4.27 | −4.28 | 0.98 |
| JMM028 | −4.54 | −4.47 | 1.19 |
| JMM029 | −5.15 | −3.49 | 45.28 |
| JMM030 | −3.66 | −3.59 | 1.16 |
| JMM031 | −5.13 | −4.11 | 10.38 |
| JMM032 | −4.30 | −2.51 | 62.09 |
| JMM033 | | −3.77 | |
| JMM034 | | −3.73 | |
| JMM035 | −4.79 | −3.44 | 22.50 |
| JMM036 | | −3.87 | |
| JMM037 | −3.95 | −4.02 | 0.84 |
| JMM038 | −4.00 | −3.98 | 1.06 |
| JMM039 | −4.69 | −4.21 | 3.03 |
| JMM040 | −3.60 | −3.60 | 1.00 |
| JMM041 | −3.98 | −3.61 | 2.37 |
| JMM042 | −5.24 | −4.23 | 10.41 |
| JMM043 | −4.09 | −3.51 | 3.79 |
| JMM044 | −4.20 | −3.38 | 6.68 |
| JMM045 | −4.27 | −3.73 | 3.49 |
| JMM046 | −3.67 | −3.32 | 2.24 |
| JMM047 | −4.22 | −3.77 | 2.85 |
| JMM048 | −3.28 | −3.25 | 1.09 |
| JMM049 | −4.25 | −3.79 | 2.93 |
| JMM050 | −4.95 | −3.73 | 16.62 |
| JMM051 | −5.15 | −4.85 | 2.03 |
| JMM052 | −4.77 | −2.91 | 72.24 |
| JMM053 | −5.00 | −2.47 | 335.12 |
| JMM054 | −3.91 | −3.88 | 1.08 |
| JMM055 | −4.28 | −3.90 | 2.43 |
| JMM056 | −4.46 | −4.09 | 2.34 |
| JMM057 | −4.50 | −3.82 | 4.85 |
| JMM058 | −3.98 | −3.13 | 7.17 |
| JMM059 | −4.17 | −3.92 | 1.78 |
| JMM060 | −4.27 | −3.99 | 1.88 |
| JMM061 | −4.98 | −4.14 | 6.85 |
| JMM062 | −4.88 | −4.73 | 1.42 |
| JMM063 | −3.83 | −3.71 | 1.33 |
| JMM064 | −4.52 | −4.16 | 2.30 |
| JMM065 | −4.65 | −3.88 | 5.91 |
| JMM066 | −5.19 | −3.76 | 27.03 |
| JMM067 | −5.09 | −3.90 | 15.54 |
| JMM068 | −4.23 | −3.88 | 2.22 |
| JMM069 | −4.75 | −2.82 | 84.99 |
| JMM070 | −3.70 | −2.58 | 13.25 |
| JMM071 | | −5.00 | |
| JMM072 | −4.87 | −3.81 | 11.52 |
| JMM073 | −3.86 | −3.50 | 2.30 |
| JMM074 | | −2.74 | |
| JMM075 | −3.70 | −3.88 | 0.65 |
| JMM076 | −5.01 | −4.70 | 2.03 |
| JMM077 | −5.19 | −4.21 | 9.60 |
| JMM078 | −5.64 | −4.31 | 21.66 |
| JMM079 | −5.45 | −4.16 | 19.15 |
| JMM080 | −5.03 | −3.98 | 11.09 |
| JMM081 | −5.20 | −4.18 | 10.45 |
| JMM082 | −5.58 | −4.12 | 29.32 |
| JMM083 | −6.15 | −2.81 | 2189.48 |
| JMM084 | −5.34 | −4.63 | 5.13 |
| JMM085 | | −2.50 | |
| JMM086 | −5.87 | −4.76 | 13.14 |
| JMM087 | | −3.64 | |
| JMM088 | −6.01 | −4.01 | 100.53 |
| JMM089 | | −2.48 | |
| JMM090 | −4.65 | −4.51 | 1.39 |
| JMM091 | −3.84 | −3.76 | 1.19 |
| JMM092 | −4.35 | −3.90 | 2.84 |
| JMM093 | −5.30 | −4.69 | 4.06 |
| JMM094 | −4.43 | −3.75 | 4.80 |
| JMM095 | −5.11 | −4.14 | 9.25 |
| JMM096 | −4.99 | −4.20 | 6.09 |
| JMM097 | −3.40 | −3.64 | 0.57 |
| JMM098 | −4.73 | −3.97 | 5.68 |
| JMM099 | −4.33 | −4.46 | 0.74 |
| JMM100 | −4.10 | −3.90 | 1.56 |
| JMM101 | −4.15 | −3.39 | 5.74 |
| JMM102 | −3.79 | −3.38 | 2.54 |
| JMM103 | −5.98 | −2.79 | 1543.93 |
| JMM104 | −3.98 | −4.20 | 0.60 |
| JMM105 | −4.58 | −4.27 | 2.02 |
| JMM106 | −3.90 | −3.10 | 6.26 |
| JMM107 | −4.07 | −4.06 | 1.04 |
| JMM108 | −5.25 | −4.39 | 7.13 |
| JMM109 | −5.39 | −4.22 | 14.99 |
| JMM110 | −4.37 | −3.64 | 5.40 |
| JMM111 | −4.99 | −4.56 | 2.74 |
| JMM112 | −5.26 | −4.63 | 4.29 |
| JMM113 | −5.35 | −4.73 | 4.18 |
| JMM114 | −4.58 | −4.20 | 2.42 |
| JMM115 | −4.35 | −3.58 | 5.83 |
| JMM116 | | −3.00 | 0.00 |
| JMM117 | −4.24 | −3.96 | 1.88 |
| JMM118 | −4.59 | −4.81 | 0.60 |
| JMM119 | −3.92 | −3.30 | 4.20 |
| JMM120 | | −2.98 | 0.00 |
| JMM121 | −4.80 | −3.53 | 18.30 |
| JMM122 | −5.33 | −3.49 | 69.07 |
| JMM123 | | −3.18 | 0.00 |
| JMM124 | | −3.14 | 0.00 |
| JMM125 | −4.39 | −3.22 | 14.75 |
| JMM126 | | −2.90 | 0.00 |
| JMM127 | −4.94 | −4.80 | 1.38 |
| JMM128 | −5.35 | −5.02 | 2.12 |
| JMM129 | −4.56 | −3.18 | 24.01 |
| JMM130 | −4.20 | −3.65 | 3.56 |
| JMM131 | −4.21 | −3.45 | 5.78 |
| JMM132 | −5.29 | −4.92 | 2.36 |
| JMM133 | −5.26 | −4.68 | 3.78 |
| JMM134 | −4.86 | −3.59 | 18.60 |
| JMM135 | −5.38 | −5.19 | 1.54 |
| JMM136 | −5.07 | −4.87 | 1.60 |
| JMM137 | −4.93 | −4.41 | 3.30 |
| JMM138 | −4.64 | −4.60 | 1.10 |
| JMM139 | −4.57 | −4.44 | 1.36 |

### Effects of JMM Compounds on Tau Reduction in Mouse Primary Cortical Neurons

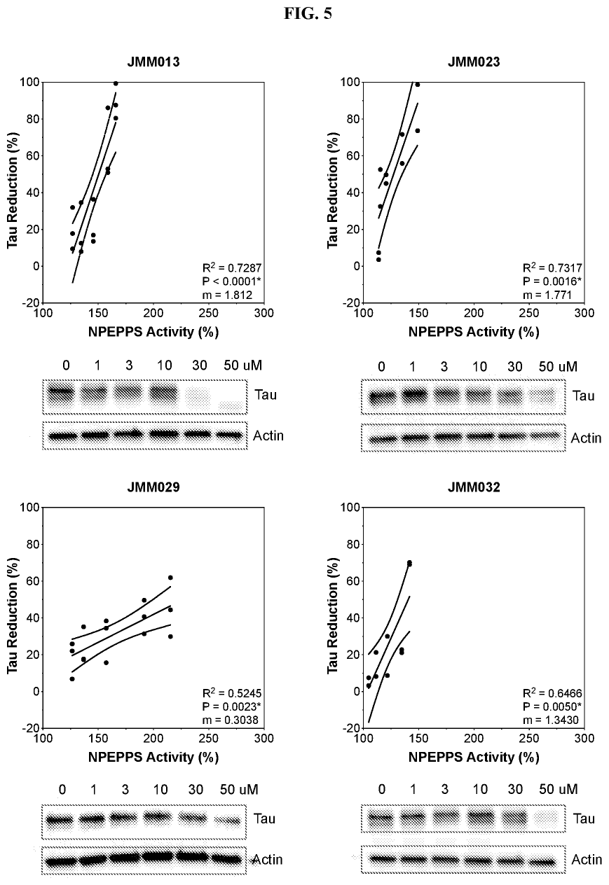

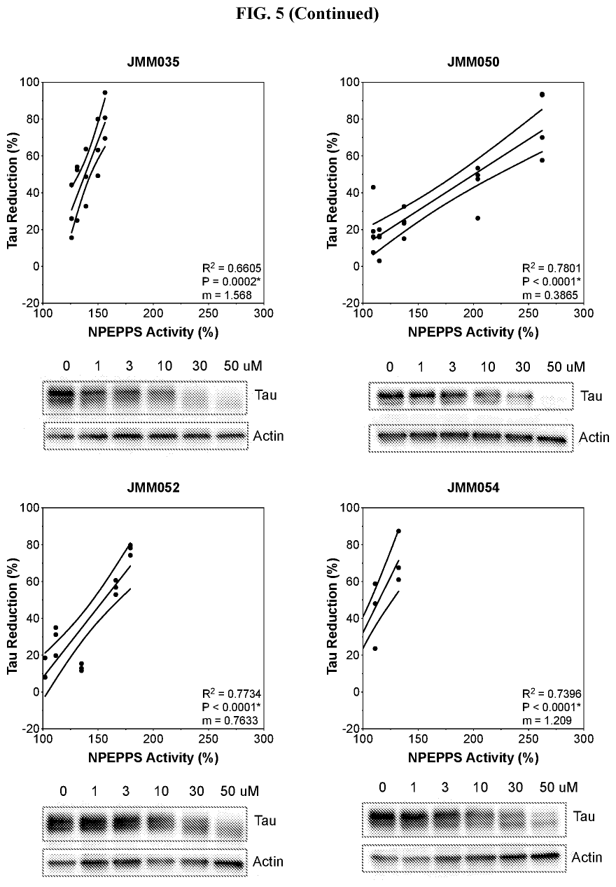

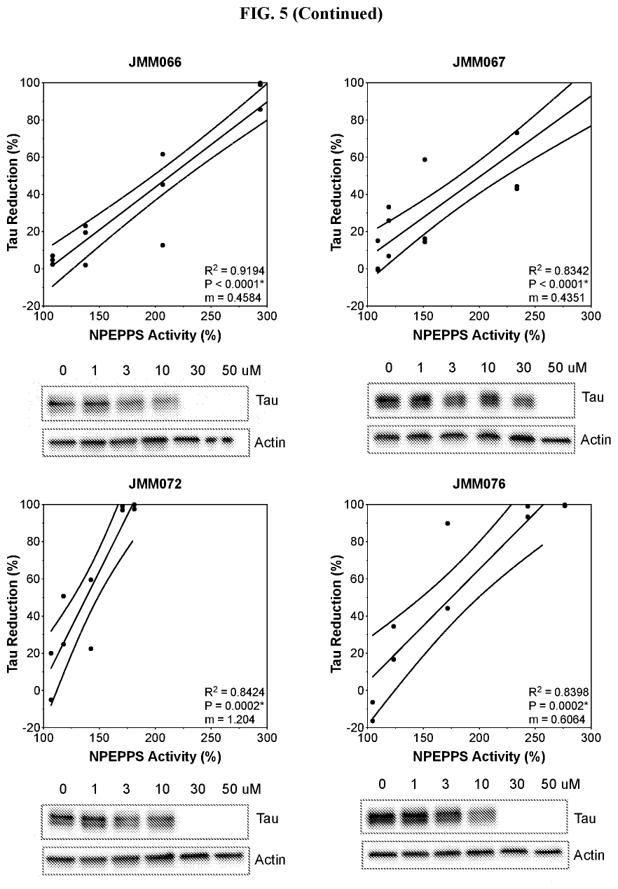

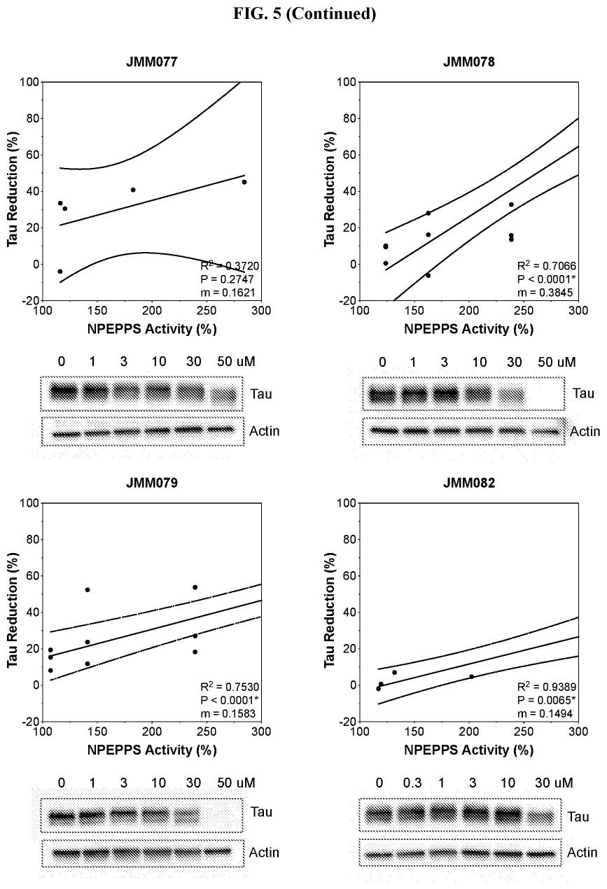

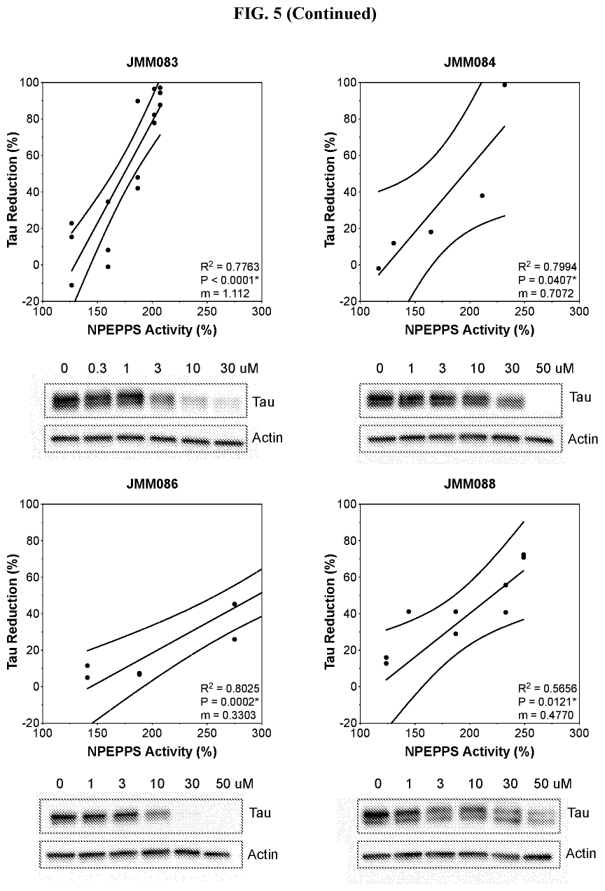

NPEPPS activity has previously been shown to cause reduction of tau protein levels both in cell-free systems and in vivo in mouse and drosophila models of tauopathy,^{1,8,9}. 20 active JMM compounds that showed promising cellular pharmacokinetic responses of NPEPPS activity with minimal toxicity were selected to characterize their effect with respect to tau degradation (5-step dose response, half-log 10 dilution) in mouse primary cortical neurons. All of the active compounds reduce tau levels in a concentration-dependent manner and display positive correlations between NPEPPS activity (fluorescence assay) and tau degradation. 15 of the selected compounds display an R-squared correlation score above 0.7 (FIG. 5). These data therefore demonstrated that active JMM compounds decrease tau levels and their ability to induce tau reduction corresponds directly with their enhancement of NPEPPS activity.

Preclinical Potency: Effects of NPEPPS Enhancers/Activators on Reversing Tau Pathological Phenotypes in Tauopathy Patient iPSC-Derived Neurons

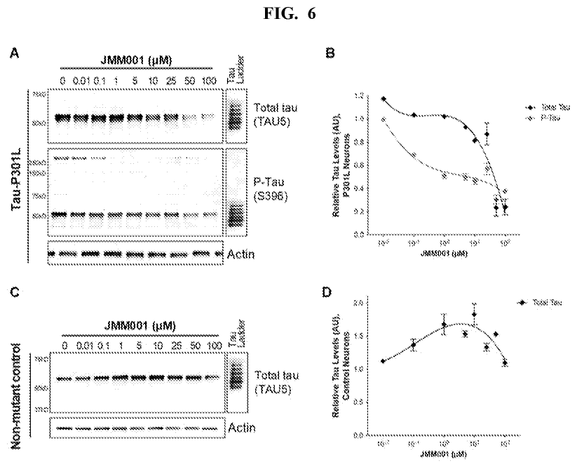

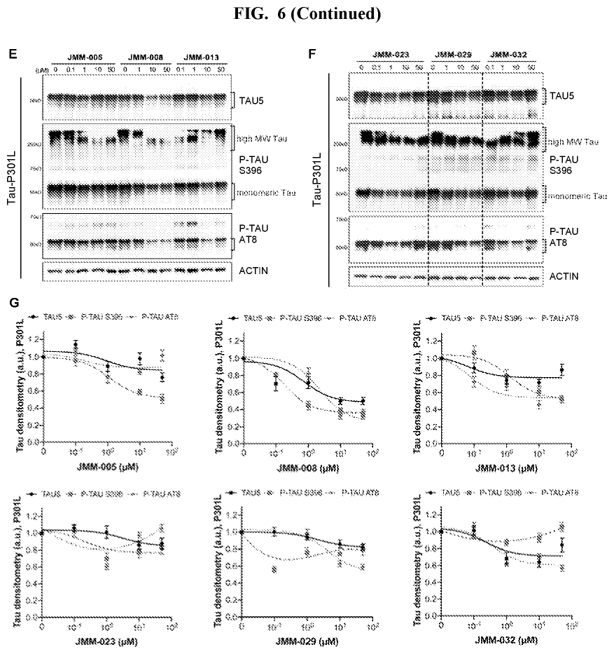

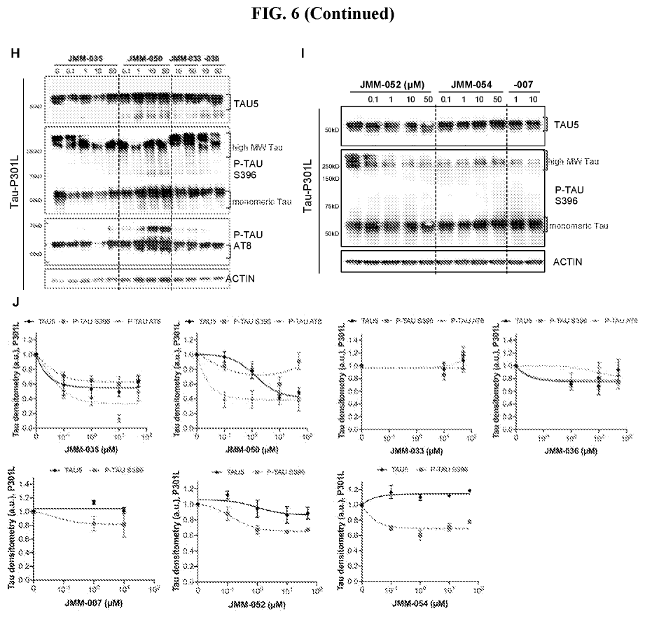

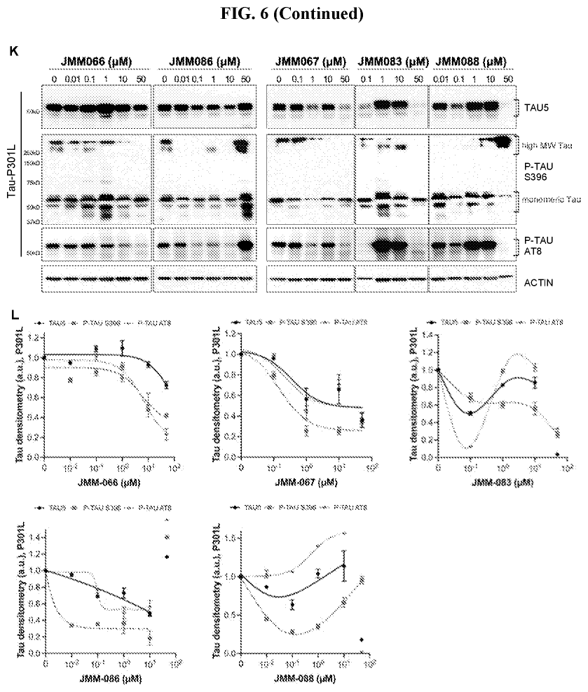

To assess the preclinical potency of the NPEPPS enhancers effect on reversing tau pathological phenotypes in iPSC-derived neurons from patients with primary tauopathy (e.g., Frontotemporal dementia and Progressive supranuclear palsy) with either the TauP^{301L} or Tau^{V337M} mutations, a human “disease in a dish” model of tauopathy validated previously in published work to predict efficacy of tested compounds^{5,6} were studied. This iPSC-derived neuronal model displays critical tau pathological phenotypes, including phosphorylated tau oligomers and low neuronal survival, representing pathological tau aggregation and neuronal loss observed in tauopathy patients, respectively. Active compounds, such as JMM001, JMM008, JMM013, JMM035, JMM050, JMM067, and JMM086, but not inactive analogues such as JMM033, degrade both total tau and pathological phosphorylated tau levels without observable toxicity in iPSC neurons derived from tauopathy patients carrying either the Tau^{A152T} or Tau^{P301L} mutation in a dose-dependent manner (FIGS. 5A-B, 5E-L). JMM001 treatment does not alter tau levels in iPSC neurons derived from age-matching healthy individuals (FIGS. 6C-D).

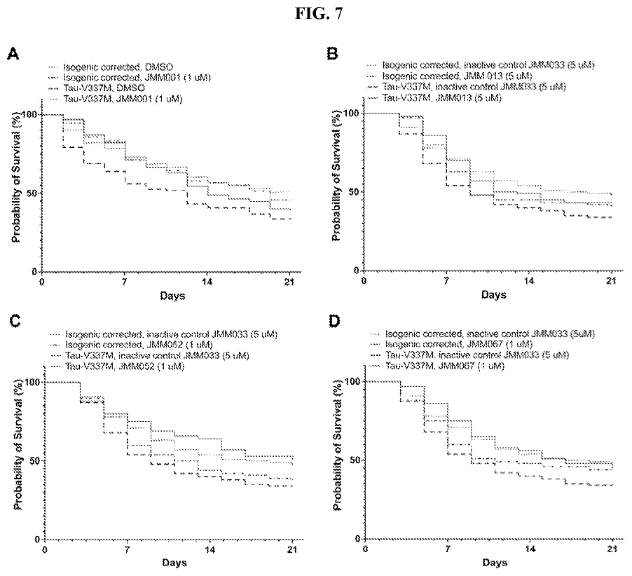

In a longitudinal survival assay, iPSC neurons derived from tauopathy patients carrying the Tau^{V337M} mutation degenerate faster compared to isogenic-corrected (mutation-corrected) controls (FIG. 7). Chronic treatment of JMM001 at 1 μM rescues survival of Tau^{V337M} iPSC neurons compared to DMSO control without affecting isogenic-corrected controls [over 14 days: P<0.05 V337M-JMM001 vs V337M-DMSO; P>0.58 (ns) V337M-JMM001 vs isogenic control-DMSO; over 21 days: p=0.0795 (ns) vs V337M-DMSO, Log-rank (Mantel-Cox) test; FIG. 6A]. Chronic treatment of JMM013 at 5 μM, JMM052 at 1 μM, and JMM067 at 1 μM rescues survival of Tau^{V337M} iPSC neurons compared to inactive analogue JMM033 at 5 μM [over 21 days: JMM013 (Logrank: p=0.060, GBW: p=0.019), JMM052 (Logrank: P<0.01), JMM067 (Logrank: P<0.05) compared to V337M-inactive analogue JMM033] without affecting isogenic-corrected controls [over 21 days: JMM013 (P=0.334), JMM052 (P=0.168), JMM067 (P=0.416) compared to isogenic control-inactive analogue JMM033]. These results obtained from iPSC-derived neurons from patients with dementia and specific tau genetic risk variants validate our strategy of targeting NPEPPS via small molecule JMM series as an effective approach to reduce tau levels and reverse tau-associated pathological phenotypes.

### Direct Target Engagement on NPEPPS

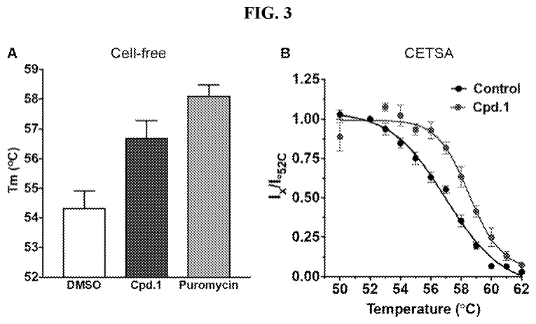

To investigate whether JMM001 binds directly to NPEPPS and engages NPEPPS as its cellular target, both a cell-free protein stability (thermofluor) assay using purified recombinant human NPEPPS (rhNPEPPS) and a cellular thermal shift assay (CETSA¹¹) using Hek293T cell lysates were conducted. JMM001 shows significant, concentration-dependent increases in melting temperature in the cell-free protein stability assay (FIG. 3A), indicating direct binding to NPEPPS. This was confirmed in the CETSA assay, in which JMM001 results in a significant thermal shift of NPEPPS in HEK293T cell lysates in a concentration-dependent manner (FIG. 3B), suggesting that JMM001 engages NPEPPS as its cellular target.

### In Vivo Plasma Pharmacokinetics Profiles of JMM Compounds

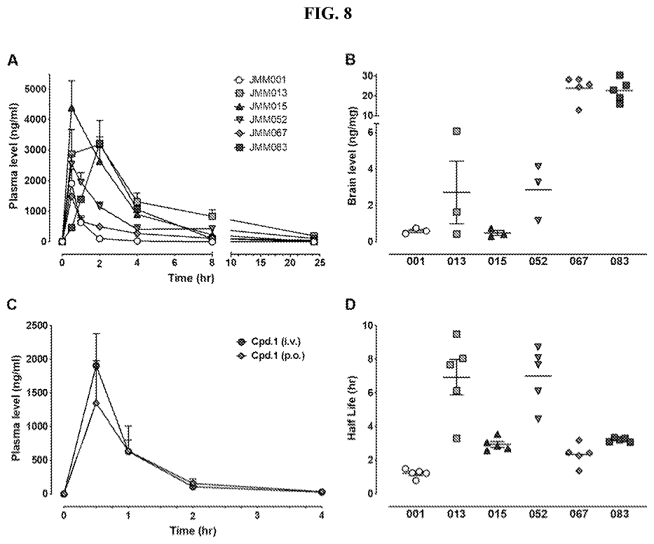

To study the in vivo pharmacokinetic (PK) profiles of JMM compounds, several active JMM compounds with high cellular potency and low toxicity were investigated on plasma PK and blood-brain barrier (BBB) penetration studies conducted in mice at the UCSD Drug Metabolism and Pharmacokinetics (DMPK) core. Blood-brain barrier penetration has been a major challenge in drug discovery for brain disorders. Notably, in vivo PK studies demonstrated that five of the selected JMM compounds cross the blood-brain barrier in mice (FIG. 8B), enabling peripheral delivery to treat neurodegenerative diseases. It was also demonstrated that JMM001 has high oral bioavailability, a preferable feature for clinical applications (FIG. 8C). Most of the selected compounds display improved in vivo plasma PK parameters (e.g., JMM052, JMM067, JMM083), increasing Cmax (maximum plasma concentration observed), half-life, and peak brain concentration compared to JMM001 (FIG. 8A,B,D).

### Preclinical Potency: Effects of NPEPPS Enhancers/Activators on Suppressing Tau Histopathology in a Mouse Model of Tauopathy

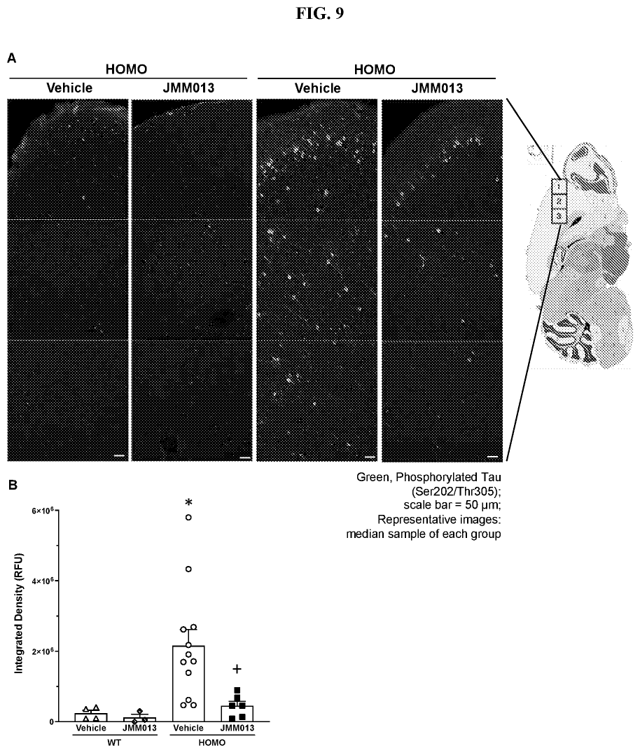

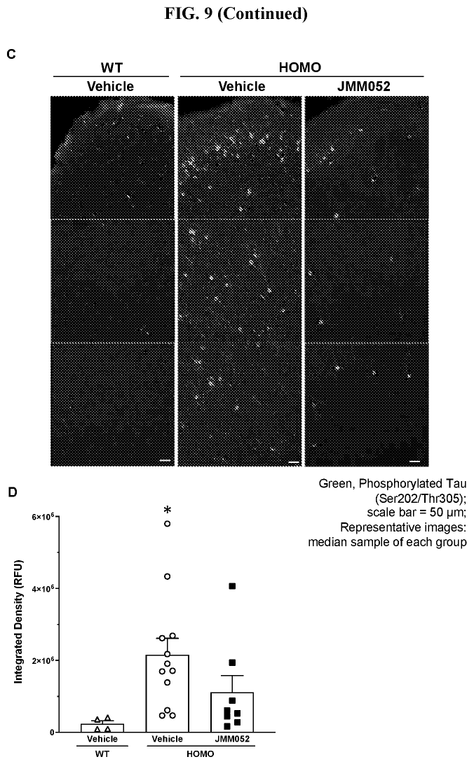

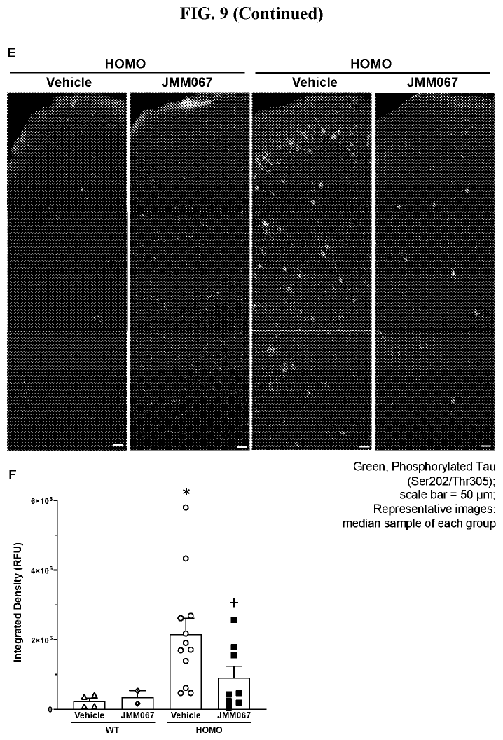

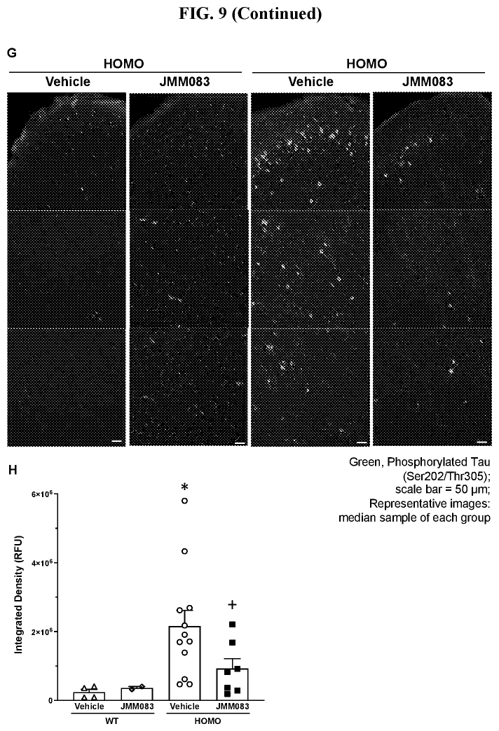

To investigate the therapeutic potential of JMM compounds, preclinical studies using an hTau^{P301S} transgenic mouse model of tauopathy (Allen et al., J Neurosci. 2002) that was comprehensively characterized in our laboratory and confirmed the time course of cellular tau pathology were conducted. Homozygous mice at 4.2 month old (after the onset of Tau histopathology) were treated with active JMM compounds at 20 mg/kg/day s.c. for 28 days. Chronic treatment of JMM013 (FIG. 9A-B), JMM062 (FIG. 9E-F), and JMM083 (FIG. 9G-H) reduced tau histopathology in the cortex of hTau^{P301S} homozygous animals compared to vehicle, demonstrated by reduced positive staining with the phosphorylated tau (Ser202/Thr305) antibody [JMM013: F_{3,21}=5.343, P<0.01, homo-JMM013 vs homo-vehicle (P<0.05); JMM062: F_{3, 22}=3.530, P<0.05, homo-JMM067 vs homo-vehicle (P<0.05); JMM083: F_{3, 21}=3.576, P<0.05, homo-JMM083 vs. homo-vehicle (P<0.05); one-way ANOVA followed by Bonferroni post hoc test or one-tailed t-test, FIG. 9]. Treatment with JMM052 (FIG. 9C-D) does not significantly reduce tau histopathology (F_{2, 21}=3.357, P=0.054, ns; homo-JMM052 vs homo-vehicle (P=0.071, ns). No weight loss or body temperature changes were observed in animals treated with JMM compounds compared to vehicle control.

### Preclinical Potency: Effects of NPEPPS Enhancers/Activators on Mitigating Neurodegeneration in a Zebrafish Model of Tauopathy

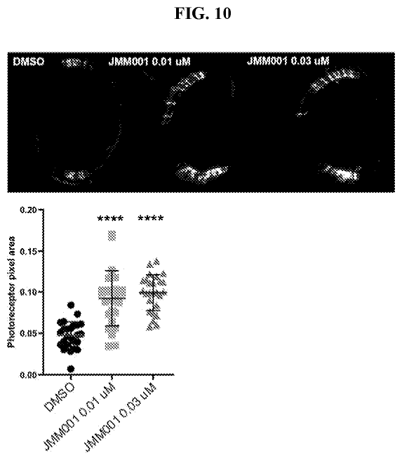

Rhodopsin is a G-protein coupled receptor found in the rod cells of the retina and is a biomarker associated with retinal thinning and degeneration^{22,23}. To further demonstrate the therapeutic potential of NPEPPS enhancers, JMM001 was examined on photoreceptor neurodegeneration in a zebrafish model of tauopathy, in which Tau^{P301L} expression is restricted to the rods of the retina. JMM001 at 0.01 and 0.05 μM rescued the retinal photoreceptor degeneration in the rhodopsin::EGFP-MAPT transgenic zebrafish expressing the TauP^{301L} mutation (P<0.05, FIG. 10).

### Effects of JMM001 on Other Neurodegenerative Diseases

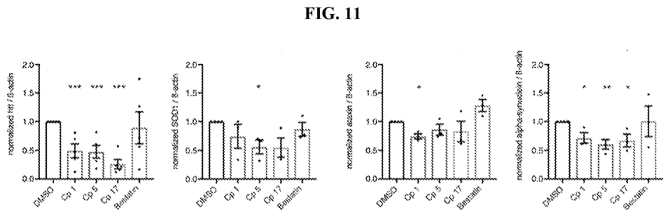

NPEPPS has been shown to be involved in the degradation of other aggregation-prone proteins, especially those containing long poly-glutamine stretches^{4,12,13}. Therefore, whether treatment with JMM001 results in the reduction of other pathogenic proteins in neurodegenerative diseases was tested. JMM001 reduces protein levels of Huntingtin, superoxide dismutase 1 (SOD1), ataxin-3, and α-synuclein in human primary cortical neurons, suggesting potential applications in other neurological disorders associated with protein pathologies, such as Huntington's Disease, Amyotrophic Lateral Sclerosis, Spinocerebral Ataxia, and Parkinson's Disease (FIG. 11).

### REFERENCE

1 Karsten, S. L. et al. A genomic screen for modifiers of tauopathy identifies puromycin-sensitive aminopeptidase as an inhibitor of tau-induced neurodegeneration. Neuron 51, 549-560, doi:10.1016/j.neuron.2006.07.019 (2006).
2 Zimmerman, M., Ashe, B., Yurewicz, E. C. & Patel, G. Sensitive assays for trypsin, elastase, and chymotrypsin using new fluorogenic substrates. Anal Biochem 78, 47-51, doi:10.1016/0003-2697(77)90006-9 (1977).
3 Constam, D. B. et al. Puromycin-sensitive aminopeptidase. Sequence analysis, expression, and functional characterization. J Biol Chem 270, 26931-26939 (1995).
4 Bhutani, N., Venkatraman, P. & Goldberg, A. L. Puromycin-sensitive aminopeptidase is the major peptidase responsible for digesting polyglutamine sequences released by proteasomes during protein degradation. EMBO J 26, 1385-1396, doi:10.1038/sj.emboj.7601592 (2007).
Silva, M. C. et al. Human iPSC-Derived Neuronal Model of Tau-A152T Frontotemporal Dementia Reveals Tau-Mediated Mechanisms of Neuronal Vulnerability. Stem Cell Reports 7, 325-340, doi:10.1016/j.stemcr.2016.08.001 (2016).
6 Swarup, V. et al. Identification of evolutionarily conserved gene networks mediating neurodegenerative dementia. Nat Med 25, 152-164, doi:10.1038/s41591-018-0223-3 (2019).
7 Shi, Y. et al. Haploinsufficiency leads to neurodegeneration in C90RF72 ALS/FTD human induced motor neurons. Nat Med 24, 313-325, doi:10.1038/nm.4490 (2018).
8 Kudo, L. C. et al. Puromycin-sensitive aminopeptidase (PSA/NPEPPS) impedes development of neuropathology in hPSA/TAU(P301L) double-transgenic mice. Hum Mol Genet 20, 1820-1833, doi:10.1093/hmg/ddr065 (2011).
9 Sengupta, S. et al. Degradation of tau protein by puromycin-sensitive aminopeptidase in vitro. Biochemistry 45, 15111-15119, doi:10.1021/bi061830d (2006).
10 Stein, J. L. et al. A quantitative framework to evaluate modeling of cortical development by neural stem cells. Neuron 83, 69-86, doi:10.1016/j.neuron.2014.05.035 (2014).
11 Martinez Molina, D. & Nordlund, P. The Cellular Thermal Shift Assay: A Novel Biophysical Assay for In Situ Drug Target Engagement and Mechanistic Biomarker Studies. Annu Rev Pharmacol Toxicol 56, 141-161, doi:10.1146/annurev-pharmtox-010715-103715 (2016).
12 Menzies, F. M. et al. Puromycin-sensitive aminopeptidase protects against aggregation-prone proteins via autophagy. Hum Mol Genet 19, 4573-4586, doi:10.1093/hmg/ddq385 (2010).
13 Ren, G. et al. Cu, Zn-superoxide dismutase 1 (SOD1) is a novel target of Puromycin-sensitive aminopeptidase (PSA/NPEPPS): PSA/NPEPPS is a possible modifier of amyotrophic lateral sclerosis. Mol Neurodegener 6, 29, doi:10.1186/1750-1326-6-29 (2011).
14 Almeida, S. et al. Induced pluripotent stem cell models of progranulin-deficient frontotemporal dementia uncover specific reversible neuronal defects. Cell Rep 2, 789-798, doi:10.1016/j.celrep.2012.09.007 (2012).
15 Zhang, Y. et al. Rapid single-step induction of functional neurons from human pluripotent stem cells. Neuron 78, 785-798, doi:10.1016/j.neuron.2013.05.029 (2013).
16 Busskamp, V. et al. Rapid neurogenesis through transcriptional activation in human stem cells. Mol Syst Biol 10, 760, doi:10.15252/msb.20145508 (2014).
17 Lavinder, J. J., Hari, S. B., Sullivan, B. J. & Magliery, T. J. High-throughput thermal scanning: a general, rapid dye-binding thermal shift screen for protein engineering. J Am Chem Soc 131, 3794-3795, doi:10.1021/ja8049063 (2009).
18 Ericsson, U. B., Hallberg, B. M., Detitta, G. T., Dekker, N. & Nordlund, P. Thermofluor-based high-throughput stability optimization of proteins for structural studies. Anal Biochem 357, 289-298, doi:10.1016/j.ab.2006.07.027 (2006).
19 Allen, B. et al. Abundant tau filaments and nonapoptotic neurodegeneration in transgenic mice expressing human P301S tau protein. J Neurosci 22(21), 9340-9351, doi: 10.1523/JNEUROSCI.22-21-09340.2002 (2002).
20 Deng, L. et al. Prophylactic treatment with the tricyclic antidepressant desipramine prevents development of paclitaxel-induced neuropathic pain through activation of endogenous analgesic systems. Pharmacological research 114, 75-89. doi:10.1016/j.phrs.2016.10.007 (2016).
21 Xu, N. et al. Spared Nerve Injury Increases the Expression of Microglia M1 Markers in the Prefrontal Cortex of Rats and Provokes Depression-Like Behaviors. Frontiers in neuroscience 11, 209. doi:10.3389/fnins.2017.00209 (2017).
22 Lopez, A. et al. A152T tau allele causes neurodegeneration that can be ameliorated in a zebrafish model by autophagy induction. Brain: a journal of neurology, 140(4), 1128-1146. doi:10.1093/brain/awx005 (2017).
23 Moreau, K. et al. PICALM modulates autophagy activity and tau accumulation. Nat Commun 5, 4998. doi:10.1038/ncomms5998 (2014).

### CHEMICAL SYNTHESIS REFERENCES

[1] F. Bennett, et al. PCT Int. Appl., 2009032123, 12 Mar. 2009.
[2] A. Martinez-Asencio, D. J. Ramon, M. Yus, Tetrahedron 2011, 67, 3140-3149.
[3] X.-W. Chen, Y.-Y. Sun, L. Fu, J.-Q. Li, Eur. J. Med. Chem. 2016, 123, 332-353.
[4] H. Rauer, U.S. Pat. Appl. Publ. 20040009973, 15 Jan. 2004.
[5] T. Adler, J. Bonjoch, J. Clayden, M. Font-Bardia, M. Pickworth, X. Solans, D. Sole, L. Vallverdú, Org. Biomol. Chem. 2005, 3, 3173-3183.
[6] S. Murata, T. Sugawara, H. Iwamura, J. Am. Chem. Soc. 1985, 107, 6317-6329.
[7] S. Roy, S. K. Das, B. Chattopadhyay, Angew. Chem. Int. Ed. 2018, 57, 2238-2243.
[8] S. Schneider, PCT Int. Appl., 2011092197, 4 Aug. 2011.
[9] K. Ko, H.-J. Kim, P.-S. Ho, S. O. Lee, J.-E. Lee, C.-R. Min, Y. C. Kim, J.-H. Yoon, E.-J. Park, Y.-J. Kwon, J.-H. Yun, D.-O. Yoon, J.-S. Kim, W.-S. Park, S.-S. Oh, Y.-M. Song, W.-K. Cho, K. Morikawa, K.-J. Lee, C.-H. Park, J. Med. Chem. 2018, 61, 2949-2961.
[10] S. A. Boyd, PCT Int. Appl., 2005095391, 13 Oct. 2005.
[11] K. E. Torraca, X. Huang, C. A. Parrish, S. L. Buchwald, J. Am. Chem. Soc. 2001, 123, 10770-10771.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound of formula I: 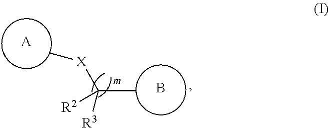
or a pharmaceutically acceptable salt thereof, wherein:
X is C(R⁰)₂, NR¹, O or S;
ring A is C₆₋₁₀ aryl or 5- to 10-membered heteroaryl;
ring B is C₆₋₁₄ aryl or 5- to 10-membered heteroaryl;
R⁰ is halogen, amino, hydroxyl, alkoxy, cyano, nitro, alkyl, alkenyl, alkynyl, carbocyclyl, heterocyclyl, aryl, or heteroaryl;
R¹ is hydrogen, sulfonyl, alkyl, aralkyl, alkenyl, alkynyl, carbocyclyl, heterocyclyl, aryl, or heteroaryl;
R² and R³ independently are hydrogen, alkyl, alkenyl, alkynyl, carbocyclyl, heterocyclyl, aryl, or heteroaryl; or
R², R³, and the carbon atom to which they are connected, complete an oxo group (C═O); or
R³, ring B, and the intervening atoms, complete a carbocyclyl, heterocyclyl, aryl, or heteroaryl;
m is 0, 1, or 2, preferably 1, and
provided the compound is not

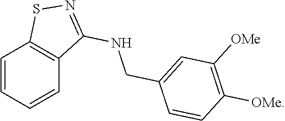 [SMILES: COc1ccc(CNc2nsc3ccccc23)cc1OC]

2. The compound of claim 1, wherein the compound is of formula I: 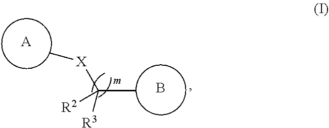
or a pharmaceutically acceptable salt thereof, wherein:
X is C(R⁰)₂, NR¹, O or S;
ring A is C₆₋₁₀ aryl or 6- to 10-membered heteroaryl;
ring B is C₆₋₁₄ aryl or 6- to 10-membered heteroaryl;
R⁰ is halogen, amino, hydroxyl, alkoxy, cyano, nitro, alkyl, alkenyl, alkynyl, carbocyclyl, heterocyclyl, aryl, or heteroaryl;
R¹ is hydrogen, sulfonyl, alkyl, aralkyl, alkenyl, alkynyl, carbocyclyl, heterocyclyl, aryl, or heteroaryl;
R² and R³ independently are hydrogen, alkyl, alkenyl, alkynyl, carbocyclyl, heterocyclyl, aryl, or heteroaryl; or
R², R³, and the carbon atom to which they are connected, complete an oxo group (C═O); or
R³, ring B, and the intervening atoms, complete a carbocyclyl, heterocyclyl, aryl, or heteroaryl;
m is 0, 1, or 2, preferably 1.

3. The compound of claim 1 or 2, wherein X is NR¹.

4. The compound of any one of claims 1-3, wherein A is 5- to 10-membered heteroaryl.

5. The compound of claim 4, wherein A is 6-membered heteroaryl.

6. The compound of claim 5, wherein A is selected from optionally substituted

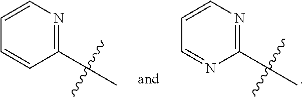 [SMILES: *c1ccccn1 ; *c1ncccn1]

7. The compound of claim 4, wherein A is a 9- to 10-membered heteroaryl.

8. The compound of claim 6, wherein A is selected from optionally substituted

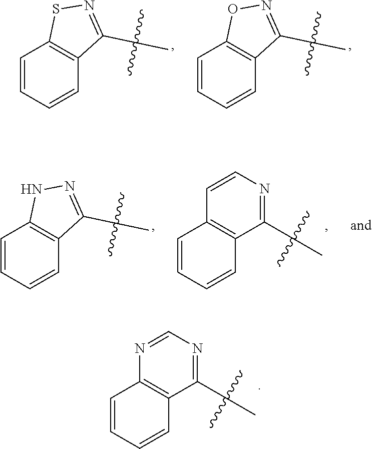 [SMILES: *c1nsc2ccccc12 ; *c1noc2ccccc12 ; *c1n[nH]c2ccccc12 ; *c1nccc2ccccc12 ; *c1ncnc2ccccc12]

9. The compound of claim 7, wherein A is optionally substituted

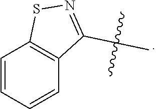 [SMILES: *c1nsc2ccccc12]

10. The compound of any one of claims 1-9, wherein A is optionally substituted with one or more of bromo, chloro, fluoro, ethynyl, cyano, benzyloxy, trifluoromethyl, and methoxy.

11. The compound of any one of claims 1-9, wherein A is unsubstituted.

12. The compound of any one of claims 1-11, wherein B is C₆₋₁₄ aryl.

13. The compound of claim 12, wherein B is selected from optionally substituted

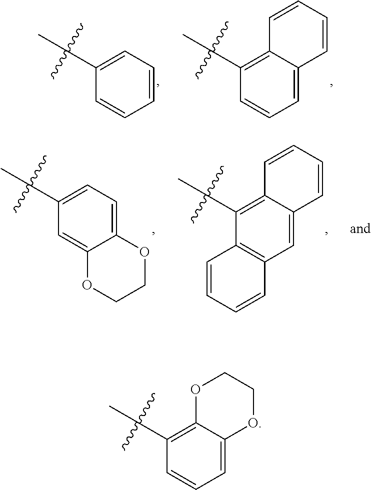

14. The compound of claim 13, wherein B is optionally substituted

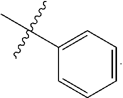 [SMILES: *c1ccccc1]

15. The compound of any one of claims 1-11, wherein B is 6- to 10-membered heteroaryl.

16. The compound of claim 15, wherein B is selected from optionally substituted

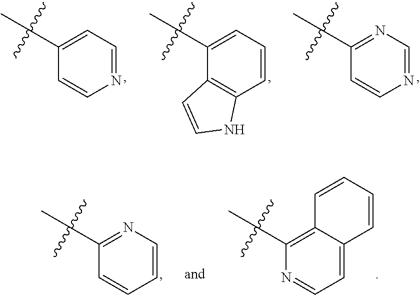 [SMILES: *c1ccncc1 ; *c1cccc2[nH]ccc12 ; *c1ccncn1 ; *c1ccccn1 ; *c1nccc2ccccc12]

17. The compound of any one of claims 12-16, wherein B is optionally substituted with one or more of phenyl, benzyl, methyl, —CH₂—O-phenyl, trifluoromethyl, fluoro, chloro, bromo, trifluoromethoxy, —CO₂Me, cyano, nitro, difluoromethyl, —SCF₃, —OR⁶, —NHR⁶, or —N(R⁶)₂, wherein each R⁶ is independently hydrogen, alkyl, aryl, or heteroaryl.

18. The compound of any one of claims 12-16, wherein B is unsubstituted.

19. The compound of claim 1 or 2, wherein the compound of formula I is a compound of formula I-1 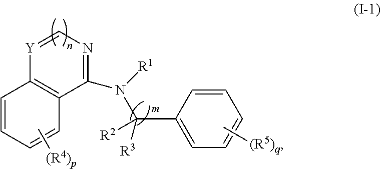
wherein:
Y is O, N, or S;
n is 0 or 1;
R⁴ and R⁵, independently for each occurrence, are halogen, cyano, nitro, alkyl, alkenyl, alkynyl, carbocyclyl, heterocyclyl, aryl, heteroaryl, —OR⁶, —NHR⁶, or —N(R⁶)₂, wherein each R⁶ is independently hydrogen, alkyl, aryl, or heteroaryl; and
p and q independently are an integer selected from 0 to 5, as valency permits.

20. The compound of any one of claims 1-19, wherein m is 0 or 1.

21. The compound of any one of claims 1-19, wherein m is 1.

22. The compound of any one of claims 1-21, wherein R² is hydrogen, alkyl, aryl or heteroaryl.

23. The compound of claim 22, wherein R² is hydrogen.

24. The compound of any one of claims 1-21, wherein R², R³, and the carbon atom to which they are connected, are taken together to complete an oxo group (C═O).

25. The compound of any one of claims 1-23, wherein R³, ring B, and the intervening atoms, complete a carbocyclyl, heterocyclyl, aryl, or heteroaryl.

26. The compound of any one of claims 1-5, wherein m is 1 and R² is hydrogen.

27. The compound of claim 1 or 2, wherein the compound of formula I is a compound of formula I-1-a: 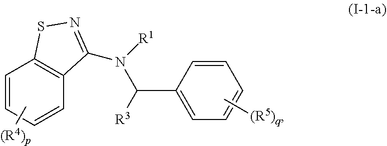
wherein:
R⁴ and R⁵, independently for each occurrence, are halogen, cyano, nitro, alkyl, alkenyl, alkynyl, carbocyclyl, heterocyclyl, aryl, or heteroaryl, —OR⁶, —NHR⁶, or —N(R⁶)₂, wherein each R⁶ is independently hydrogen, alkyl, aryl, or heteroaryl; and
p and q independently are an integer selected from 0 to 5, as valency permits.

28. The compound of claim any one of claims 1-27, wherein R³ is hydrogen, alkyl, aryl or heteroaryl.

29. The compound of claim 28, wherein R³ is hydrogen, methyl or phenyl.

30. The compound of claim 29, wherein R³ is phenyl.

31. The compound of any one of claims 17-30, wherein each R⁶ is independently aryl or alkyl optionally substituted by aryl, heteroaryl, or cycloalkyl.

32. The compound of any one of claims 17-31, wherein each R⁶ is independently methyl

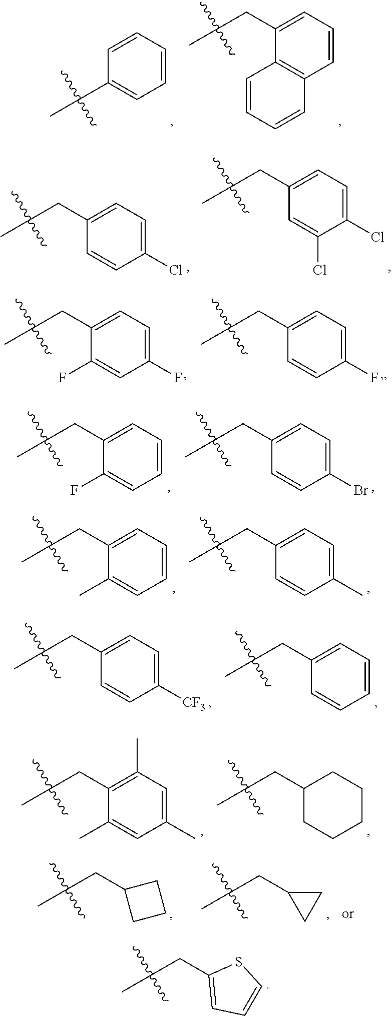

33. The compound of claim 1 or 2, wherein the compound of formula I is a compound of formula I-1-b: 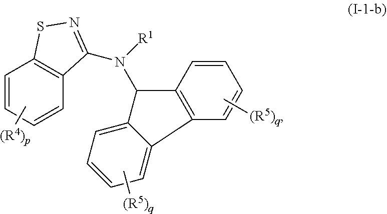
wherein:
Y is O, N, or S;
n is 0 or 1;
R⁴ and R⁵, independently for each occurrence, are halogen, amino, hydroxyl, alkoxy, cyano, nitro, alkyl, alkenyl, alkynyl, carbocyclyl, heterocyclyl, aryl, or heteroaryl; and
p and q, independently for each occurrence, are an integer selected from 0 to 5, as valency permits.

34. The compound of any one of claims 18-33, wherein each R⁴ is hydrogen, bromo, fluoro, ethynyl, cyano, benzyloxy, or methoxy.

35. The compound of claim 34, wherein R⁴ is benzyloxy optionally substituted with trifluoromethyl.

36. The compound of any one of claims 19-35, wherein each R⁵ is hydrogen, methyl, trifluoromethyl, fluoro, chloro, bromo, methoxy, trifluoromethoxy, benzyloxy, dimethylamino, —CO₂Me, cyano, nitro, difluoromethyl, or —SCF₃.

37. The compound of any one of claims 1-36, wherein R¹ is hydrogen, sulfonyl, alkyl, aralkyl, carbocyclyl, heterocyclyl, aryl, or heteroaryl.

38. The compound of claim 37, wherein R¹ is hydrogen, sulfonyl, alkyl, or aralkyl.

39. The compound of claim 38, wherein R¹ is hydrogen, methyl, ethyl, 4-trifluorobenzyl, or methylsulfonyl.

40. The compound of claim 1, wherein the compound is selected from 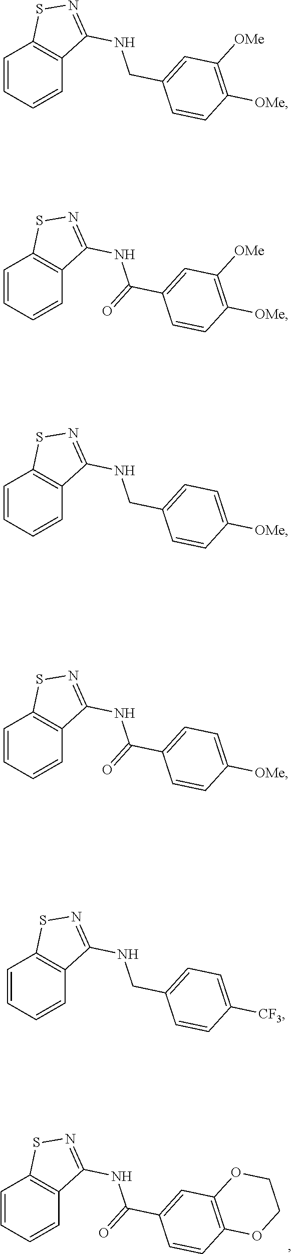 [SMILES: COc1ccc(CNc2nsc3ccccc23)cc1OC ; COc1ccc(C(=O)Nc2nsc3ccccc23)cc1OC ; COc1ccc(CNc2nsc3ccccc23)cc1 ; COc1ccc(C(=O)Nc2nsc3ccccc23)cc1 ; FC(F)(F)c1ccc(CNc2nsc3ccccc23)cc1 ; O=C(Nc1nsc2ccccc12)c1ccc2c(c1)OCCO2] 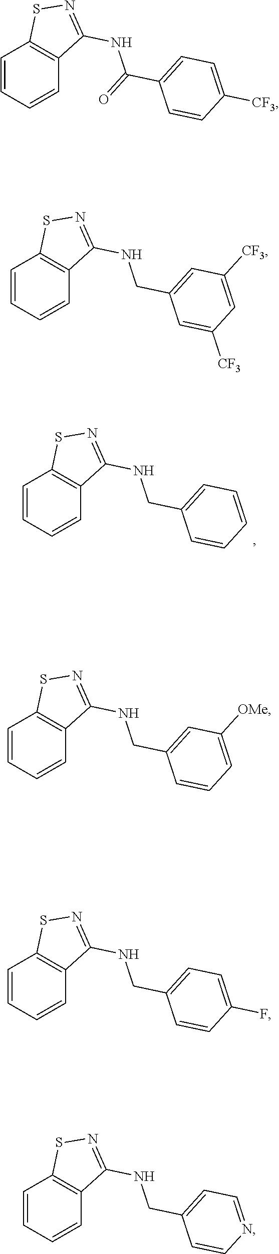 [SMILES: O=C(Nc1nsc2ccccc12)c1ccc(C(F)(F)F)cc1 ; FC(F)(F)c1cc(CNc2nsc3ccccc23)cc(C(F)(F)F)c1 ; c1ccc(CNc2nsc3ccccc23)cc1 ; COc1cccc(CNc2nsc3ccccc23)c1 ; Fc1ccc(CNc2nsc3ccccc23)cc1 ; c1ccc2c(NCc3ccncc3)nsc2c1] 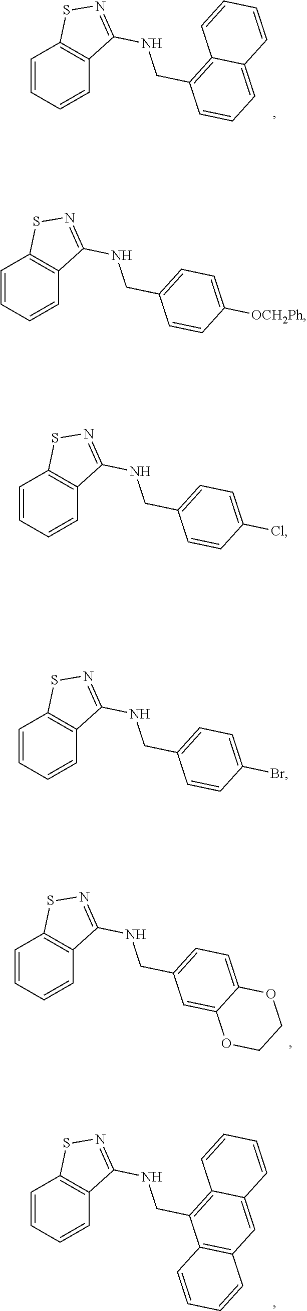 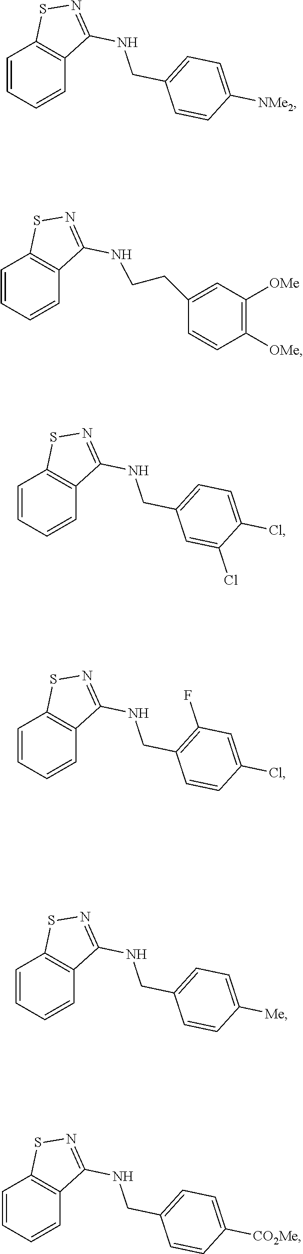 [SMILES: CN(C)c1ccc(CNc2nsc3ccccc23)cc1 ; COc1ccc(CCNc2nsc3ccccc23)cc1OC ; Clc1ccc(CNc2nsc3ccccc23)cc1Cl ; Fc1cc(Cl)ccc1CNc1nsc2ccccc12 ; Cc1ccc(CNc2nsc3ccccc23)cc1 ; COC(=O)c1ccc(CNc2nsc3ccccc23)cc1] 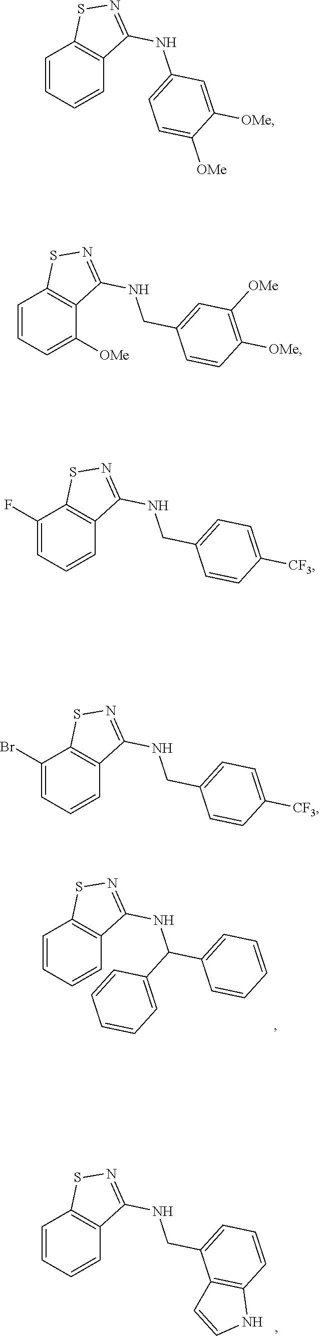 [SMILES: COc1ccc(Nc2nsc3ccccc23)cc1OC ; COc1ccc(CNc2nsc3cccc(OC)c23)cc1OC ; Fc1cccc2c(NCc3ccc(C(F)(F)F)cc3)nsc12 ; FC(F)(F)c1ccc(CNc2nsc3c(Br)cccc23)cc1 ; c1ccc(C(Nc2nsc3ccccc23)c2ccccc2)cc1 ; c1cc(CNc2nsc3ccccc23)c2cc[nH]c2c1] 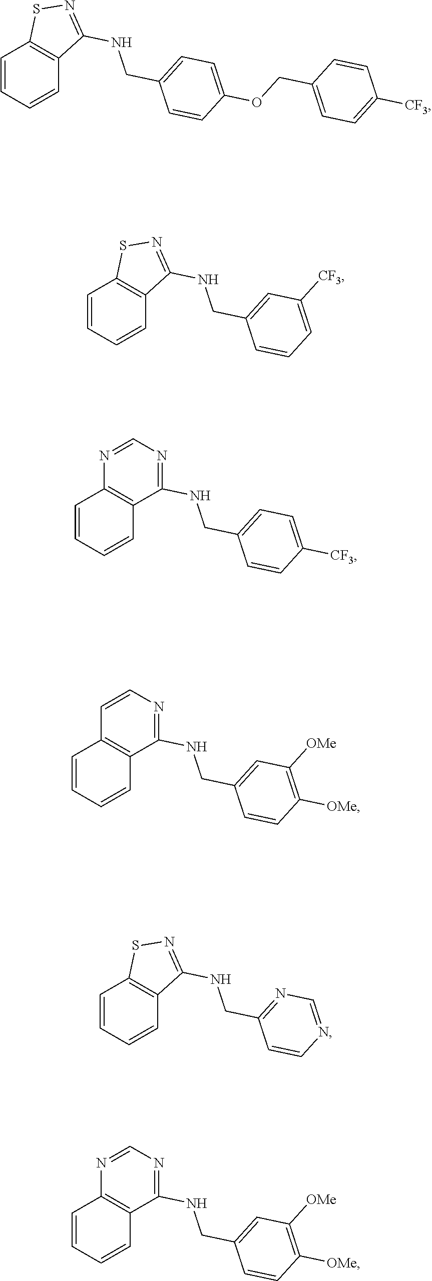 [SMILES: FC(F)(F)c1ccc(COc2ccc(CNc3nsc4ccccc34)cc2)cc1 ; FC(F)(F)c1cccc(CNc2nsc3ccccc23)c1 ; FC(F)(F)c1ccc(CNc2ncnc3ccccc23)cc1 ; COc1ccc(CNc2nccc3ccccc23)cc1OC ; c1ccc2c(NCc3ccncn3)nsc2c1 ; COc1ccc(CNc2ncnc3ccccc23)cc1OC] 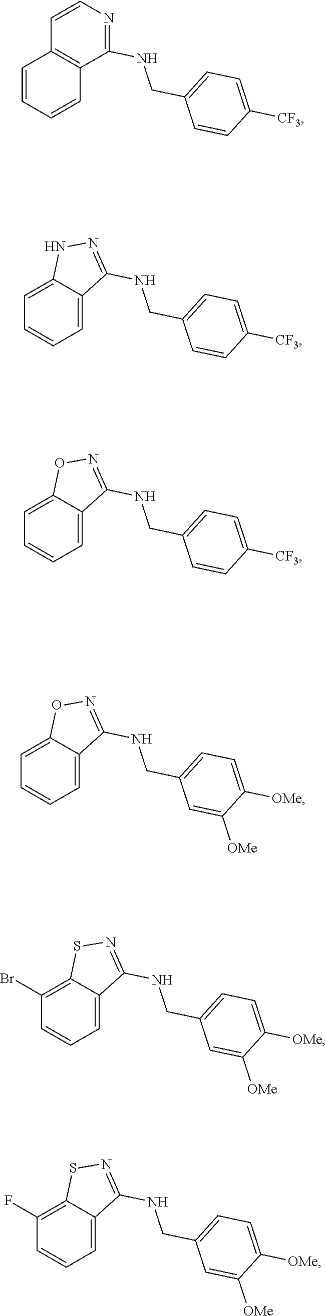 [SMILES: FC(F)(F)c1ccc(CNc2nccc3ccccc23)cc1 ; FC(F)(F)c1ccc(CNc2n[nH]c3ccccc23)cc1 ; FC(F)(F)c1ccc(CNc2noc3ccccc23)cc1 ; COc1ccc(CNc2noc3ccccc23)cc1OC ; COc1ccc(CNc2nsc3c(Br)cccc23)cc1OC ; COc1ccc(CNc2nsc3c(F)cccc23)cc1OC] 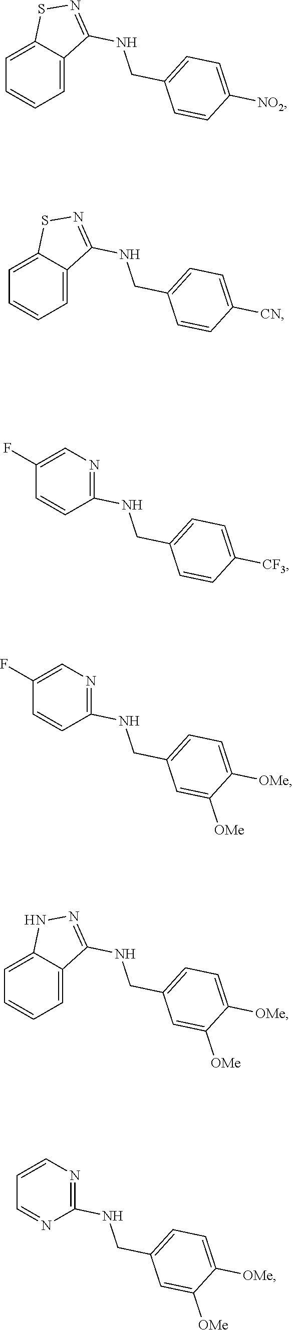 [SMILES: O=[N+]([O-])c1ccc(CNc2nsc3ccccc23)cc1 ; N#Cc1ccc(CNc2nsc3ccccc23)cc1 ; Fc1ccc(NCc2ccc(C(F)(F)F)cc2)nc1 ; COc1ccc(CNc2ccc(F)cn2)cc1OC ; COc1ccc(CNc2n[nH]c3ccccc23)cc1OC ; COc1ccc(CNc2ncccn2)cc1OC] 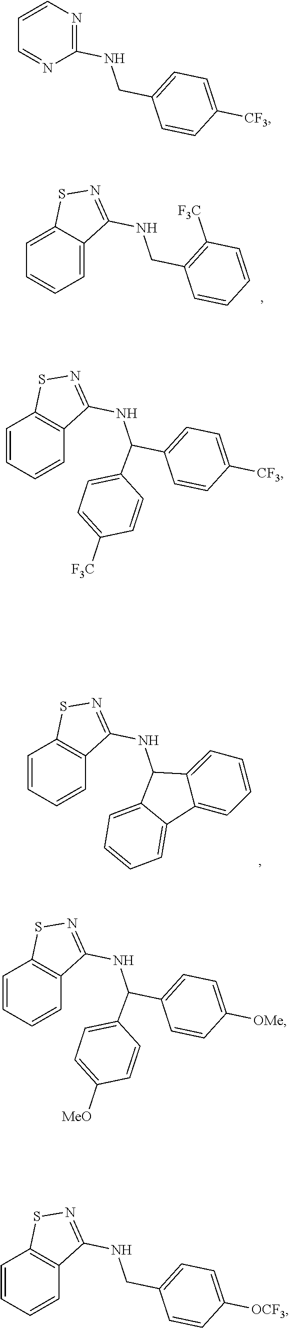 [SMILES: FC(F)(F)c1ccc(CNc2ncccn2)cc1 ; FC(F)(F)c1ccccc1CNc1nsc2ccccc12 ; FC(F)(F)c1ccc(C(Nc2nsc3ccccc23)c2ccc(C(F)(F)F)cc2)cc1 ; c1ccc2c(c1)-c1ccccc1C2Nc1nsc2ccccc12 ; COc1ccc(C(Nc2nsc3ccccc23)c2ccc(OC)cc2)cc1 ; FC(F)(F)Oc1ccc(CNc2nsc3ccccc23)cc1] 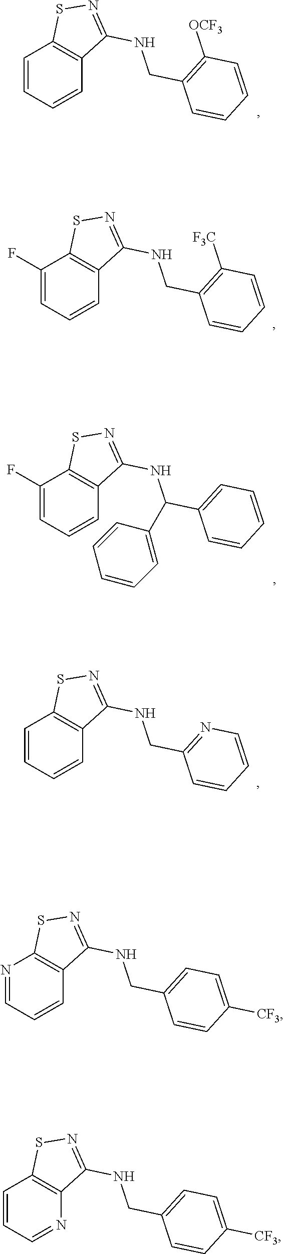 [SMILES: FC(F)(F)Oc1ccccc1CNc1nsc2ccccc12 ; Fc1cccc2c(NCc3ccccc3C(F)(F)F)nsc12 ; Fc1cccc2c(NC(c3ccccc3)c3ccccc3)nsc12 ; c1ccc(CNc2nsc3ccccc23)nc1 ; FC(F)(F)c1ccc(CNc2nsc3ncccc23)cc1 ; FC(F)(F)c1ccc(CNc2nsc3cccnc23)cc1] 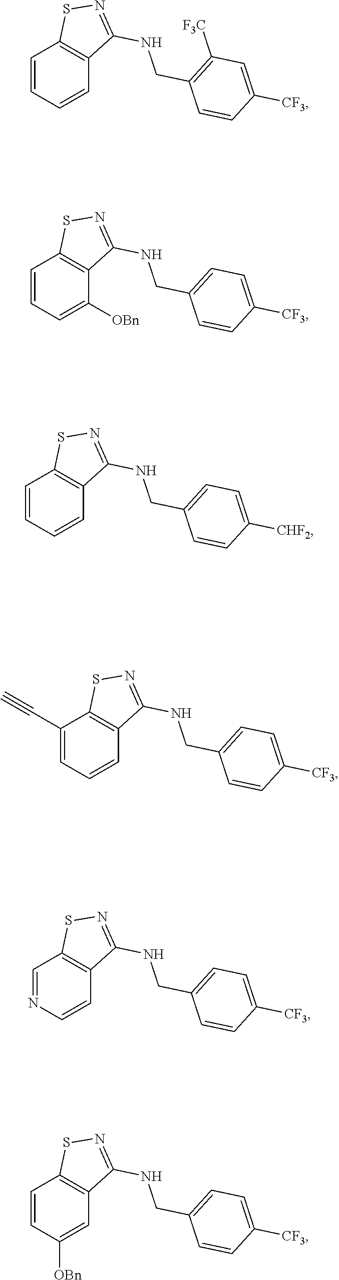 [SMILES: FC(F)(F)c1ccc(CNc2nsc3ccccc23)c(C(F)(F)F)c1 ; FC(F)(F)c1ccc(CNc2nsc3cccc(OCc4ccccc4)c23)cc1 ; FC(F)c1ccc(CNc2nsc3ccccc23)cc1 ; C#Cc1cccc2c(NCc3ccc(C(F)(F)F)cc3)nsc12 ; FC(F)(F)c1ccc(CNc2nsc3cnccc23)cc1 ; FC(F)(F)c1ccc(CNc2nsc3ccc(OCc4ccccc4)cc23)cc1] 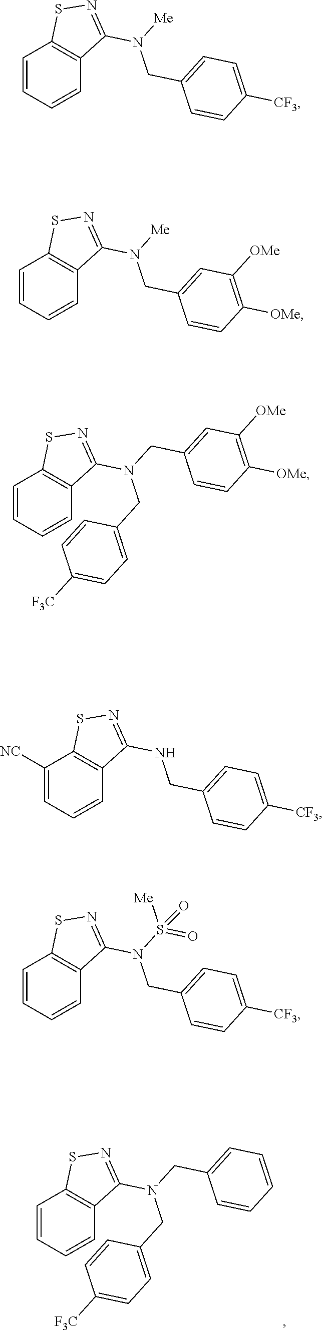 [SMILES: CN(Cc1ccc(C(F)(F)F)cc1)c1nsc2ccccc12 ; COc1ccc(CN(C)c2nsc3ccccc23)cc1OC ; COc1ccc(CN(Cc2ccc(C(F)(F)F)cc2)c2nsc3ccccc23)cc1OC ; N#Cc1cccc2c(NCc3ccc(C(F)(F)F)cc3)nsc12 ; CS(=O)(=O)N(Cc1ccc(C(F)(F)F)cc1)c1nsc2ccccc12 ; FC(F)(F)c1ccc(CN(Cc2ccccc2)c2nsc3ccccc23)cc1] 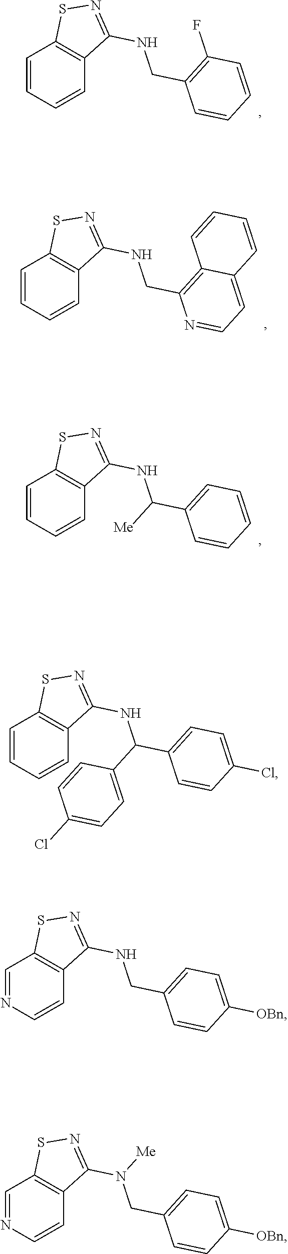 [SMILES: Fc1ccccc1CNc1nsc2ccccc12 ; c1ccc2c(CNc3nsc4ccccc34)nccc2c1 ; CC(Nc1nsc2ccccc12)c1ccccc1 ; Clc1ccc(C(Nc2nsc3ccccc23)c2ccc(Cl)cc2)cc1 ; c1ccc(COc2ccc(CNc3nsc4cnccc34)cc2)cc1 ; CN(Cc1ccc(OCc2ccccc2)cc1)c1nsc2cnccc12] 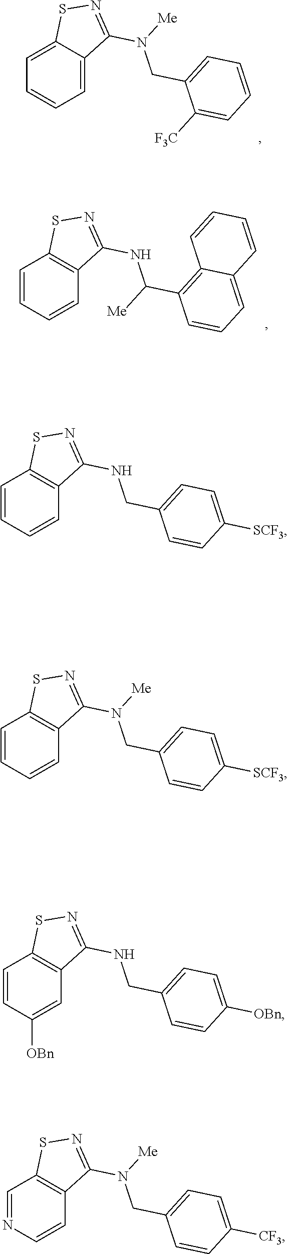 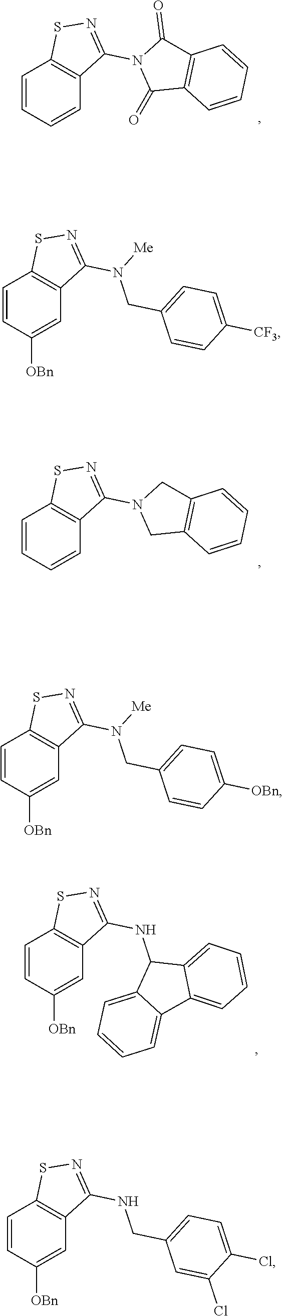 [SMILES: O=C1c2ccccc2C(=O)N1c1nsc2ccccc12 ; CN(Cc1ccc(C(F)(F)F)cc1)c1nsc2ccc(OCc3ccccc3)cc12 ; c1ccc2c(c1)CN(c1nsc3ccccc13)C2 ; CN(Cc1ccc(OCc2ccccc2)cc1)c1nsc2ccc(OCc3ccccc3)cc12 ; c1ccc(COc2ccc3snc(NC4c5ccccc5-c5ccccc54)c3c2)cc1 ; Clc1ccc(CNc2nsc3ccc(OCc4ccccc4)cc23)cc1Cl] 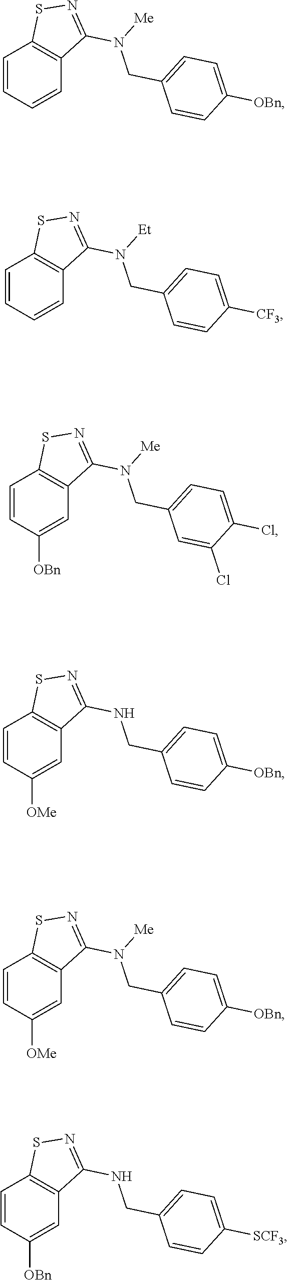 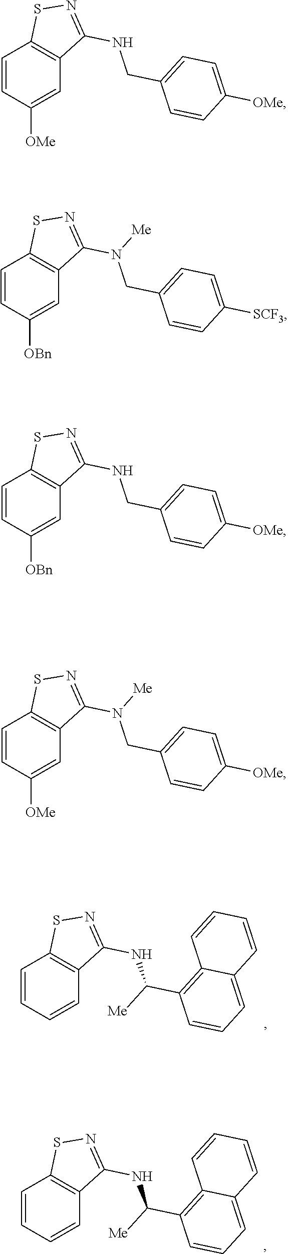 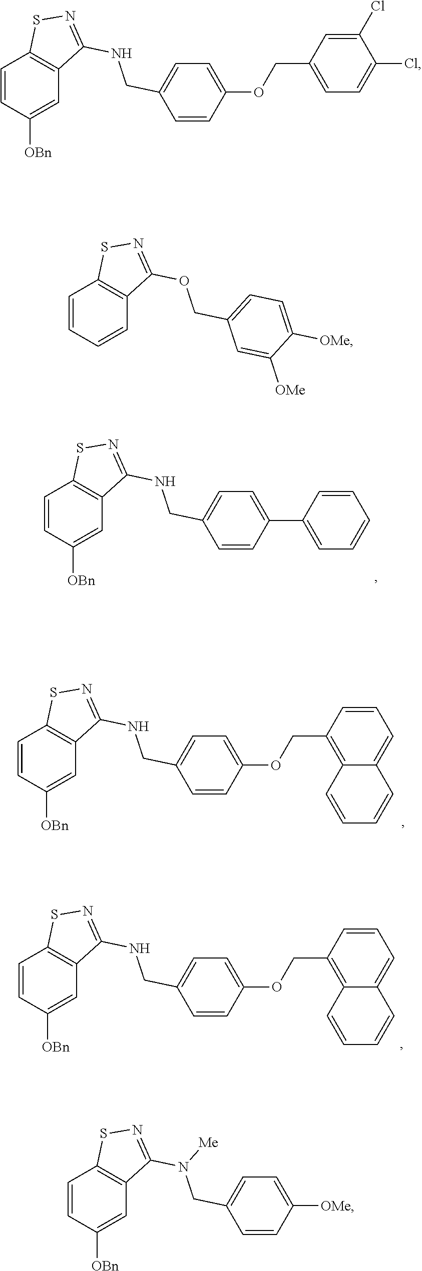 [SMILES: Clc1ccc(COc2ccc(CNc3nsc4ccc(OCc5ccccc5)cc34)cc2)cc1Cl ; COc1ccc(COc2nsc3ccccc23)cc1OC ; c1ccc(COc2ccc3snc(NCc4ccc(-c5ccccc5)cc4)c3c2)cc1 ; c1ccc(COc2ccc3snc(NCc4ccc(OCc5cccc6ccccc56)cc4)c3c2)cc1 ; c1ccc(COc2ccc3snc(NCc4ccc(OCc5cccc6ccccc56)cc4)c3c2)cc1 ; COc1ccc(CN(C)c2nsc3ccc(OCc4ccccc4)cc23)cc1] 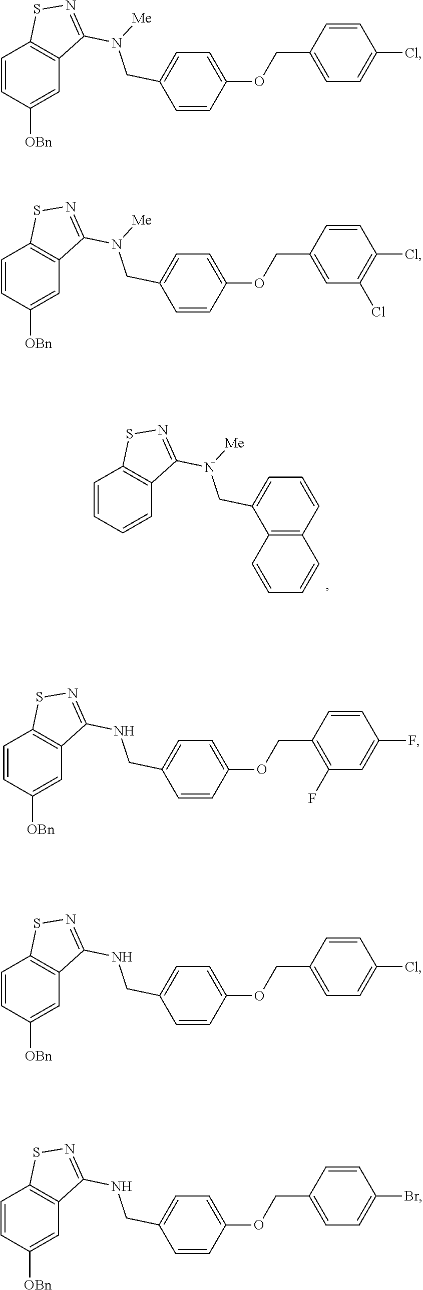 [SMILES: CN(Cc1ccc(OCc2ccc(Cl)cc2)cc1)c1nsc2ccc(OCc3ccccc3)cc12 ; CN(Cc1ccc(OCc2ccc(Cl)c(Cl)c2)cc1)c1nsc2ccc(OCc3ccccc3)cc12 ; CN(Cc1cccc2ccccc12)c1nsc2ccccc12 ; Fc1ccc(COc2ccc(CNc3nsc4ccc(OCc5ccccc5)cc34)cc2)c(F)c1 ; Clc1ccc(COc2ccc(CNc3nsc4ccc(OCc5ccccc5)cc34)cc2)cc1 ; Brc1ccc(COc2ccc(CNc3nsc4ccc(OCc5ccccc5)cc34)cc2)cc1] 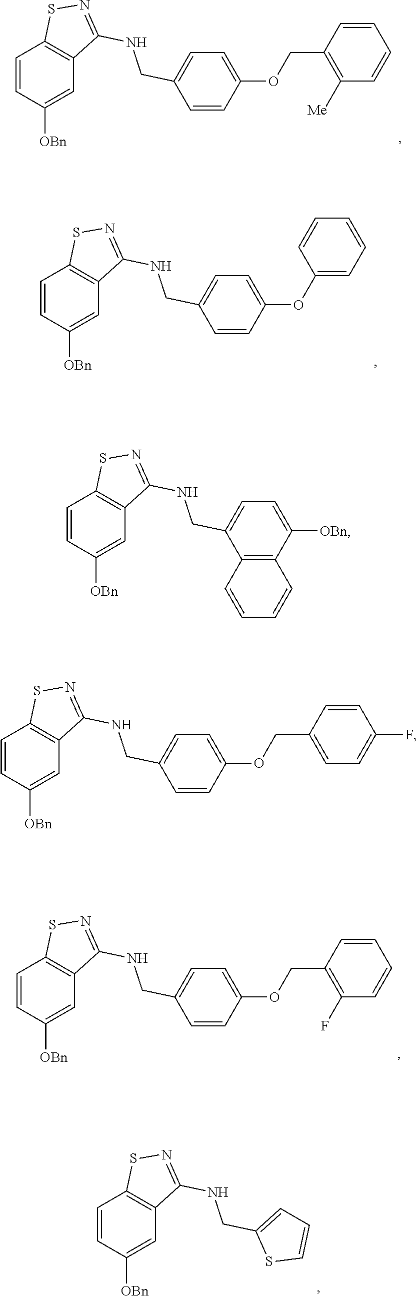 [SMILES: Cc1ccccc1COc1ccc(CNc2nsc3ccc(OCc4ccccc4)cc23)cc1 ; c1ccc(COc2ccc3snc(NCc4ccc(Oc5ccccc5)cc4)c3c2)cc1 ; c1ccc(COc2ccc3snc(NCc4ccc(OCc5ccccc5)c5ccccc45)c3c2)cc1 ; Fc1ccc(COc2ccc(CNc3nsc4ccc(OCc5ccccc5)cc34)cc2)cc1 ; Fc1ccccc1COc1ccc(CNc2nsc3ccc(OCc4ccccc4)cc23)cc1 ; c1ccc(COc2ccc3snc(NCc4cccs4)c3c2)cc1] 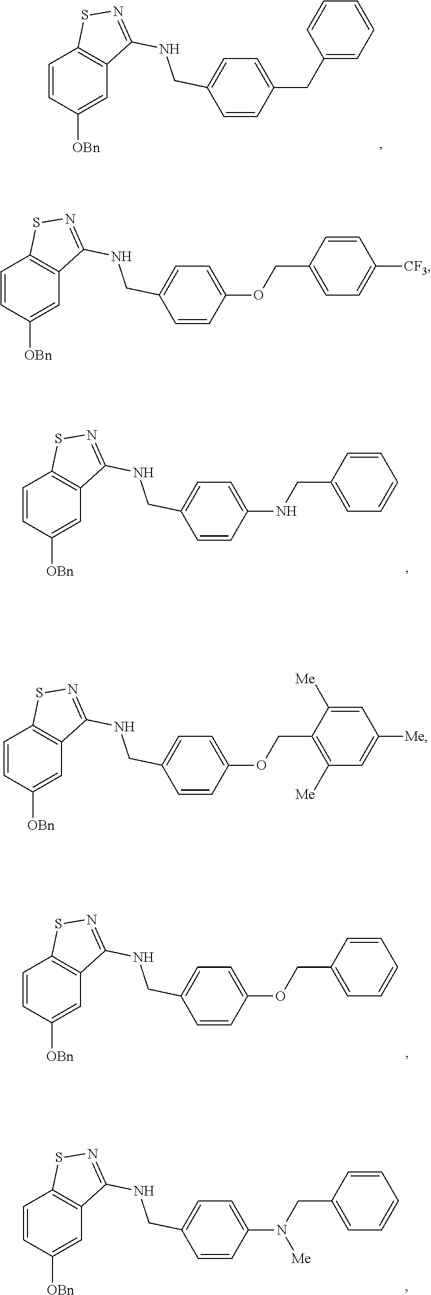 [SMILES: c1ccc(COc2ccc3snc(NCc4ccc(Cc5ccccc5)cc4)c3c2)cc1 ; FC(F)(F)c1ccc(COc2ccc(CNc3nsc4ccc(OCc5ccccc5)cc34)cc2)cc1 ; c1ccc(CNc2ccc(CNc3nsc4ccc(OCc5ccccc5)cc34)cc2)cc1 ; Cc1cc(C)c(COc2ccc(CNc3nsc4ccc(OCc5ccccc5)cc34)cc2)c(C)c1 ; c1ccc(COc2ccc(CNc3nsc4ccc(OCc5ccccc5)cc34)cc2)cc1 ; CN(Cc1ccccc1)c1ccc(CNc2nsc3ccc(OCc4ccccc4)cc23)cc1] 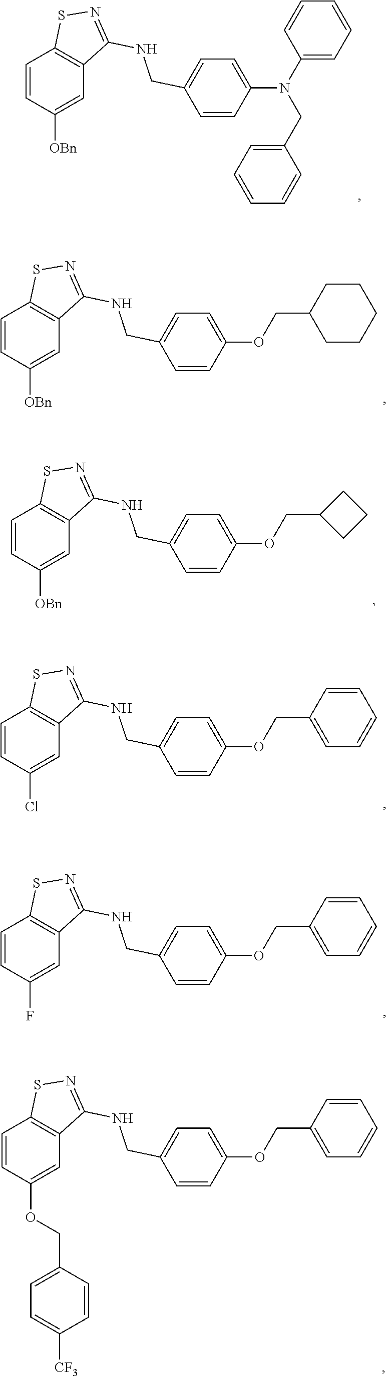 [SMILES: c1ccc(COc2ccc3snc(NCc4ccc(N(Cc5ccccc5)c5ccccc5)cc4)c3c2)cc1 ; c1ccc(COc2ccc3snc(NCc4ccc(OCC5CCCCC5)cc4)c3c2)cc1 ; c1ccc(COc2ccc3snc(NCc4ccc(OCC5CCC5)cc4)c3c2)cc1 ; Clc1ccc2snc(NCc3ccc(OCc4ccccc4)cc3)c2c1 ; Fc1ccc2snc(NCc3ccc(OCc4ccccc4)cc3)c2c1 ; FC(F)(F)c1ccc(COc2ccc3snc(NCc4ccc(OCc5ccccc5)cc4)c3c2)cc1] 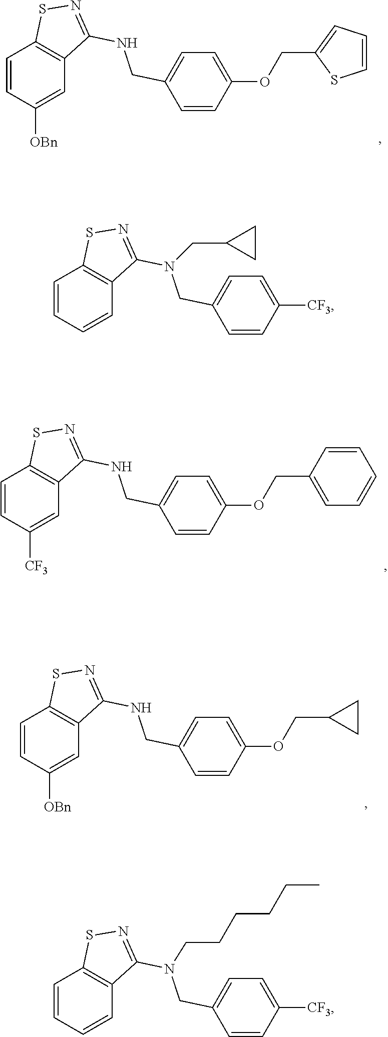 [SMILES: c1ccc(COc2ccc3snc(NCc4ccc(OCc5cccs5)cc4)c3c2)cc1 ; FC(F)(F)c1ccc(CN(CC2CC2)c2nsc3ccccc23)cc1 ; FC(F)(F)c1ccc2snc(NCc3ccc(OCc4ccccc4)cc3)c2c1 ; c1ccc(COc2ccc3snc(NCc4ccc(OCC5CC5)cc4)c3c2)cc1 ; CCCCCCN(Cc1ccc(C(F)(F)F)cc1)c1nsc2ccccc12] 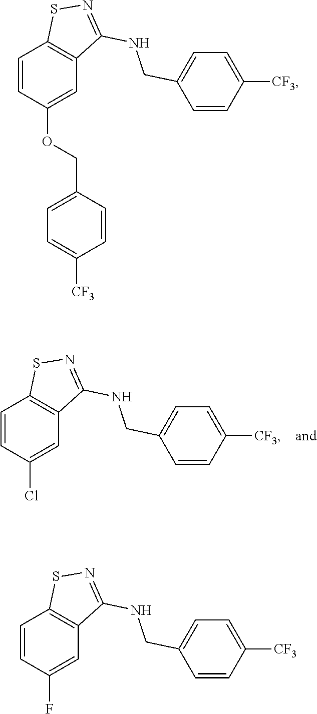 [SMILES: FC(F)(F)c1ccc(CNc2nsc3ccc(OCc4ccc(C(F)(F)F)cc4)cc23)cc1 ; FC(F)(F)c1ccc(CNc2nsc3ccc(Cl)cc23)cc1 ; Fc1ccc2snc(NCc3ccc(C(F)(F)F)cc3)c2c1]
or a pharmaceutically acceptable salt thereof.

41. A pharmaceutical composition, comprising a compound of any one of claims 1-40 and a pharmaceutically acceptable excipient.

42. A method of treating a proteopathy, comprising administering to a subject in need thereof a compound of any one of claims 1 to 40 or a composition of claim 41.

43. The method of claim 42, wherein the proteopathy is a tau-associated neurodegenerative disease selected from Alzheimer's disease, Progressive supranuclear palsy, Corticobasal degeneration, Frontotemporal dementia, Frontotemporal dementia and parkinsonism linked to chromosome 17, Pick's disease, Argyrophilic grain disease, Globular glial tauopathies, Aging-related tau astrogliopathy, Chronic traumatic encephalopathy, Primary age-related tauopathy, Parkinsonism-dementia complex of Guam, Postencephalitic parkinsonism, Atypical Parkinsonism of Guadeloupe, Diffuse neurofilament tangles with calcification, Subacute sclerosing panencephalitis, Lytico-bodig disease, Pantothenate kinase-associated neurodegeneration, and Lipofuscinosis.

44. The method of claim 42, wherein the proteopathy is a neurodegenerative disease selected from Alzheimer's disease (AD) and AD-related disorders, Parkinson's disease (PD) and PD-related disorders, Huntington's disease and other trinucleotide repeat disorders, Spinocerebellar ataxia (SCA, including SCA 2, SCA 3, SCA 6, SCA 7, SCA 17), Amyotrophic lateral sclerosis, prion disease, Frontotemporal lobar degeneration, Hallervorden-Spatz disease, neuroaxonal dystrophies, familial encephalopathy accompanied by neuroserpin inclusion bodies, Multiple System Atrophy, and Dentatorubralpallidoluysian Atrophy.

45. The method of claim 42, wherein the proteopathy is a dementia selected from Alzheimer's disease (AD) and AD-related disorders, Familial Alzheimer's disease, Dementia with Lewy Bodies (dementia accompanied by Lewy bodies), Dementia in Parkinson's disease, Frontotemporal Degeneration, Frontotemporal Dementia, Frontotemporal Dementia with parkinsonism linked to chromosome 17, Primary Progressive Aphasia, Semantic Dementia, Pick's disease, Dementia lacking distinctive histology, Familial British dementia, Familial Danish dementia, dementia pugilistica, and tangle-predominant dementia.

46. The method of claim 42, wherein the proteopathy is an amyloidosis or a disease that is caused by or associated with protein aggregation or protein pathology selected from Aβ amyloidosis, AL (light chain) amyloidosis (primary systemic amyloidosis), AH (heavy chain) amyloidosis, AA (secondary) amyloidosis, Aortic medial amyloidosis, apolipoprotein AI amyloidosis (AApoAI), apolipoprotein All amyloidosis (AApoAII), apolipoprotein AIV amyloidosis (AApoAIV), Familial amyloidosis of the Finnish type, Lysozyme amyloidosis, Fibrinogen amyloidosis, Dialysis amyloidosis, Cardiac atrial amyloidosis, Cutaneous lichen amyloidosis, primary cutaneous amyloidosis, Corneal lactoferrin amyloidosis, Lect2 amyloidosis, islet amyloid polypeptide amyloidosis, Hereditary cerebral hemorrhage with amyloidosis, Familial amyloidotic neuropathy, Senile systemic amyloidosis, Mallory bodies, Medullary thyroid carcinoma, Pituitary prolactinoma, Hereditary lattice corneal dystrophy, Odontogenic (Pindborg) tumor amyloid, Seminal vesicle amyloid, Apolipoprotein C2 amyloidosis, Apolipoprotein C3 amyloidosis, Insulin amyloidosis, Galectin-7 amyloidosis (primary localized cutaneous amyloidosis), Corneodesmosin amyloidosis, Enfuvirtide amyloidosis, Cerebral β-amyloid angiopathy, Retinal ganglion cell degeneration in glaucoma, Alexander disease, Pelizaeus-Merzbacher disease, Seipinopathies, Serpinopathies, Inclusion body myositis/myopathy, Cataracts, Retinitis pigmentosa with rhodopsin mutations, Pulmonary alveolar proteinosis, Type II diabetes, Cystic fibrosis, Sickle cell disease, neuronal intranuclear hyaline inclusion disease, and transthyretine-associated cerebral amyloidosis.

47. The method of claim 42, wherein the proteopathy is a TDP-43 proteinopathy selected from amyotrophic lateral sclerosis, frontotemporal lobar degeneration, limbic-predominant age-related TDP-43 encephalopathy, and Perry syndrome.

48. The method of claim 42, wherein the proteopathy is a synucleinopathy selected from diseases with Lewy bodies, Parkinson disease, Parkinson-plus syndrome, multiple systemic atrophy, Shy-Drager syndrome, MSA-P (striatonigral degeneration), and olivopontocerebellar atrophy.

49. The method of claim 42, wherein the proteopathy is Alzheimer's disease.

50. The method of claim 42, wherein the proteopathy is Parkinson's Disease.

51. The method of any one of claims 42-45, wherein the compound is administered intravenously.

52. The method of any one of claims 42-45, wherein the compound is administered orally.
